(19) United States
(12) Patent Application Publication
(10) Pub. No.: US 2022/0267298 A1
(43) Pub. Date: Aug. 25, 2022

(54) QUINAZOLIN-4-ONE DERIVATIVES USEFUL AS GRK2 INHIBITORS

(71) Applicant: Janssen Pharmaceutica NV, Beerse (BE)

(72) Inventors: Guozhang Xu, Chesterbrook, PA (US); Micheal D. Gaul, Apex, NC (US)

(21) Appl. No.: 17/630,537

(22) PCT Filed: Jul. 29, 2020
(86) PCT No.: PCT/EP2020/071370

§ 371 Date: Jan. 27, 2022

Related U.S. Application Data
(60) Provisional application No. 62/880,305, filed on Jul. 30, 2019.

(51) Int. Cl. C07D 401/10; C07D 403/10; C07D 405/14; C07D 401/14; C07D 413/14; C07D 409/14; C07D 471/04

(52) U.S. Cl. CPC C07D 401/10; C07D 403/10; C07D 405/14; C07D 471/04; C07D 413/14; C07D 409/14; C07D 401/14

(57) ABSTRACT

The present invention is directed to quinazolin-4-one derivatives, pharmaceutical compositions containing them and their use in the treatment of disorders and conditions modulated by GRK2, including the treatment of for example, cardiac failure, cardiac hypertrophy, hypertension, Type II diabetes Mellitus, NASH, NAFLD, End-stage kidney disease, kidney failure, etc.

### CROSS REFERENCE TO RELATED APPLICATIONS

This application claims the benefit of U.S. Provisional Application 62/880,305, filed on Jul. 30, 2019, which is incorporated by reference herein in its entirety.

### FIELD OF THE INVENTION

The present invention is directed to quinazolin-4-one derivatives, pharmaceutical compositions containing them and their use in the treatment of disorders and conditions modulated by GRK2. More particularly, the compounds of the present invention are useful in the treatment of for example, cardiac failure, cardiac hypertrophy, hypertension, Type II diabetes Mellitus, NASH, NAFLD, End-stage kidney disease, kidney failure, etc.

### BACKGROUND OF THE INVENTION

G-protein-coupled receptor kinase 2 (GRK2) is a G-protein-coupled receptor kinase that is ubiquitously expressed in many tissues and regulates various intracellular mechanisms. The up- or down-regulation of GRK2 correlates with several pathological disorders. GRK2 plays an important role in the maintenance of heart structure and function; thus, this kinase is involved in many cardiovascular diseases. GRK2 up-regulation can worsen cardiac ischemia; furthermore, increased kinase levels occur during the early stages of heart failure and in hypertensive subjects. GRK2 up-regulation can lead to changes in the insulin signaling cascade, which can translate to insulin resistance. Increased GRK2 levels also correlate with the degree of cognitive impairment that is typically observed in Alzheimer's disease. (GUCCIONE, M., et al., “G-Protein-Coupled Receptor Kinase 2 (GRK2) Inhibitors: Current Trends and Future Perspectives”, J. Med. Chem, 2016, pp 9277-9294, Vol 59 (20)).

GRK2 is a prototypic GRK. This cytosolic protein is ubiquitously expressed in many tissues, but it is particularly important for embryonic development and heart function. GRK2 plays a key role in several signal transduction pathways. This protein can trigger receptor desensitization and internalization through R-arrestin binding to activated GPCRs. GRK2 can also phosphorylate different effectors involved in signal transduction. Moreover, the expression and/or function of GRK2 is altered in several pathological conditions, including cardiovascular and inflammatory pathologies.

Heart failure (HF) is the most common disease for hospitalization in the elderly, with approximately 10% of men and 8% of women over the age of 60 affected. The prevalence of HF is growing with the rise of an aging population in developed countries. There remains an intense need for novel beneficial HF therapies, with more than 3 million people in the United States diagnosed per year, and HF related mortality and rehospitalization rates remaining high despite the modest improvement in survival rates seen from advances in device therapy and pharmacological therapy (angiotensin II receptor blockers, angiotensin converting enzyme inhibitors, and β-blockers). A plethora of research into HF has revealed it to be a complex disease associated with various pathogenetic mechanisms, including ventricular remodeling, excessive neurohormonal stimulation, abnormal Ca²⁺ handling, and proliferation of the extracellular matrix. Although an overstimulation of the sympathetic nervous system (SNS) initially compensates for cardiac dysfunction, the subsequent release of catecholamine ultimately promotes disease progression via long-term exposure. Activation of the SNS is mediated by adrenergic receptors (AR), and chronic β-AR activation induces β-AR desensitization and downregulation, subsequently leading to the reduction of β-AR signaling. G-protein receptor kinase (GRK) 2 phosphorylates agonist-occupied β-AR, promotes the binding of β-AR arrestin to the Gβγ subunit of the G-protein, facilitates the G-protein uncoupling from β-AR, and results in β-AR desensitization and downregulation. In the hearts of HF patients, GRK2 expression levels and activity were elevated, accompanied by lowered β-AR density and signaling. Moreover, GRK2 inhibition by overexpression of the βARKct, the peptide inhibitor of GRK2, or cardiac specific GRK2 gene ablation, improved cardiac function and survival with the increases in β-AR density and β-AR responses in several HF models. These results suggest that GRK2 has a strong relationship with HF, and inhibition of GRK2 is a promising mechanism for the treatment of HF (OKAWA, T., et al., J. Med. Chem., 2017, pp 6942-6990, Vol. 60).

G protein-coupled receptor kinase 2 (GRK2) is emerging as a pivotal signalling hub able to integrate different transduction cascades. This ability appears to underlie its central role in different physiological and pathological conditions. Key mediators of cardiovascular function (such as catecholamines or angiotensin II) and components of the systemic milieu altered in insulin resistance conditions converge in increasing GRK2 levels in diverse cardiovascular cell types. In turn, GRK2 would simultaneously modulate several cardiovascular regulatory pathways, including GPCR and insulin signalling cascades, NO bioavailability and mitochondrial function. This fact can help explain the contribution of increased GRK2 levels to maladaptive cardiovascular function and remodeling. It also unveils GRK2 as a link between cardiovascular pathologies and co-morbidities such as obesity or type 2 diabetes. On the other hand, enhanced GRK2 expression, as observed in adipose tissues, liver or skeletal muscle during insulin resistance-related pathologies, could modify the orchestration of GPCR and insulin signalling in these crucial metabolic organs, and contribute to key features of the obese and insulin-resistant phenotype (MAYOR, Jr., F., et al., Cellular Signalling, 2018, pp 25-32, Vol. 41).

FENG, Y, et al., in PCT Publication WO2010/056758, Published 20 May 2010 describe quinazoline compounds that can inhibit the bioactivity of one or more kinase enzymes. BARLAAM, B. et al., in Tetrahedron, 2012, pp 534-543, Vol 68(2) describe the preparation of 6-aminoquinazolin-4(3H)-ones.

There remains a need for GRK2 inhibitor compounds that have pharmacokinetic and pharmacodynamic properties suitable for use as human pharmaceuticals for the treatment of for example, cardiac failure, cardiac hypertrophy, hypertension, Type II diabetes Mellitus, NASH, NAFLD, chronic kidney disease, end-stage kidney disease, kidney failure, etc.

### SUMMARY OF THE INVENTION

The present invention is directed to compounds of formula (I)

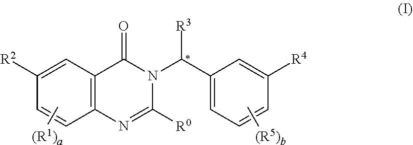

wherein

R⁰ is selected from the group consisting of hydrogen, C₁₋₄alkyl, fluorinated C₁₋₂alkyl, C₁₋₄alkoxy, fluorinated C₁₋₂alkoxy and 4 to 7 membered, nitrogen containing, saturated heterocyclyl; (wherein the 4 to 7 membered, nitrogen containing, saturated heterocyclyl contains at least one nitrogen atom;)

wherein the 4 to 7 membered, nitrogen containing, saturated heterocyclyl is optionally substituted with one or more substituents independently selected from the group consisting of hydroxy and NR^{X}R^{Y}; wherein R^{X} and R^{Y} are each independently selected from the group consisting of hydrogen and C₁₋₄alkyl;

a is an integer from 0 to 3;

each R¹ is independently selected from the group consisting of halogen, hydroxy, C₁₋₄alkyl, fluorinated C₁₋₂alkyl, C₁₋₄alkoxy, fluorinated C₁₋₂alkoxy and cyano;

R² is selected from the group consisting of 5 to 6 membered heteroaryl and 1H-pyrrolo[2,3-b]pyridin-3-yl; wherein the 5 to 6 membered heteroaryl is optionally substituted with one or more substituents independently selected from the group consisting of halogen, C₁₋₄alkyl, fluorinated C₁₋₂alkyl, oxo and NR^{A}R^{B}; wherein R^{A} and R^{B} are each independently selected from the group consisting of hydrogen and C₁₋₂alkyl;

R³ is selected from the group consisting of hydrogen, —C₁₋₄alkyl, —C₁₋₄alkoxy, —(C₁₋₂alkyl)-OH, —(C₁₋₂alkyl)-NR^{C}R^{D}, —(C₁₋₂alkyl)-SO₂—(C₁₋₂alkyl), —CO₂H, —C(O)O—(C₁₋₂alkyl) and tetrahydropyran-4-yl-1,1-dioxide; wherein R^{C} and R^{D} are each independently selected from the group consisting of hydrogen and C₁₋₂alkyl;

R⁴ is selected from the group consisting of hydrogen, halogen, hydroxy, C₁₋₄alkyl, fluorinated C₁₋₂alkyl, C₁₋₄alkoxy, fluorinated C₁₋₂alkoxy, —(C₁₋₂alkyl)-CO₂H, —(C₁₋₂alkyl)-C(O)O—(C₁₋₄alkyl), —O—C₂₋₄alkynyl, —O—(C₁₋₂alkyl)-C(O)OH, —O—(C₁₋₂alkyl)-C(O)O—(C₁₋₂alkyl), —O—(C₁₋₂alkyl)-O—(C₃₋₅cycloalkyl), —O—(C₁₋₂alkyl)-C(O)-morpholine, —O—(C₁₋₂alkyl)-C(O)—NR^{E}R^{F}, —O—(C₁₋₂alkyl)-C(O)—NH—(C₃₋₅cycloalkyl), —O—(C₁₋₂alkyl)-SO₂—(C₁₋₂alkyl), —O—(C₃₋₆cycloalkyl), —O-phenyl, —O-benzyl, —O-azetidin-3-yl, —O-(1-methyl-azetidin-3-yl), —O-pyrrolidin-3-yl, —O-(1-methyl-pyrrolidin-3-yl), —O-piperidin-4-yl, —O-(1-methyl-piperidin-4-yl), —C(O)—(C₁₋₂alkyl), —C(O)—NR^{E}R^{F}, —C(O)—NH—(C₂₋₄alkynyl), —C(O)—NH—(C₂alkyl)-CO₂H, —C(O)—NH—(C₂alkyl)-C(O)O—(C₁₋₂alkyl), —C(O)—NH-(phenyl), —C(O)—NH-(benzyl), —C(O)—NH—(C₃₋₈cycloalkyl), —C(O)—NH-(pyridinyl), —C(O)—NH—(CH₂CH₂-morpholin-4-yl), —C(O)—NH-(azetidin-3-yl), —C(O)—NH-(1-methyl-azetidin-3-yl), —C(O)—NH-pyrrolidin-3-yl, —C(O)—NH-(1-methyl-pyrrolidin-3-yl), —C(O)—NH-piperidin-4-yl, —C(O)—NH-(1-methyl-piperidin-4-yl), —NH—SO₂—(C₁₋₂alkyl), —S—(C₁₋₄alkyl), —SO—(C₁₋₄alkyl), —SO₂—(C₁₋₄alkyl), —SO₂—NR^{E}R^{F}, and oxazol-2-yl;

wherein the phenyl or benzyl, whether alone or as part of a substituent group, is optionally substituted with one to two substituents independently selected from the group consisting of halogen, C₁₋₄alkyl and C₁₋₄alkoxy;

and wherein R^{E} and R^{F} are each independently selected form the group consisting of hydrogen and C₁₋₄alkyl;

b is an integer from 0 to 4;

each R⁵ is independently selected from the group consisting of halogen, C₁₋₄alkyl and C₁₋₄alkoxy;

provided than when R⁰ is hydrogen or methyl, a is an integer from 0 to 1, R¹, when present, is 8-methyl, R³ is hydrogen, R⁴ is methoxy, and b is 0, then R² is other than pyrazol-4-yl or imidazol-1-yl;

and stereoisomers, tautomers, isotopologues, isotopomers, and pharmaceutically acceptable salts thereof.

The present invention is further directed to a compound selected from the group consisting of a compound of formula (C1)

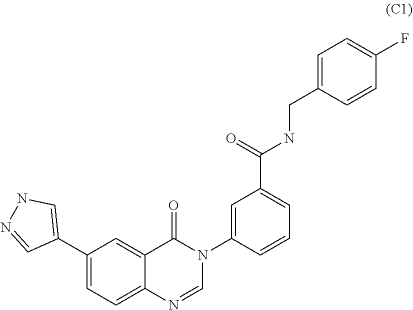 [SMILES: O=C(NCc1ccc(F)cc1)c1cccc(-n2cnc3ccc(-c4cn[nH]c4)cc3c2=O)c1]

also known as N-(4-fluorobenzyl)-3-(4-oxo-6-(1H-pyrazol-4-yl)quinazolin-3(4H)-yl)benzamide;

a compound of formula (C2)

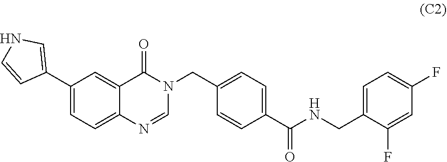 [SMILES: O=C(NCc1ccc(F)cc1F)c1ccc(Cn2cnc3ccc(-c4cc[nH]c4)cc3c2=O)cc1]

also known as N-(2,4-difluorobenzyl)-4-((4-oxo-6-(1H-pyrrol-3-yl)quinazolin-3(4H)-yl)methyl)benzamide;

a compound of formula (C3)

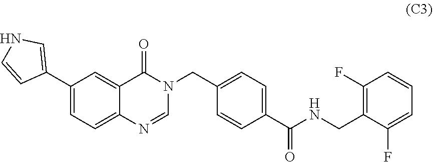 [SMILES: O=C(NCc1c(F)cccc1F)c1ccc(Cn2cnc3ccc(-c4cc[nH]c4)cc3c2=O)cc1]

also known as N-(2,6-difluorobenzyl)-4-((4-oxo-6-(1H-pyrrol-3-yl)quinazolin-3(4H)-yl)methyl)benzamide;

a compound of formula (C4)

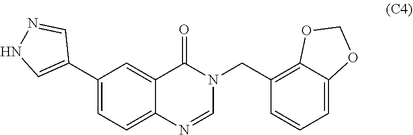 [SMILES: O=c1c2cc(-c3cn[nH]c3)ccc2ncn1Cc1cccc2c1OCO2]

also known as 3-(benzo[d][1,3]dioxol-4-ylmethyl)-6-(1H-pyrazol-4-yl)quinazolin-4(3H)-one;

a compound of formula (C5)

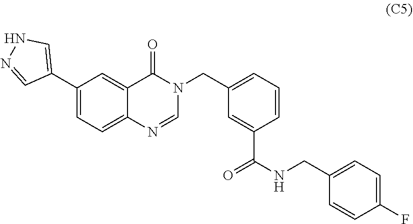 [SMILES: O=C(NCc1ccc(F)cc1)c1cccc(Cn2cnc3ccc(-c4cn[nH]c4)cc3c2=O)c1]

also known as N-(4-fluorobenzyl)-4-((4-oxo-6-(1H-pyrazol-4-yl)quinazolin-3(4H)-yl)methyl)picolinamide; and

a compound of formula C6

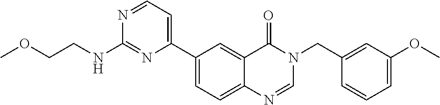

also known as 3-(3-methoxybenzyl)-6-(2-((2-methoxyethyl)amino)pyrimidin-4-yl)quinazolin-4(3H)-one;

and stereoisomers, tautomers, isotopologues, isotopomers, and pharmaceutically acceptable salts thereof.

The present invention is further directed to a compound of formula (D)

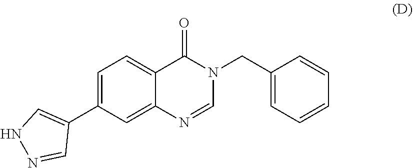 [SMILES: O=c1c2ccc(-c3cn[nH]c3)cc2ncn1Cc1ccccc1]

also known as 3-benzyl-7-(1H-pyrazol-4-yl)quinazolin-4(3H)-one, and stereoisomers, tautomers, isotopologues, isotopomers, and pharmaceutically acceptable salts thereof. The compound of formula (D) is a GRK2 inhibitor, with an IC₅₀ of 4.1 μM (a measured by enzymatic activity assay described in Biological Example 2, hereinafter).

The present invention is further directed to processes for the preparation of the compounds of formula (I). The present invention is further directed to a compound of formula (I) prepared according to any of the process(es) described herein.

The present invention is further directed to processes for the preparation of the compounds of formula (C1), (C2), (C3), (C4), (C5) and (C6). The present invention is further directed to a compound of formula (C1), (C2), (C3), (C4), (C5) or (C6) prepared according to any of the process(es) described herein.

The present invention is further directed to processes for the preparation of the compounds of formula (D). The present invention is further directed to a compound of formula (D) prepared according to any of the process(es) described herein.

Illustrative of the invention are pharmaceutical compositions comprising a pharmaceutically acceptable carrier and a compound of formula (I) as described herein. An illustration of the invention is a pharmaceutical composition made by mixing a compound of formula (I) as described herein and a pharmaceutically acceptable carrier. Illustrating the invention is a process for making a pharmaceutical composition comprising mixing a compound of formula (I) as described herein and a pharmaceutically acceptable carrier.

Illustrative of the invention are pharmaceutical compositions comprising a pharmaceutically acceptable carrier and a compound of formula (C1), (C2), (C3), (C4), (C5) or (C6) as described herein. An illustration of the invention is a pharmaceutical composition made by mixing a compound of formula (C1), (C2), (C3), (C4), (C5) or (C6) as described herein and a pharmaceutically acceptable carrier. Illustrating the invention is a process for making a pharmaceutical composition comprising mixing a compound of formula (C1), (C2), (C3), (C4), (C5) or (C6) as described herein and a pharmaceutically acceptable carrier.

Exemplifying the invention are methods of treating a disease, disorder, or condition mediated by GRK2 activity as described herein, comprising administering to a subject in need thereof a therapeutically effective amount of any of the compounds or pharmaceutical compositions described above.

Exemplifying the invention are methods of treating a disease, disorder, or condition mediated by GRK2 activity such as obesity, excess weight, impaired glucose tolerance (IGT), impaired fasting glucose (IFT), gestational diabetes, Type II diabetes mellitus, Syndrome X (also known as Metabolic Syndrome), nephropathy, neuropathy, retinopathy, cardiac failure, cardiac hypertrophy, cardiac fibrosis, hypertension, angina, atherosclerosis, heart disease, heart attack, ischemia, stroke, nerve damage or poor blood flow in the feet, sepsis-associated encephalopathy (SAE), non-alcoholic steatohepatitis (NASH), non-alcoholic fatty liver disease (NAFLD) and renal disorders (including, but not limited to end-stage kidney disease, chronic kidney disease, acute renal failure, nephrotic syndrome, renal hyperfiltrative injury, hyperfiltrative diabetic nephropathy, renal hyperfiltration, glomerular hyperfiltration, renal allograft hyperfiltration, compensatory hyperfiltration, hyperfiltrative chronic kidney disease, hyperfiltrative acute renal failure, a measured GFR equal or greater than 125 mL/min/1.73 m² (for example, a measured GFR equal or greater than 140 mL/min/1.73 m²)), comprising administering to a subject in need thereof a therapeutically effective amount of any of the compounds or pharmaceutical compositions described above.

In an embodiment, the present invention is directed to a compound as described herein (e.g. a compound of formula (I), formula C1, formula C2, formula C3, formula C4, formula C5, formula C6 or formula (D)) for use as a medicament. In another embodiment, the present invention is directed to a compound as described herein (e.g. a compound of formula (I), formula C1, formula C2, formula C3, formula C4, formula C5, formula C6 or formula (D)) for use in the treatment of a disorder mediated GRK2 activity such as obesity, excess weight, impaired glucose tolerance (IGT), impaired fasting glucose (IFT), gestational diabetes, Type II diabetes mellitus, Syndrome X (also known as Metabolic Syndrome), nephropathy, neuropathy, retinopathy, cardiac failure, cardiac hypertrophy, cardiac fibrosis, hypertension, angina, atherosclerosis, heart disease, heart attack, ischemia, stroke, nerve damage or poor blood flow in the feet, sepsis-associated encephalopathy (SAE), non-alcoholic steatohepatitis (NASH), non-alcoholic fatty liver disease (NAFLD), end-stage kidney disease, chronic kidney disease, acute renal failure, nephrotic syndrome, renal hyperfiltrative injury, hyperfiltrative diabetic nephropathy, renal hyperfiltration, glomerular hyperfiltration, renal allograft hyperfiltration, compensatory hyperfiltration, hyperfiltrative chronic kidney disease, hyperfiltrative acute renal failure and a measured GFR equal or greater than 125 mL/min/1.73 m². In another embodiment, the present invention is directed to a composition comprising a compound of formula (I) for the treatment of a disorder mediated by GRK2 activity such as obesity, excess weight, impaired glucose tolerance (IGT), impaired fasting glucose (IFT), gestational diabetes, Type II diabetes mellitus, Syndrome X (also known as Metabolic Syndrome), nephropathy, neuropathy, retinopathy, cardiac failure, cardiac hypertrophy, cardiac fibrosis, hypertension, angina, atherosclerosis, heart disease, heart attack, ischemia, stroke, nerve damage or poor blood flow in the feet, sepsis-associated encephalopathy (SAE), non-alcoholic steatohepatitis (NASH), non-alcoholic fatty liver disease (NAFLD), end-stage kidney disease, chronic kidney disease, acute renal failure, nephrotic syndrome, renal hyperfiltrative injury, hyperfiltrative diabetic nephropathy, renal hyperfiltration, glomerular hyperfiltration, renal allograft hyperfiltration, compensatory hyperfiltration, hyperfiltrative chronic kidney disease, hyperfiltrative acute renal failure and a measured GFR equal or greater than 125 mL/min/1.73 m².

Another example of the invention is the use of any of the compounds described herein in the preparation of a medicament for treating: (a) obesity, (b) excess weight, (c) impaired glucose tolerance (IGT), (d) impaired fasting glucose (IFT), (e) gestational diabetes, (f) Type II diabetes mellitus, (g) Syndrome X (also known as Metabolic Syndrome), (h) nephropathy, (i) neuropathy, (j) retinopathy, in a subject in need thereof.

Another example of the invention is the use of any of the compounds described herein in the preparation of a medicament for treating: (a) cardiac failure, (b) cardiac hypertrophy, (c) cardiac fibrosis, (d) hypertension, (e) angina, (f) atherosclerosis, (g) heart disease, (h) heart attack, (i) ischemia, (j) stroke, (k) nerve damage or poor blood flow in the feet and (l) sepsis-associated encephalopathy (SAE), in a subject in need thereof.

Another example of the invention is the use of any of the compounds described herein in the preparation of a medicament for treating: (a) non-alcoholic steatohepatitis (NASH) and (b) non-alcoholic fatty liver disease (NAFLD), in a subject in need thereof.

Another example of the invention is the use of any of the compounds described herein in the preparation of a medicament for treating a disorder as described herein. Another example of the invention is the use of any of the compounds described herein in the preparation of a medicament for treating: (a) end-stage kidney disease, (b) chronic kidney disease, (c) acute renal failure, (d) nephrotic syndrome, (e) renal hyperfiltrative injury, (f) hyperfiltrative diabetic nephropathy, (g) renal hyperfiltration, (h) glomerular hyperfiltration, (i) renal allograft hyperfiltration, (j) compensatory hyperfiltration, (k) hyperfiltrative chronic kidney disease, (l) hyperfiltrative acute renal failure and (m) a measured GFR equal or greater than 125 mL/min/1.73 m², in a subject in need thereof.

In another example, the present invention is directed to a compound as described herein, for use in a method for treating a disorder as described herein. In another example, the present invention is directed to a compound as described herein, for use in a methods for treating a disorder selected from the group consisting of obesity, excess weight, impaired glucose tolerance (IGT), impaired fasting glucose (IFT), gestational diabetes, Type II diabetes mellitus, Syndrome X (also known as Metabolic Syndrome), nephropathy, neuropathy, retinopathy, cardiac failure, cardiac hypertrophy, cardiac fibrosis, hypertension, angina, atherosclerosis, heart disease, heart attack, ischemia, stroke, nerve damage or poor blood flow in the feet, sepsis-associated encephalopathy (SAE), non-alcoholic steatohepatitis (NASH), non-alcoholic fatty liver disease (NAFLD), end-stage kidney disease, chronic kidney disease, acute renal failure, nephrotic syndrome, renal hyperfiltrative injury, hyperfiltrative diabetic nephropathy, renal hyperfiltration, glomerular hyperfiltration, renal allograft hyperfiltration, compensatory hyperfiltration, hyperfiltrative chronic kidney disease, hyperfiltrative acute renal failure and a measured GFR equal or greater than 125 mL/min/1.73 m², in a subject in need thereof.

### DETAILED DESCRIPTION OF THE INVENTION

The present invention is directed to compounds of formula (I)

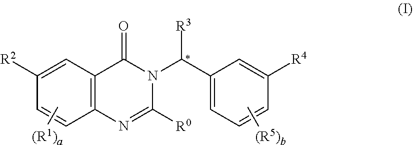

wherein a, b, R⁰, R¹, R², R³, R⁴ and R⁵ are as herein defined, and isotopologues and pharmaceutically acceptable salts thereof.

The compounds of the present invention are useful in the treatment of diseases, disorders and complications associated with GRK2 activity selected from the group consisting of obesity, excess weight, impaired glucose tolerance (IGT), impaired fasting glucose (IFT), gestational diabetes, Type II diabetes mellitus, Syndrome X (also known as Metabolic Syndrome), nephropathy, neuropathy, retinopathy, cardiac failure, cardiac hypertrophy, cardiac fibrosis, hypertension, angina, atherosclerosis, heart disease, heart attack, ischemia, stroke, nerve damage or poor blood flow in the feet, sepsis-associated encephalopathy (SAE), non-alcoholic steatohepatitis (NASH), non-alcoholic fatty liver disease (NAFLD) and renal disorders (including, but not limited to end-stage kidney disease, chronic kidney disease, acute renal failure, nephrotic syndrome, renal hyperfiltrative injury, hyperfiltrative diabetic nephropathy, renal hyperfiltration, glomerular hyperfiltration, renal allograft hyperfiltration, compensatory hyperfiltration, hyperfiltrative chronic kidney disease, hyperfiltrative acute renal failure, a measured GFR equal or greater than 125 mL/min/1.73 m² (for example, a measured GFR equal or greater than 140 mL/min/1.73 m²)).

In an embodiment, the compounds of the present invention are useful in the treatment of diseases, disorders and complications associated with GRK2 activity selected from the group consisting of (a) obesity, (b) excess weight, (c) impaired glucose tolerance (IGT), (d) impaired fasting glucose (IFT), (e) gestational diabetes, (f) Type II diabetes mellitus, (g) Syndrome X (also known as Metabolic Syndrome), (h) nephropathy, (i) neuropathy, (j) retinopathy, (k) cardiac failure, (I) cardiac hypertrophy, (m) cardiac fibrosis, (n) hypertension, (o) angina, (p) atherosclerosis, (q) heart disease, (r) heart attack, (s) ischemia, (t) stroke, (u) nerve damage or poor blood flow in the feet, (v) sepsis-associated encephalopathy (SAE), (w) non-alcoholic steatohepatitis (NASH), (x) non-alcoholic fatty liver disease (NAFLD) (y) kidney disease, (z) chronic kidney disease, (aa) acute renal failure, (ab) nephrotic syndrome, (ac) renal hyperfiltrative injury, (ad) hyperfiltrative diabetic nephropathy, (ae) renal hyperfiltration, (af) glomerular hyperfiltration, (ag) renal allograft hyperfiltration, (ah) compensatory hyperfiltration, (ai) hyperfiltrative chronic kidney disease, (aj) hyperfiltrative acute renal failure and (ak) a measured GFR equal or greater than 125 mL/min/1.73 m².

In an embodiment, the compounds of the present invention are useful in the treatment of diseases, disorders and complications associated with GRK2 activity selected from the group consisting of obesity, excess weight, impaired glucose tolerance (IGT), impaired fasting glucose (IFT), gestational diabetes, Type II diabetes mellitus, Syndrome X (also known as Metabolic Syndrome), diabetic nephropathy, diabetic neuropathy, diabetic retinopathy, cardiac failure, cardiac hypertrophy, hypertension, angina, atherosclerosis, non-alcoholic steatohepatitis (NASH), non-alcoholic fatty liver disease (NAFLD), end-stage kidney disease, chronic kidney disease, acute renal failure, and a measured GFR equal or greater than 125 mL/min/1.73 m².

In another embodiment, the compounds of the present invention are useful in the treatment of diseases, disorders and complications associated with GRK2 activity selected from the group consisting of obesity, excess weight, impaired glucose tolerance (IGT), impaired fasting glucose (IFT), gestational diabetes, Type II diabetes mellitus, Syndrome X (also known as Metabolic Syndrome), diabetic nephropathy, diabetic neuropathy, diabetic retinopathy, non-alcoholic steatohepatitis (NASH), non-alcoholic fatty liver disease (NAFLD), end-stage kidney disease, chronic kidney disease, acute renal failure, and a measured GFR equal or greater than 125 mL/min/1.73 m².

In an embodiment, the compounds of the present invention are useful in the treatment of diseases, disorders and complications associated with GRK2 activity selected from the group consisting of obesity, impaired glucose tolerance (IGT), impaired fasting glucose (IFT), gestational diabetes, Type II diabetes mellitus, Syndrome X (also known as Metabolic Syndrome), diabetic nephropathy, diabetic neuropathy and diabetic retinopathy.

In another embodiment, the compounds of the present invention are useful in the treatment of diseases, disorders and complications associated with GRK2 activity selected from the group consisting of cardiac failure, cardiac hypertrophy, hypertension and atherosclerosis.

In another embodiment, the compounds of the present invention are useful in the treatment of diseases, disorders and complications associated with GRK2 activity selected from the group consisting of non-alcoholic steatohepatitis (NASH) and non-alcoholic fatty liver disease (NAFLD).

In another embodiment, the compounds of the present invention are useful in the treatment of renal diseases, disorders and complications associated with GRK2 activity selected from the group consisting of end-stage kidney disease, chronic kidney disease, acute renal failure, nephrotic syndrome, renal hyperfiltrative injury, hyperfiltrative diabetic nephropathy, renal hyperfiltration, glomerular hyperfiltration, renal allograft hyperfiltration, compensatory hyperfiltration, hyperfiltrative chronic kidney disease, hyperfiltrative acute renal failure and a measured GFR equal or greater than 125 mL/min/1.73 m² (for example, a measured GFR equal or greater than 140 mL/min/1.73 m²)).

In an embodiment of the present invention, R⁰ is selected from the group consisting of hydrogen, C₁₋₄alkyl, fluorinated C₁₋₂alkyl, C₁₋₄alkoxy, fluorinated C₁₋₂alkoxy and 5 to 6 membered, nitrogen containing, saturated heterocyclyl; wherein the 5 to 6 membered, nitrogen containing, saturated heterocyclyl is optionally substituted with one to two substituents independently selected from the group consisting of hydroxy and NR^{X}R^{Y}; wherein R^{X} and R^{Y} are each independently selected from the group consisting of hydrogen and C₁₋₂alkyl;

In another embodiment of the present invention, R⁰ is selected from the group consisting of hydrogen, C₁₋₂alkyl, fluorinated C₁₋₂alkyl and pyrrolidin-1-yl; wherein the pyrrolidin-1-yl is optionally substituted with NR^{X}R^{Y}; wherein R^{X} and R^{Y} are each independently selected from the group consisting of hydrogen and C₁₋₂alkyl.

In another embodiment of the present invention, R⁰ is selected from the group consisting of hydrogen, methyl, trifluoromethyl and 3-amino-pyrrolidin-1-yl. In another embodiment of the present invention, R⁰ is selected from the group consisting of hydrogen, methyl and 3-amino-pyrrolidin-1-yl. In another embodiment of the present invention, R⁰ is hydrogen; In an embodiment of the present invention, a is an integer from 0 to 2.

In another embodiment of the present invention, a is an integer from 0 to 1. In another embodiment of the present invention, a is 0. In another embodiment of the present invention, a is 1. In another embodiment of the present invention a is 2.

In an embodiment of the present invention, each R¹ is independently selected from the group consisting of halogen, hydroxy, C₁₋₂alkyl, fluorinated C₁₋₂alkyl, C₁₋₂alkoxy, fluorinated C₁₋₂alkoxy and cyano. In another embodiment of the present invention, R¹ is selected from the group consisting of halogen, hydroxy, C₁₋₂alkoxy, fluorinated C₁₋₂alkoxy and cyano;

In another embodiment of the present invention, R¹ is selected from the group consisting of 5-hydroxy, 5-chloro, 5-fluoro, 5-methoxy, 5-cyano, 7-fluoro, 7-methoxy and 8-fluoro. In another embodiment of the present invention, R¹ is selected from the group consisting of 5-hydroxy, 5-chloro, 5-fluoro, 5-methoxy, 5-cyano and 7-methoxy. In another embodiment of the present invention, R¹ is selected from the group consisting of 5-hydroxy, 5-fluoro, 5-methoxy and 5-cyano.

In an embodiment of the present invention, R² is selected from the group consisting of 5 to 6 membered heteroaryl and 1H-pyrrolo[2,3-b]pyridin-3-yl; wherein the 5 to 6 membered heteroaryl is optionally substituted with one to two substituents independently selected from the group consisting of halogen, C₁₋₄alkyl, fluorinated C₁₋₂alkyl, oxo and NR^{A}R^{B}; wherein R^{A} and R^{B} are each independently selected from the group consisting of hydrogen and C₁₋₂alkyl.

In another embodiment of the present invention, R² is selected from the group consisting of pyrazolyl, pyrimidinyl, pyridinyl, pyridazinyl, triazolyl, tetrazolyl and 1H-pyrrolo[2,3-b]pyridin-3-yl; wherein the pyrazolyl, pyrimidinyl, pyridinyl, pyridazinyl, triazolyl or tetrazolyl is optionally substituted with a substituent selected from the group consisting of halogen, C₁₋₂alkyl, fluorinated C₁₋₂alkyl, oxo and NR^{A}R^{B}; wherein R^{A} and R^{B} are each independently selected from the group consisting of hydrogen and C₁₋₂alkyl.

In another embodiment of the present invention, R² is selected from the group consisting of pyrazol-4-yl, 3-methyl-pyrazol-4-yl, 3-amino-pyrazol-4-yl, 3-trifluoromethyl-pyrazol-4-yl, pyrimidin-5-yl, 2-amino-pyrimidin-4-yl, pyridin-3-yl, pyridin-4-yl, 6-fluoro-pyridin-3-yl, 1,2,5-triazol-3-yl, 1,2,4-triazol-3-yl-4-one, 1,2,3-5-tetrazol-4-yl, pyridazin-5-yl-3-one and 1H-pyrrolo[2,3-b]pyridin-3-yl. In another embodiment of the present invention, R² is selected from the group consisting of pyrazol-4-yl, 3-methyl-pyrazol-4-yl, 2-amino-pyrazol-4-yl, 1,2,5-triazol-3-yl, 1,2,4-triazol-3-yl-4-one and 1H-pyrrolo[2,3-b]pyridin-3-yl. In another embodiment of the present invention, R² is selected from the group consisting of pyrazol-4-yl, 3-methyl-pyrazol-4-yl and 1,2,5-triazol-3-yl. In another embodiment of the present invention, R² is pyrazol-4-yl.

In an embodiment of the present invention, R³ is selected from the group consisting of hydrogen, —C₁₋₄alkyl, —C₁₋₂alkoxy, —(C₁₋₂alkyl)-OH, —(C₁₋₂alkyl)-NR^{C}R^{D}, —(C₁₋₂alkyl)-SO₂—(C₁₋₂alkyl), —CO₂H, —C(O)O—(C₁₋₂alkyl) and tetrahydropyran-4-yl-1,1-dioxide; wherein R^{C} and R^{D} are each independently selected from the group consisting of hydrogen and C₁₋₂alkyl. In another embodiment of the present invention, R³ is selected from the group consisting of hydrogen, —C₁₋₂alkyl, —(C₁₋₂alkyl)-OH, —(C₁₋₂alkyl)-NR^{C}R^{D}, —(C₁₋₂alkyl)-SO₂—(C₁₋₂alkyl), —CO₂H, —C(O)O—(C₁₋₂alkyl) and tetrahydropyran-4-yl-1,1-dioxide; wherein R^{C} and R^{D} are each independently selected from the group consisting of hydrogen and C₁₋₂alkyl.

In another embodiment of the present invention, R³ is selected from the group consisting of hydrogen, methyl, S-methyl, R-methyl, hydroxymethyl, ethyl, 2-hydroxy-ethyl, —(CH₂CH₂)—NH(CH₃), —(CH₂CH₂)—N(CH₃)₂, —C(O)OH, —C(O)—OCH₃, —CH₂—SO₂—CH₃ and tetrahydro-thiopyran-4-yl 1,1-dioxide. In another embodiment of the present invention, R³ is selected from the group consisting of hydrogen, methyl, R-methyl, ethyl, —CH₂OH, —CH₂CH₂—NH(CH₃) and —CH₂CH₂—N(CH₃)₂. In another embodiment of the present invention, R³ is selected from the group consisting of hydrogen, methyl, R-methyl, —CH₂OH, —CH₂CH₂—NH(CH₃) and —CH₂CH₂—N(CH₃)₂. In another embodiment of the present invention, R³ is selected from the group consisting of hydrogen, methyl, R-methyl, —CH₂OH and —CH₂CH₂—NH(CH₃). In another embodiment of the present invention, R³ is selected from the group consisting of hydrogen, methyl and R-methyl.

In an embodiment of the present invention, R⁴ is selected from the group consisting of hydrogen, halogen, hydroxy, C₁₋₄alkoxy, fluorinated C₁₋₂alkoxy, —O—(C₁₋₂alkyl)-CO₂H, —O—(C₁₋₂alkyl)-C(O)O—(C₁₋₄alkyl), —O—(C₁₋₂alkyl)-C(O)-morpholine, —O—(C₁₋₂alkyl)-C(O)—NR^{E}R^{F}, —(C₁₋₂alkyl)-C(O)—NH—(C₃₋₅cycloalkyl), —O—(C₁₋₂alkyl)-SO₂—(C₁₋₂alkyl), —O—(C₃₋₆cycloalkyl), —O-phenyl, —O-benzyl, —O-azetidin-3-yl, —O-(1-methyl-azetidin-3-yl), —O-pyrrolidin-3-yl, —O-(1-methyl-pyrrolidin-3-yl), —O-piperidin-4-yl, —O-(1-methyl-piperidin-4-yl), —C(O)—(C₁₋₂alkyl), —C(O)—NR^{E}R^{F}, —C(O)—NH-(phenyl), —C(O)—NH-(benzyl), —C(O)—NH—(C₃₋₈cycloalkyl), —C(O)—NH-(pyridinyl), —C(O)—NH—(CH₂CH₂-morpholin-4-yl), —C(O)—NH-(azetidin-3-yl), —C(O)—NH-(1-methyl-azetidin-3-yl), —C(O)—NH-pyrrolidin-3-yl, —C(O)—NH-(1-methyl-pyrrolidin-3-yl), —C(O)—NH-piperidin-4-yl, —C(O)—NH-(1-methyl-piperidin-4-yl), —NH—SO₂—(C₁₋₂alkyl), —S—(C₁₋₄alkyl), —SO—(C₁₋₄alkyl), —SO₂—(C₁₋₄alkyl), —SO₂—NR^{E}R^{F} and oxazol-2-yl; wherein the phenyl or benzyl, whether alone or as part of a substituent group, is optionally substituted with one to two substituents independently selected from the group consisting of halogen, C₁₋₂alkyl and C₁₋₂alkoxy; and wherein R^{E} and R^{F} are each independently selected form the group consisting of hydrogen and C₁₋₂alkyl.

In another embodiment of the present invention, R⁴ is selected from the group consisting of hydrogen, halogen, hydroxy, C₁₋₂alkoxy, fluorinated C₁₋₂alkoxy, —O—(C₁₋₂alkyl)-C(O)OH, —O—(C₁₋₂alkyl)-C(O)O—(C₁₋₂alkyl), —O—(C₁₋₂alkyl)-C(O)—NH—(C₃₋₅cycloalkyl), —O—(C₁₋₂alkyl)-SO₂—(C₁₋₂alkyl), —O-phenyl, —O-benzyl, —O-azetidin-3-yl, —O-(1-methyl-azetidin-3-yl), —C(O)—(C₁₋₂alkyl), —C(O)—NH-(phenyl), —C(O)—NH-(benzyl), —C(O)—NH-(azetidin-3-yl), —C(O)—NH-(1-methyl-azetidin-3-yl), —NH—SO₂—(C₁₋₂alkyl), and oxazol-2-yl; wherein the phenyl or benzyl, whether alone or as part of a substituent group, is optionally substituted with halogen.

In another embodiment of the present invention, R⁴ is selected from the group consisting of hydrogen, chloro, fluoro, hydroxy, methoxy, ethoxy, difluoromethoxy, trifluoromethoxy, —O—CH₂—C(O)OH, —O—CD₂-C(O)OH, —O—CD₂-C(O)—NH(cyclopropyl), —O—CH₂CH₂—SO₂—CH₃, —O-(4-fluorophenyl), —O-benzyl, —O-(1-methyl-azetidin-3-yl), —C(O)—CH₃, —C(O)—NH-(4-fluorobenzyl), —C(O)—NH-(2,6-difluorobenzyl), —C(O)—NH-(1-methyl-azetidin-3-yl), —NH—SO₂—CH₃, —SO₂—NH₂, and oxazol-2-yl. In another embodiment of the present invention, R⁴ is selected from the group consisting of hydrogen, chloro, hydroxy, methoxy, ethoxy, difluoromethoxy, —O—CH₂—C(O)OH, —O—CD₂-C(O)OH, —O—CD₂-C(O)—NH(cyclopropyl), —O-(4-fluorophenyl), —O-benzyl, —C(O)—CH₃, —C(O)—NH-(4-fluorobenzyl), —C(O)—NH-(2,6-difluorobenzyl), —C(O)—NH-(1-methyl-azetidin-3-yl), —NH—SO₂—CH₃, —SO₂—NH₂ and oxazol-2-yl.

In another embodiment of the present invention, R⁴ is selected from the group consisting of hydrogen, chloro, hydroxy, methoxy, ethoxy, —O—CH₂—C(O)OH, —O—CD₂-C(O)—NH(cyclopropyl), —O-(4-fluorophenyl), —O-benzyl, —C(O)—CH₃, —C(O)—NH-(4-fluorobenzyl), —C(O)—NH-(2,6-difluorobenzyl), —C(O)—NH-(1-methyl-azetidin-3-yl), —NH—SO₂—CH₃ and —SO₂—NH₂. In another embodiment of the present invention, R⁴ is selected from the group consisting of chloro, hydroxy, methoxy, ethoxy, —O—CD₂-C(O)—NH(cyclopropyl), —C(O)—NH-(4-fluorobenzyl), —C(O)—NH-(2,6-difluorobenzyl), —NH—SO₂—CH₃ and —SO₂—NH₂. In another embodiment of the present invention, R⁴ is selected from the group consisting of hydroxy, methoxy, —C(O)—NH-(4-fluorobenzyl) and —C(O)—NH-(2,6-difluorobenzyl).

In an embodiment of the present invention, b is an integer from 0 to 2. In another embodiment of the present invention, b is an integer from 0 to 1. In another embodiment of the present invention, b is 0. In another embodiment of the present invention, b is 1. In another embodiment of the present invention, b is 2.

In an embodiment of the present invention, each R⁵ is independently selected from the group consisting of halogen, C₁₋₂alkyl and C₁₋₄alkoxy. In another embodiment of the present invention, each R⁵ is independently selected from the group consisting of halogen and C₁₋₂alkoxy.

In another embodiment of the present invention, (R⁵)_{b} is selected from the group consisting of selected from the group consisting of 4-fluoro, 4-methoxy, 5-fluoro, 6-fluoro and 6-methoxy and 2,6-difluoro. In another embodiment of the present invention, (R⁵)_{b} is selected from the group consisting of selected from the group consisting of 4-fluoro, 5-fluoro and 2,6-difluoro. In another embodiment of the present invention, (R⁵)_{b} is selected from the group consisting of selected from the group consisting of 5-fluoro and 2,6-difluoro.

In another embodiment, the present invention is directed to a compound of formula (I) selected from the group consisting of
3-[(3-methoxyphenyl)methyl]-4-oxo-6-(1H-pyrazol-4-yl)quinazoline-5-carbonitrile;
5-fluoro-3-[(3-methoxyphenyl)methyl]-6-(1H-pyrazol-4-yl)quinazolin-4-one;
3-[(1R)-1-(3-methoxyphenyl)ethyl]-6-(1H-pyrazol-4-yl)quinazolin-4-one;
5-methoxy-3-[(3-methoxyphenyl)methyl]-6-(1H-pyrazol-4-yl)quinazolin-4-one;
3-[3-(dimethylamino)-1-(3-methoxyphenyl)propyl]-6-(1H-pyrazol-4-yl)quinazolin-4-one;
N-(4-fluorophenyl)-3-[[4-oxo-6-(1H-pyrazol-4-yl)quinazolin-3-yl]methyl]benzamide;
N-[(2,6-difluorophenyl)methyl]-3-[[4-oxo-6-(1H-pyrazol-4-yl)quinazolin-3-yl]methyl]benzamide;
N-[(4-fluorophenyl)methyl]-3-[[4-oxo-6-(1H-pyrazol-4-yl)quinazolin-3-yl]methyl]benzamide;

and isomers and pharmaceutically acceptable salts thereof.

In an embodiment of the present invention, when R⁰ is hydrogen or methyl; a is an integer from 0 to 1; R¹, when present, is 8-methyl; R³ is hydrogen, R⁴ is methoxy, and b is 0, then R² is other than pyrazol-4-yl or imidazol-1-yl. In another embodiment of the present invention, when R⁰ is hydrogen or methyl; a is an integer from 0 to 1; R¹, when present, is 8-methyl; R³ is hydrogen, R⁴ is methoxy, and b is 0, then R² is other than pyrazol-4-yl.

Additional embodiments of the present invention, include those wherein the substituents selected for one or more of the variables defined herein (i.e. a, b, R⁰, R¹, R², R³, R⁴ and R⁵, etc.) are independently selected to be any individual substituent or any subset of substituents selected from the complete list as defined herein. Additional embodiments of the present invention, include those wherein the substituents selected for one or more of the variables defined herein (i.e. a, b, R⁰, R¹, R², R³, R⁴ and R⁵, etc.) are independently selected to correspond to any of the embodiments as defined herein.

In another embodiment of the present invention is any single compound or subset of compounds selected from the representative compounds listed in Table 1, below.

Representative compounds of the present invention are as listed in Table 1, below. Unless otherwise noted, wherein a stereogenic center is present in the listed compound, the compound was prepared as a mixture of stereo-configurations.

TABLE 1
Representative Compounds of Formula (I)

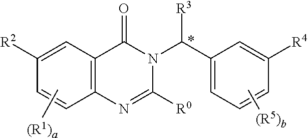

| ID No. | (R¹)ₐ | R² | R³ | R⁰ | R⁴ | (R⁵)_{b} |
|---|---|---|---|---|---|---|
| 1 | 5-cyano | pyrazol-4-yl | H | H | methoxy | b = 0 |
| 2 | a = 0 | pyrazol-4-yl | H | H | methoxy | b = 0 |
| 3 | 5-fluoro | pyrazol-4-yl | H | H | methoxy | b = 0 |
| 4 | 5-hydroxy | pyrazol-4-yl | H | H | methoxy | b = 0 |
| 5 | 5-methoxy | pyrazol-4-yl | H | H | methoxy | b = 0 |
| 6 | 7-methoxy | pyrazol-4-yl | H | H | methoxy | b = 0 |
| 7 | 5-chloro | pyrazol-4-yl | H | H | methoxy | b = 0 |
| 8 | 5-fluoro | pyrazol-4-yl | H | H | ethoxy | b = 0 |
| 9 | 8-fluoro | pyrazol-4-yl | H | H | methoxy | b = 0 |
| 10 | a = 0 | 2-amino- | H | methyl | methoxy | b = 0 |
| | | pyrimidin-4- | | | | |
| | | yl | | | | |
| 11 | a = 0 | pyrazol-4-yl | methoxy- | H | methoxy | b = 0 |
| | | | carbonyl | | | |
| 12 | a = 0 | pyrazol-4-yl | carboxy | H | methoxy | b = 0 |
| 13 | a = 0 | pyrazol-4-yl | H | H | (4-fluoro- | b = 0 |
| | | | | | benzyl)- | |
| | | | | | amino- | |
| | | | | | carbonyl | |
| 14 | a = 0 | pyrazol-4-yl | S-methyl | H | methoxy | b = 0 |
| 15 | a = 0 | pyrazol-4-yl | methyl- | H | ethoxy | b = 0 |
| | | | sulfonyl- | | | |
| | | | methyl | | | |
| 17 | a = 0 | pyrazol-4-yl | methyl | H | methyl- | b = 0 |
| | | | | | sulfonyl- | |
| | | | | | ethoxy- | |
| 19 | a = 0 | 3-amino- | methyl | H | methoxy | b = 0 |
| | | pyrazol-4-yl | | | | |
| 20 | a = 0 | pyrazol-4-yl | H | methyl | methoxy | b = 0 |
| 21 | a = 0 | pyrazol-4-yl | H | trifluoro- | methoxy | b = 0 |
| | | | | methyl | | |
| 22 | a = 0 | pyrazol-4-yl | tetrahydro- | H | methoxy | b = 0 |
| | | | thiopyran- | | | |
| | | | 4-yl 1,1- | | | |
| | | | dioxide | | | |
| 23 | a = 0 | pyrazol-4-yl | R-methyl | H | cycloprop- | b = 0 |
| | | | | | yl-amino- | |
| | | | | | carbonyl- | |
| | | | | | d2- | |
| | | | | | methoxy- | |
| 24 | a = 0 | pyrazol-4-yl | methyl | H | (1-methyl- | b = 0 |
| | | | | | azetidin- | |
| | | | | | 3-yl)- | |
| | | | | | amino- | |
| | | | | | carbonyl | |
| 25 | a = 0 | pyrazol-4-yl | H | methyl | (1-methyl- | b = 0 |
| | | | | | azetidin- | |
| | | | | | 3-yl)oxy | |
| 26 | a = 0 | pyrazol-4-yl | R-methyl | H | carboxy- | b = 0 |
| | | | | | d2- | |
| | | | | | methoxy | |
| 27 | a = 0 | pyrazol-4-yl | H | H | carboxy- | b = 0 |
| | | | | | methoxy | |
| 28 | a = 0 | pyrazol-4-yl | H | H | oxazol-2- | b = 0 |
| | | | | | yl | |
| 29 | a = 0 | pyrazol-4-yl | dimethyl- | H | methoxy | b = 0 |
| | | | amino- | | | |
| | | | ethyl | | | |
| 30 | a = 0 | pyrazol-4-yl | methyl- | H | methoxy | b = 0 |
| | | | amino- | | | |
| | | | ethyl | | | |
| 31 | a = 0 | pyrazol-4-yl | methyl | H | hydroxy | b = 0 |
| 32 | a = 0 | pyrazol-4-yl | 2-hydroxy- | H | methoxy | b = 0 |
| | | | ethyl | | | |
| 33 | a = 0 | pyrazol-4-yl | methyl | H | methyl- | b = 0 |
| | | | | | sulfonyl- | |
| | | | | | amino | |
| 34 | a = 0 | pyrazol-4-yl | H | H | amino- | b = 0 |
| | | | | | sulfonyl | |
| 35 | a = 0 | pyrazol-4-yl | ethyl | H | methoxy | b = 0 |
| 37 | a = 0 | 3-methyl- | H | H | methoxy | b = 0 |
| | | pyrazol-4-yl | | | | |
| 38 | a = 0 | 1,2,4-triazol- | H | H | methoxy | b = 0 |
| | | 3-yl-4-one | | | | |
| 39 | a = 0 | pyrazol-4-yl | R-methyl | H | methoxy | b = 0 |
| 40 | a = 0 | pyrazol-4-yl | H | H | fluoro | 6- |
| | | | | | | methoxy |
| 41 | a = 0 | pyrazol-4-yl | H | H | methyl- | b = 0 |
| | | | | | carbonyl | |
| 42 | a = 0 | pyrazol-4-yl | hydroxy- | H | methoxy | b = 0 |
| | | | methyl | | | |
| 43 | a = 0 | 1H- | H | H | methoxy | b = 0 |
| | | pyrrolo[2,3- | | | | |
| | | b]pyridin-3-yl | | | | |
| 44 | a = 0 | 1,2,5-triazol- | H | H | methoxy | b = 0 |
| | | 3-yl | | | | |
| 45 | a = 0 | 2-amino- | H | H | methoxy | b = 0 |
| | | pyrimidin-4- | | | | |
| | | yl | | | | |
| 46 | a = 0 | pyridazin-5- | H | H | methoxy | b = 0 |
| | | yl-3-one | | | | |
| 48 | a = 0 | 1,2,3-5- | H | H | methoxy | b = 0 |
| | | tetrazol-4-yl | | | | |
| 49 | a = 0 | pyridin-3-yl | H | H | chloro | b = 0 |
| 50 | a = 0 | pyrazol-4-yl | H | H | chloro | b = 0 |
| 52 | a = 0 | 2-amino- | H | H | methoxy | b = 0 |
| | | pyrimidin-4- | | | | |
| | | yl | | | | |
| 53 | a = 0 | 1H- | H | H | methoxy | b = 0 |
| | | pyrrolo[2,3- | | | | |
| | | b]pyridin-3-yl | | | | |
| 54 | a = 0 | 1H- | H | H | methoxy | 4-fluoro |
| | | pyrrolo[2,3- | | | | |
| | | b]pyridin-3-yl | | | | |
| 55 | a = 0 | pyrazol-4-yl | H | 3-amino- | (4-fluoro- | b = 0 |
| | | | | pyrrolidin- | benzyl)- | |
| | | | | 1-yl | amino- | |
| | | | | | carbonyl | |
| 57 | a = 0 | pyrazol-4-yl | H | H | (4-fluoro- | b = 0 |
| | | | | | benzyl)- | |
| | | | | | amino- | |
| | | | | | carbonyl | |
| 60 | a = 0 | pyrazol-4-yl | H | H | (4-fluoro- | b = 0 |
| | | | | | benzyl)- | |
| | | | | | amino- | |
| | | | | | carbonyl | |
| 61 | a = 0 | pyrazol-4-yl | methyl | H | (4-fluoro- | b = 0 |
| | | | | | benzyl)- | |
| | | | | | amino- | |
| | | | | | carbonyl | |
| 62 | a = 0 | pyrazol-4-yl | H | H | methoxy | 4-fluoro |
| 63 | a = 0 | pyrazol-4-yl | H | H | benzyloxy | b = 0 |
| 64 | a = 0 | pyrazol-4-yl | H | H | difluoro- | b = 0 |
| | | | | | methoxy | |
| 65 | a = 0 | pyrazol-4-yl | H | H | 4-fluoro- | b = 0 |
| | | | | | phenyloxy | |
| 66 | a = 0 | pyrazol-4-yl | H | H | trifluoro- | b = 0 |
| | | | | | methoxy | |
| 67 | a = 0 | pyrazol-4-yl | H | H | fluoro | 4- |
| | | | | | | methoxy |
| 68 | 8-fluoro | pyrazol-4-yl | H | H | H | b = 0 |
| 69 | 7-fluoro | pyrazol-4-yl | H | H | H | b = 0 |
| 70 | a = 0 | pyrazol-4-yl | H | H | (2,6- | 2,6- |
| | | | | | difluoro- | difluoro |
| | | | | | benzyl)- | |
| | | | | | amino- | |
| | | | | | carbonyl | |
| 72 | a = 0 | pyrazol-4-yl | H | H | (2,6- | 5-fluoro |
| | | | | | difluoro- | |
| | | | | | benzyl)- | |
| | | | | | amino- | |
| | | | | | carbonyl | |
| 77 | a = 0 | 3-trifluoro- | H | H | H | 2,6- |
| | | methyl- | | | | difluoro |
| | | pyrazol-4-yl | | | | |
| 78 | a = 0 | pyrazol-4-yl | H | H | H | 2,6- |
| | | | | | | difluoro |
| 79 | a = 0 | pyrazol-4-yl | H | H | H | b = 0 |
| 80 | a = 0 | pyrazol-4-yl | H | H | H | b = 0 |
| 81 | a = 0 | pyridin-4-yl | H | H | H | b = 0 |
| 82 | a = 0 | 6-fluoro- | H | H | H | b = 0 |
| | | pyridin-3-yl | | | | |
| 83 | a = 0 | pyrimidin-5- | H | H | H | b = 0 |
| | | yl | | | | |
| 84 | a = 0 | pyrazol-4-yl | H | H | H | b = 0 |

The present invention is further directed to one or more compounds selected from the group consisting of
N-(4-fluorobenzyl)-3-(4-oxo-6-(1H-pyrazol-4-yl)quinazolin-3(4H)-yl)benzamide;
N-(2,4-difluorobenzyl)-4-((4-oxo-6-(1H-pyrrol-3-yl)quinazolin-3(4H)-yl)methyl)benzamide;
N-(2,6-difluorobenzyl)-4-((4-oxo-6-(1H-pyrrol-3-yl)quinazolin-3(4H)-yl)methyl)benzamide;
3-(benzo[d][1,3]dioxol-4-ylmethyl)-6-(1H-pyrazol-4-yl)quinazolin-4(3H)-one;
N-(4-fluorobenzyl)-4-((4-oxo-6-(1H-pyrazol-4-yl)quinazolin-3(4H)-yl)methyl)picolinamide;
3-(3-methoxybenzyl)-6-(2-((2-methoxyethyl)amino)pyrimidin-4-yl)quinazolin-4(3H)-one;

and isomers and pharmaceutically acceptable salts thereof.

### Definitions

As used herein, unless otherwise noted, “halogen” shall mean chloro, bromo, fluoro and iodo, preferably bromo, fluoro or chloro.

As used herein, unless otherwise noted, the term “oxo” shall mean s functional group of the structure ═O (i.e. a substituent oxygen atom connected to another atom by a double bond).

As used herein, unless otherwise noted, the term “C_{X-Y}alkyl” wherein X and Y are integers, whether used alone or as part of a substituent group, include straight and branched chains containing between X and Y carbon atoms. For example, C₁₋₄alkyl radicals include straight and branched chains of between 1 and 4 carbon atoms, including methyl, ethyl, propyl, isopropyl, butyl, isobutyl, sec-butyl and t-butyl.

One skilled in the art will recognize that the terms “—(C_{X-Y}alkyl)- and —C_{X-Y}alkyl-” wherein X and Y are integers, shall denote any C_{X-Y}alkyl carbon chain as herein defined, wherein said C_{X-Y}alkyl chain is divalent and is further bound through two points of attachment, preferably through two terminal carbon atoms.

As used herein, unless otherwise noted, the term “fluorinated C_{X-Y}alkyl” shall mean any C_{X-Y}alkyl group as defined above substituted with at least one fluorine atom, preferably one to three fluorine atoms. In an example, “fluorinated C₁₋₄alkyl” include, but are not limited, to —CH₂F, —CF₂H, —CF₃, —CH₂—CF₃, —CF₂—CF₂—CF₂—CF₃, and the like.

As used herein, unless otherwise noted, “C_{X-Y}alkynyl” wherein X and Y are integers, shall mean any straight or branched chain of between X and Y carbon atoms, wherein the straight or branched chain contains as least one, preferably one, unsaturated double bond. For example, the term “C₂₋₆alkynyl” includes straight and branched chains of between 2 and 6 carbon atoms containing at least one, preferably one, unsaturated double bond such as ethynyl, n-propyn-1-yl, n-butyn-1-yl, n-but-2-yn-1-yl, n-but-1-yn-2-yl, pentyn-1-yl, pent-2-yn-1-yl, and the like.

As used herein, unless otherwise noted, “C_{X-Y}alkoxy” wherein X and Y are integers, shall mean an oxygen ether radical of the above described straight or branched chain C_{X-Y}alkyl groups containing between X and Y carbon atoms. For example, C₁₋₄alkoxy shall include methoxy, ethoxy, n-propoxy, isopropoxy, n-butyloxy, iso-butyloxy, sec-butyloxy and tert-butyloxy.

As used herein, unless otherwise noted, the term “fluorinated C_{X-Y}alkoxy” shall mean any C_{X-Y}alkoxy group as defined above substituted with at least one fluorine atom, preferably one to three fluorine atoms. For example, “fluorinated C₁₋₄alkoxy” include, but are not limited, —OCH₂F, —OCF₂H, —OCF₃, —OCH₂—CF₃, —OCF₂—CF₂—CF₂—CF₃, and the like.

As used herein, unless otherwise noted, the term “C_{X-Y}cycloalkyl”, wherein X and Y are integers, shall mean any stable X- to Y-membered monocyclic, bicyclic, polycyclic, bridged or spiro-cyclic saturated ring system, preferably a monocyclic, bicyclic, bridged or spiro-cyclic saturated ring system. For example, the term “C₃₋₈cycloalkyl” includes, but is not limited to cyclopropyl, cyclobutyl, cyclopentyl, cyclohexyl, cycloheptyl, bicyclo[2.2.1]hept-2-yl, cyclooctyl, bicyclo[2.2.2]octan-2-yl, and the like.

As used herein, unless otherwise noted, the term “5 to 6 membered heteroaryl” shall denote any five or six membered monocyclic aromatic ring structure containing at least one heteroatom selected from the group consisting of O, N and S, optionally containing one to three additional heteroatoms independently selected from the group consisting of O, N and S. The heteroaryl group may be attached at any heteroatom or carbon atom of the ring such that the result is a stable structure. Examples of suitable 5 to 6 membered heteroaryl groups include, but are not limited to, pyrrolyl, furyl, thienyl, oxazolyl, imidazolyl, purazolyl, isoxazolyl, isothiazolyl, triazolyl, thiadiazolyl, pyridyl, pyridazinyl, pyrimidinyl, pyrazinyl, pyranyl, furazanyl, and the like.

As used herein, unless otherwise noted, the term “4 to 7 membered nitrogen containing, saturated heterocyclyl” shall mean and saturated, monocyclic 4 to 7 membered ring structure, wherein at least one of the 4 to 7 ring atoms is a nitrogen. Said 4 to 7 membered nitrogen containing, saturated heterocyclyl may optionally contain one or more additional heteroatoms independently selected from the group consisting of N, O and S (wherein the S is optionally substituted with one or two oxo groups). Suitably examples include, but are not limited to azetidinyl, pyrrolidinyl, piperidinyl, piperazinyl, morpholinyl, thiomorpholinyl, dihydropyrimidinyl, triazinanyl, oxadiazinanyl, and the like. Preferably, the 4 to 7 membered nitrogen containing, saturated heterocyclyl is one or more selected from the group consisting of azetidinyl, pyrrolidinyl, piperidinyl, piperazinyl and morpholinyl.

When a particular group is “substituted” (e.g. C_{X-Y}alkyl, C_{X-Y}alkoxy, C_{X-Y}cycloalkyl, etc.), that group may have one or more substituents, preferably from one to five substituents, more preferably from one to three substituents, most preferably from one to two substituents, independently selected from the list of substituents.

With reference to substituents, the term “independently” means that when more than one of such substituents is possible, such substituents may be the same or different from each other.

As used herein, the notation “*” shall denote the presence of a stereogenic center.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, wherein the compound is present as a diastereomer, the diastereomer is present at an diastereomeric excess of greater than or equal to about 80%, more preferably, at an diastereomeric excess of greater than or equal to about 90%, more preferably still, at an diastereomeric excess of greater than or equal to about 95%, more preferably still, at an diastereomeric excess of greater than or equal to about 98%, most preferably, at an diastereomeric excess of greater than or equal to about 99%.

Furthermore, some of the crystalline forms for the compounds of the present invention may exist as polymorphs and as such are intended to be included in the present invention. In addition, some of the compounds of the present invention may form solvates with water (i.e., hydrates) or common organic solvents, and such solvates are also intended to be encompassed within the scope of this invention.

As used herein, unless otherwise noted, the term “isotopologues” shall mean molecules that differ only in their isotopic composition. More particularly, an isotopologue of a molecule differs from the parent molecule in that it contains at least one atom which is an isotope (i.e. has a different number of neutrons from its parent atom).

For example, isotopologues of water include, but are not limited to, “light water” (HOH or H₂O), “semi-heavy water” with the deuterium isotope in equal proportion to protium (HDO or ¹H²HO), “heavy water” with two deuterium isotopes of hydrogen per molecule (D₂O or ²H₂O), “super-heavy water” or tritiated water (T₂O or ³H₂O), where the hydrogen atoms are replaced with tritium (³H) isotopes, two heavy-oxygen water isotopologues (H₂¹⁸O and H₂¹⁷O) and isotopologues where the hydrogen and oxygen atoms may each independently be replaced by isotopes, for example the doubly labeled water isotopologue D₂¹⁸O.

It is intended that within the scope of the present invention, any one or more element(s), in particular when mentioned in relation to a compound of formula (I), shall comprise all isotopes and isotopic mixtures of said element(s), either naturally occurring or synthetically produced, either with natural abundance or in an isotopically enriched form. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O. The isotopes may be radioactive or non-radioactive. Radiolabelled compounds of formula (I) may comprise one or more radioactive isotope(s) selected from the group of ³H, ¹¹C, ¹⁸F, ¹²²I, ¹²³I, ¹²⁵I, ¹³¹I, ⁷⁵Br, ⁷⁶Br, ⁷⁷Br and ⁸²Br. Preferably, the radioactive isotope is selected from the group of ³H, ¹¹C and ¹⁸F.

As used herein, unless otherwise noted, the term “isotopomers” shall mean isomers with isotopic atoms, having the same number of each isotope of each element but differing in their position. Isotopomers include both constitutional isomers and stereoisomers solely based on isotopic location. For example, CH₃CHDCH₃ and CH₃CH₂CH₂D are a pair of constitutional isotopomers of n-propane; whereas (R)—CH₃CHDOH and (S)—CH₃CHDOH or (Z)—CH₃CH═CHD and (E)-CH₃CH═CHD are examples of isotopic stereoisomers of ethanol and n-propene, respectively.

It is further intended that the present invention includes the compounds described herein, including all isomers thereof (including, but not limited to stereoisomers, enantiomers, diastereomers, tautomers, isotopologues, isotopomers, and the like).

Under standard nomenclature used throughout this disclosure, the terminal portion of the designated side chain is described first, followed by the adjacent functionality toward the point of attachment. Thus, for example, a “phenylC₁-C₆alkylaminocarbonylC₁-C₆alkyl” substituent refers to a group of the formula

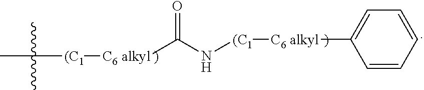

Abbreviations used in the specification, particularly the Schemes and Examples, are as listed in the Table A, below:

TABLE A
Abbreviations

| | | |
|---|---|---|
| ADDP | = | 1,1′-(Azodicarbonyl)dipiperidine |
| ADP | = | Adenosine Diphosphate |
| Alexa633 tracer | = | Alexa Fluor ® 633 Hydrazide Tracer |
| | | (Available from ThermoFisher) |
| BSA | = | Bovine Serum Albumin |
| ACN or MeCN | = | Acetonitrile |
| ATP | = | Adenosine Triphosphate |
| Boc or BOC | = | tert-Butoxyloxycarbonyl (i.e. -C(O)-O-C(CH₃)₃) |
| Brij ^{™}-35 | = | Polyethylene glycol hexadecyl ether |
| DCM | = | Dichloromethane |
| DEAD | = | Diethyl Azodicarboxylate |
| DIAD | = | Diisopropyl Azodicarboxylate |
| DIPEA or DIEA | = | Diisopropylethylamine |
| DME | = | Dimethoxyethane |
| DMF | = | N,N-Dimethylformamide |
| DMSO | = | Dimethylsulfoxide |
| dppf | = | 1,1′-Bis(diphenylphosphino)ferrocene |
| DTT | = | Dithiothietol |
| EA or EtOAc | = | Ethyl Acetate |
| EDCI | = | 1-Ethyl-3-(3-dimethylaminopropyl)carbodiimide |
| EDTA | = | Ethylenediaminetetracetic acid |
| eGFR | = | Estimated Glomular Filtration Rate |
| ES | = | ElectroSpray (mass spectroscopy) |
| Et₃N or TEA | = | Triethylamine |
| EtOAc or EA | = | Ethyl acetate |
| F12 medium | = | Gibco ® F12 Nutrient Medium |
| | | (Available from ThermoFisher) |
| FBS | = | Fetal Bovine Serum |
| G418 | = | Geneticin ® (G418) Sulfate |
| GFR | = | Glomular Filtration Rate |
| GRK2 | = | G protein-coupled Receptor Kinase 2 |
| HATU | = | (1-[Bis(dimethylamino)methylene-1H-1,2,3- |
| | | triazolo[4,5-b]pyridinium 3-oxid |
| | | hexafluorophosphate |
| HBSS | = | GIBCO ® Hank's Balanced Salt Solution |
| HEPES | = | 4-(2-Hydroxyethyl)-1-Piperizine Ethane Sulfonic |
| | | Acid |
| HOBt or HOBT | = | Hydroxybenzotriazole |
| HPLC | = | High Pressure Liquid Chromatography |
| HTRF | = | Homogeneous Time Resolved Fluorescence |
| IFG | = | Impaired fasting glucose |
| IGT | = | Impaired glucose tolerance |
| LC-MS or LC/MS | = | Liquid chromatography-masss spectrometry |
| mCPBA | = | meta-Chloroperoxybenzoic acid |
| MeCN or ACN | = | Acetonitrile |
| MeOH | = | Methanol |
| mesyl or Ms | = | Methylsulfonyl (i.e. -SO2-CH₃) |
| mesylate or OMs | = | Methanesulfonate (i.e. -O-SO₂-CH₃) |
| MOM | = | Methoxy methyl |
| Ms or mesyl | = | -SO₂-CH₃ |
| MTBE | = | Methyl tert-Butyl Ether |
| NAFLD | = | Non-alcoholic fatty liver disease |
| NaOt-Bu | = | Sodium tert-Butoxide |
| NASH | = | Non-alcoholic steatohepatitis |
| NMR | = | Nuclear Magnetic Resonance |
| OGTT | = | Oral Glucose Tolerance Test |
| OMs or mesylate | = | Methanesulfonate (i.e. -O-SO₂-CH₃) |
| OTf or triflate | = | Trifluoromethanesulfonyl (i.e. -O-SO₌-CF₃) |
| OTs or tosylate | = | p-Toluenesulfonate (i.e. -O-SO₂-(p-methylphenyl)) |
| Pd(dba)₃ | = | Tris(dibenzylideneacetone)dipalladium(0) |
| Pd(dppf)Cl₂ | = | [1,1′-Bis(diphenylphosphino)ferrocene] Palladium |
| | | (II) Dichloride |
| Pd(PPh₃)₄ | = | Tetrakistriphenylphosphine palladium (0) |
| PE | = | Petroleum ether |
| PPh₃ | = | Triphenylphosphine |
| SAE | = | Sepsis Associated Encephalopathy |
| TBAF | = | Tetra-n-butylammonium fluoride |
| TBDMS | = | tert-Butyldimethylsilyl |
| TEA | = | Triethylamine |
| Tf or trifyl | = | Trifluoromethylsulfonyl (i.e. -SO₂-CF₃) |
| TFA | = | Trifluoroacetic Acid |
| THF | = | Tetrahydrofuran |
| THP | = | Tetrahydropyran |
| TLC | = | Thin Layer Chromatography |
| TMS | = | Trimethylsilyl |
| Tosylate or OTs | = | p-Toluenesulfonate (i.e. -O-SO₂-(p-methylphenyl)) |
| Ts or tosyl | = | -SO₂-(p-methylphenyl) |
| Tween-20 ® | = | Nonionic detergent (Sigma Aldrich) |

As used herein, unless otherwise noted, the term “isolated form” shall mean that the compound is present in a form which is separate from any solid mixture with another compound(s), solvent system or biological environment.

In an embodiment of the present invention, the compound of formula (I) is present in an isolated form. In another embodiment of the present invention, the compound of formula (C1), (C2), (C3), (C4), (C5) or (C6) is present in an isolated form.

As used herein, unless otherwise noted, the term “substantially pure form” shall mean that the mole percent of impurities in the isolated compound is less than about 5 mole percent, preferably less than about 2 mole percent, more preferably, less than about 0.5 mole percent, most preferably, less than about 0.1 mole percent. In an embodiment of the present invention, the compound of formula (I) is present as a substantially pure form. In another embodiment of the present invention, the compound of formula (C1), (C2), (C3), (C4), (C5) or (C6) is present as a substantially pure form.

As used herein, unless otherwise noted, the term “substantially free of a corresponding salt form(s)” when used to described the compound of formula (I) shall mean that mole percent of the corresponding salt form(s) in the isolated base of formula (I) is less than about 5 mole percent, preferably less than about 2 mole percent, more preferably, less than about 0.5 mole percent, most preferably less than about 0.1 mole percent. In an embodiment of the present invention, the compound of formula (I) is present in a form which is substantially free of corresponding salt form(s). In another embodiment of the present invention, the compound of formula (C1), (C2), (C3), (C4), (C5) or (C6) is present in a form which is substantially free of corresponding salt form(s).

As used herein, unless otherwise noted, the terms “treating”, “treatment” and the like, shall include the management and care of a subject or patient (preferably mammal, more preferably human) for the purpose of combating a disease, condition, or disorder and includes the administration of a compound of the present invention to prevent the onset of the symptoms or complications, alleviate the symptoms or complications, slow the progression of the disease or disorder, or eliminate the disease, condition, or disorder.

As used herein, unless otherwise noted, the term “prevention” shall include (a) reduction in the frequency of one or more symptoms; (b) reduction in the severity of one or more symptoms; (c) the delay or avoidance of the development of additional symptoms; and/or (d) delay or avoidance of the development of the disorder or condition.

One skilled in the art will recognize that wherein the present invention is directed to methods of prevention, a subject in need of thereof (i.e. a subject in need of prevention) shall include any subject or patient (preferably a mammal, more preferably a human) who has experienced or exhibited at least one symptom of the disorder, disease or condition to be prevented. Further, a subject in need thereof may additionally be a subject (preferably a mammal, more preferably a human) who has not exhibited any symptoms of the disorder, disease or condition to be prevented, but who has been deemed by a physician, clinician or other medical profession to be at risk of developing said disorder, disease or condition. For example, the subject may be deemed at risk of developing a disorder, disease or condition (and therefore in need of prevention or preventive treatment) as a consequence of the subject's medical history, including, but not limited to, family history, pre-disposition, co-existing (comorbid) disorders or conditions, genetic testing, and the like.

The term “subject” as used herein, refers to an animal, preferably a mammal, most preferably a human, who has been the object of treatment, observation or experiment. Preferably, the subject has experienced and/or exhibited at least one symptom of the disease or disorder to be treated and/or prevented.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

As more extensively provided in this written description, terms such as “reacting” and “reacted” are used herein in reference to a chemical entity that is any one of: (a) the actually recited form of such chemical entity, and (b) any of the forms of such chemical entity in the medium in which the compound is being considered when named.

One skilled in the art will recognize that, where not otherwise specified, the reaction step(s) is performed under suitable conditions, according to known methods, to provide the desired product. One skilled in the art will further recognize that, in the specification and claims as presented herein, wherein a reagent or reagent class/type (e.g. base, solvent, etc.) is recited in more than one step of a process, the individual reagents are independently selected for each reaction step and may be the same of different from each other. For example wherein two steps of a process recite an organic or inorganic base as a reagent, the organic or inorganic base selected for the first step may be the same or different than the organic or inorganic base of the second step. Further, one skilled in the art will recognize that wherein a reaction step of the present invention may be carried out in a variety of solvents or solvent systems, said reaction step may also be carried out in a mixture of the suitable solvents or solvent systems.

One skilled in the art will recognize that wherein a reaction step of the present invention may be carried out in a variety of solvents or solvent systems, said reaction step may also be carried out in a mixture of the suitable solvents or solvent systems.

One skilled in the art will further recognize that the reaction or process step(s) as herein described are allowed to proceed for a sufficient period of time until the reaction is complete, as determined by any method known to one skilled in the art, for example, chromatography (e.g. HPLC). In this context a “completed reaction or process step” shall mean that the reaction mixture contains a significantly diminished amount of the starting material(s)/reagent(s) and a significantly reduced amount of the desired product(s), as compared to the amounts of each present at the beginning of the reaction.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any amount or range therein.

Examples of suitable solvents, bases, reaction temperatures, and other reaction parameters and components are provided in the detailed descriptions which follow herein. One skilled in the art will recognize that the listing of said examples is not intended, and should not be construed, as limiting in any way the invention set forth in the claims which follow thereafter.

As used herein, unless otherwise noted, the term “leaving group” shall mean a charged or uncharged atom or group which departs during a substitution or displacement reaction. Suitable examples include, but are not limited to, Br, Cl, I, mesylate, tosylate, and the like.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

As used herein, unless otherwise noted, the term “nitrogen protecting group” shall mean a group which may be attached to a nitrogen atom to protect said nitrogen atom from participating in a reaction and which may be readily removed following the reaction. Suitable nitrogen protecting groups include, but are not limited to carbamates—groups of the formula —C(O)O—R wherein R is for example methyl, ethyl, t-butyl, benzyl, phenylethyl, CH₂═CH—CH₂—, and the like; amides—groups of the formula —C(O)—R′ wherein R′ is for example methyl, phenyl, trifluoromethyl, and the like; N-sulfonyl derivatives—groups of the formula —SO₂—R″ wherein R″ is for example tolyl, phenyl, trifluoromethyl, 2,2,5,7,8-pentamethylchroman-6-yl-, 2,3,6-trimethyl-4-methoxybenzene, and the like. Other suitable nitrogen protecting groups may be found in texts such as T. W. Greene & P. G. M. Wuts, Protective Groups in Organic Synthesis, John Wiley & Sons, 1991.

As used herein, unless otherwise noted, the term “oxygen protecting group” shall mean a group which may be attached to an oxygen atom to protect said oxygen atom from participating in a reaction and which may be readily removed following the reaction. Suitable oxygen protecting groups include, but are not limited to, acetyl, benzoyl, t-butyl-dimethylsilyl, trimethylsilyl (TMS), MOM, THP, and the like. Other suitable oxygen protecting groups may be found in texts such as T. W. Greene & P. G. M. Wuts, Protective Groups in Organic Synthesis, John Wiley & Sons, 1991.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

Additionally, chiral HPLC against a standard may be used to determine percent enantiomeric excess (% ee). The enantiomeric excess may be calculated as follows

[(Rmoles−Smoles)/(Rmoles+Smoles)]×100%

where Rmoles and Smoles are the R and S mole fractions in the mixture such that Rmoles+Smoles=1. The enantiomeric excess may alternatively be calculated from the specific rotations of the desired enantiomer and the prepared mixture as follows:

ee=([α−obs]/[α−max])×100.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include, but are not limited to, the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids which may be used in the preparation of pharmaceutically acceptable salts include, but are not limited to, the following: acids including acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, a-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid and undecylenic acid.

Representative bases which may be used in the preparation of pharmaceutically acceptable salts include, but are not limited to, the following: bases including ammonia, L-arginine, benethamine, benzathine, calcium hydroxide, choline, decanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

### General Synthesis Schemes

Compounds of formula (I) of the present invention may be synthesized according to the general synthesis schemes described below. The preparation of the various starting materials used in the synthesis schemes which follow hereinafter is well within the skill of persons versed in the art.

Compounds of formula (I) may be prepared as described in Scheme 1, below.

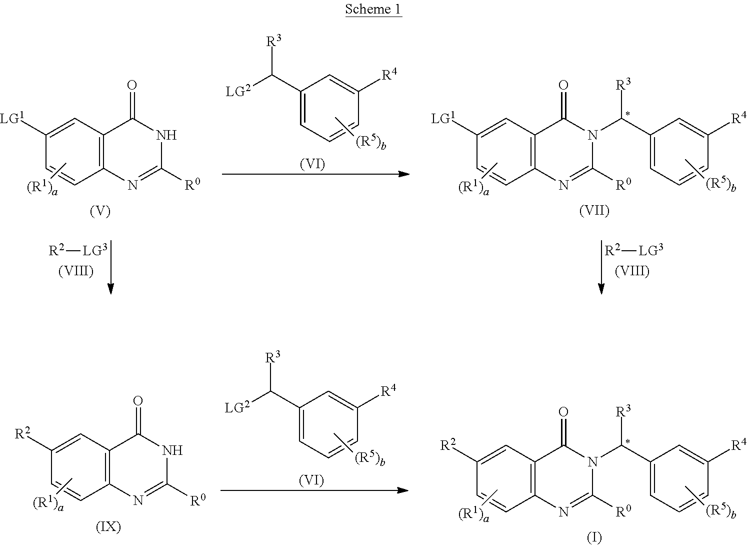

Accordingly, a suitably substituted compound of formula (V), wherein LG¹ is a suitably selected leaving group such as Br, I, OTf, and the like, a known compound or compound prepared by known methods, is reacted with a suitably substituted compound of formula (VI), wherein LG² is a suitably selected leaving group such as Br, Cl, OTf, OH, and the like, a known compound or compound prepared by known methods; under suitably selected coupling conditions; for example, the coupling conditions may be (a) in the presence of a suitably selected base such as Cs₂CO₃, K₂CO₃, NaH, NaOt-Bu, TEA, and the like, in a suitably selected solvent such as DMF, acetonitrile, 1,4-dioxane, THF, and the like; or (b) under Mitsunobu reaction conditions using reagents such as PPh₃, DEAD, DIAD, ADDP, and the like, in a suitably selected solvent such as THF, toluene, acetonitrile, and the like; to yield the corresponding compound of formula (VII).

The compound of formula (VII) is reacted with a suitably substituted compound of formula (VIII), wherein LG³ is a suitably selected leaving group such as boronic acid, tributyltin, boronic ester, and the like; in the presence of a suitably selected base such as K₂CO₃, Cs₂CO₃, K₃PO₄, NaOt-Bu, and the like; in the presence of a suitably selected catalyst such as Pd(PPh₃)₄, Pd(dppf)Cl₂, and the like; in a suitably selected solvent such as 1,4-dioxane, toluene, DMF, and the like; to yield the corresponding compound of formula (I).

Alternatively, a suitably substituted compound of formula (V) wherein LG¹ is a suitably selected leaving group such as Br, Cl, OMs, and the like, a known compound or compound prepared by known methods, is reacted with a suitably substituted compound of formula (VIII), wherein LG³ is a suitably selected leaving group such as boronic acid, tributyltin, boronic ester, and the like, a known compound or compound prepared by known methods; in the presence of a suitably selected base such as K₂CO₃, Cs₂CO₃, K₃PO₄, NaOt-Bu, and the like; in the presence of a suitably selected catalyst such as Pd(PPh₃)₄, Pd(dppf)Cl₂, and the like; in a suitably selected solvent such as 1,4-dioxane, toluene, DMF, and the like; to yield the corresponding compound of formula (IX).

The compound of formula (IX) is reacted with a suitably substituted compound of formula (VI), wherein LG² is a suitably selected leaving group such as Br, Cl, OTs, and the like, a known compound or compound prepared by known methods; under suitably selected coupling conditions; for example, the coupling conditions may be (a) in the presence of a suitably selected base such as Cs₂CO₃, K₂CO₃, NaH, NaOt-Bu, TEA, and the like, in a suitably selected solvent such as DMF, acetonitrile, 1,4-dioxane, THF, and the like; or (b) under Mitsunobu reaction conditions using reagents such as PPh₃, DEAD, DIAD, ADDP, and the like, in a suitably selected solvent such as THF, toluene, acetonitrile, and the like; to yield the corresponding compound of formula (I).

One skilled in the art will recognize, that the R⁰ group on the compound of formula (V) and/or compound of formula (VII) may be optionally and/or preferably protected with a suitably selected protecting group (for example, wherein R⁰ is an anime, the amine is preferably protected with a suitably selected nitrogen protecting group such as Boc, and the like) prior to the reaction of the compound of formula (V) or compound of formula (VII) with the suitably substituted compound of formula (VIII), and then de-protected, according to known methods (for example where the protecting group is Boc by reacting with a suitably selected acid such as HCl), at a suitably step thereafter.

Compounds of formula (I), wherein R³ is —(C₁₋₂alkyl)-NR^{C}R^{D}, may alternatively be prepared as described in Scheme 2, below.

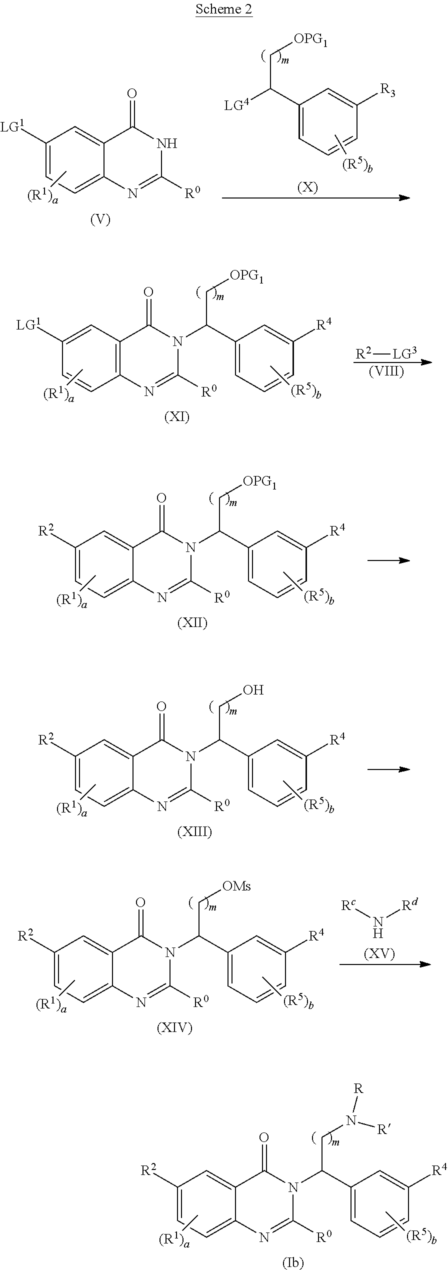

Accordingly, a suitably substituted compound of formula (V), wherein LG¹ is a suitably selected leaving group such as Br, I, OTf, and the like, a known compound or compound prepared by known methods, is reacted with a suitably substituted compound of formula (X), wherein LG⁴ is a suitably selected leaving group such as Br, Cl, OTf, OH, and the like, and wherein PG¹ is a suitably selected oxygen protecting group such as TMS, TBDMS, benzyl, and the like, a known compound or compound prepared by known methods; under suitably selected coupling conditions; for example, the coupling conditions may be (a) in the presence of a suitably selected base such as Cs₂CO₃, K₂CO₃, NaH, NaOt-Bu, TEA, and the like, in a suitably selected solvent such as DMF, acetonitrile, 1,4-dioxane, THF, and the like; or (b) under Mitsunobu reaction conditions using reagents such as PPh₃, DEAD, DIAD, ADDP, and the like, in a suitably selected solvent such as THF, toluene, acetonitrile, and the like; to yield the corresponding compound of formula (XI).

The compound of formula (XI) is reacted with a suitably substituted compound of formula (VIII), wherein LG³ is a suitably selected leaving group such as boronic acid, tributyltin, boronic ester, and the like; in the presence of a suitably selected base such as K₂CO₃, Cs₂CO₃, K₃PO₄, NaOt-Bu, and the like; in the presence of a suitably selected catalyst such as Pd(PPh₃)₄, Pd(dppf)Cl₂, and the like; in a suitably selected solvent such as 1,4-dioxane, toluene, DMF, and the like; to yield the corresponding compound of formula (XII).

The compound of formula (XII) is reacted to remove the PG¹ protecting group, according to known methods; to yield the corresponding compound of formula (XIII). For example, wherein the PG¹ oxygen protecting group is TBDMS, the compound of formula (XII) is de-protected by reacting with TBAF or pyridine.HF.

The compound of formula (XIII) is reacted with mesyl chloride (as shown in the Scheme above, or alternatively with tosyl chloride), a known compound; in the presence of a suitably selected base such as TEA, DIPEA, pyridine, N-methylmorpholine, and the like; in a suitably selected solvent such as DCM, CHCl₃, THF, and the like; to yield the corresponding compound of formula (XIV).

The compound of formula (XIV) is reacted with a suitably substituted compound of formula (XV), a known compound or compound prepared by known methods; in a suitably selected solvent such as THF, acetonitrile, DMF, and the like; to yield the corresponding compound of formula (Ia).

One skilled in the art will recognize, that the R⁰ and/or R² group(s) may be optionally and/or preferably protected with a suitably selected protecting group prior to any reaction step in which said groups contain a reactive group, and then then de-protected, according to known methods, at any subsequent, suitably reaction step thereafter, to yield the desired compound of formula (I).

For example, wherein R⁰ is an anime, the amine is preferably protected with a suitably selected nitrogen protecting group such as Boc, benzyl, and the like, and then de-protected, by reacting with a suitably selected acid such as HCl, TFA, BBr₃, pyridine.HCl, and the like. In another example, wherein R² is pyrazol-4-yl, the nitrogen at the 1-position is preferably protected with a suitably selected nitrogen protecting group such as tetrahydropyran-2-yl, Boc, and the like, and then de-protected, by reacting with a suitably selected acid such as TFA, HCl, TsOH, and the like.

The compound of formula (D) may be prepared as described in Scheme 3, below.

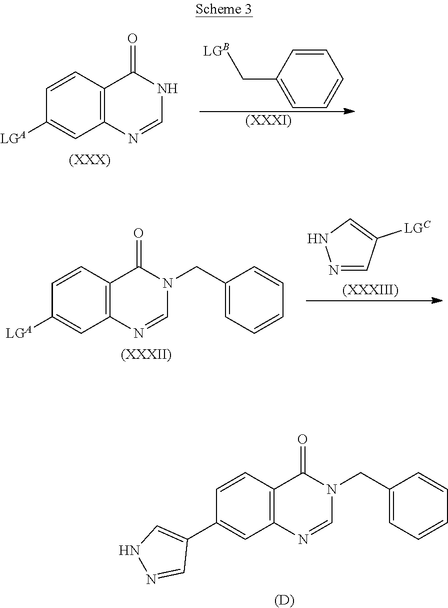

Accordingly, a suitably substituted compound of formula (XXX) wherein LG^{A} is a suitably selected leaving group such as Br, Cl, OMs, and the like, a known compound or compound prepared by known methods, is reacted with a suitably substituted compound of formula (XXXI), wherein LG^{B} is a suitably selected leaving group such as Br, Cl, OTs, and the like, a known compound or compound prepared by known methods; under suitably selected coupling conditions; for example, the coupling conditions may be (a) in the presence of a suitably selected base such as Cs₂CO₃, K₂CO₃, NaH, NaOt-Bu, TEA, and the like, in a suitably selected solvent such as DMF, acetonitrile, 1,4-dioxane, THF, and the like; or (b) under Mitsunobu reaction conditions using reagents such as PPh₃, DEAD, DIAD, ADDP, and the like, in a suitably selected solvent such as THF, toluene, acetonitrile, and the like; to yield the corresponding compound of formula (XXXII).

The compound of formula (XXXII) is reacted with a suitably substituted compound of formula (XXXIII), wherein LG^{C} is a suitably selected leaving group such as boronic acid, tributyltin, boronic ester, and the like, a known compound or compound prepared by known methods; in the presence of a suitably selected base such as K₂CO₃, Cs₂CO₃, K₃PO₄, NaOt-Bu, and the like; in the presence of a suitably selected catalyst such as Pd(PPh₃)₄, Pd(dppf)Cl₂, and the like; in a suitably selected solvent such as 1,4-dioxane, toluene, DMF, and the like; to yield the corresponding compound of formula (D).

### Pharmaceutical Compositions

The present invention further comprises pharmaceutical compositions containing one or more compounds of formula (I) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus, for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like, for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.01 mg to about 1000 mg or any amount or range therein, and may be given at a dosage of from about 0.05 mg/day to about 300 mg/day, or any amount or range therein, preferably from about 0.1 mg/day to about 100 mg/day, or any amount or range therein, preferably from about 1 mg/day to about 50 mg/day, or any amount or range therein. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from about 0.01 mg to about 1,000 mg, or any amount or range therein, of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating disorders mediated by GRK2 activity, described in the present invention may also be carried out using a pharmaceutical composition comprising any of the compounds as defined herein and a pharmaceutically acceptable carrier. The pharmaceutical composition may contain between about 0.01 mg and about 1000 mg of the compound, or any amount or range therein, preferably from about 0.05 mg to about 300 mg of the compound, or any amount or range therein, more preferably from about 0.1 mg to about 100 mg of the compound, or any amount or range therein, more preferably from about 0.1 mg to about 50 mg of the compound, or any amount or range therein, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

To prepare a pharmaceutical composition of the present invention, a compound of formula (I) as the active ingredient is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

Compounds of this invention may be administered in any of the foregoing compositions and according to dosage regimens established in the art whenever treatment of disorders mediated by GRK2 activity, is required.

The daily dosage of the products may be varied over a wide range from about 0.01 mg to about 1,000 mg per adult human per day, or any amount or range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the patient to be treated. An effective amount of the drug may be ordinarily supplied at a dosage level of from about 0.005 mg/kg to about 10 mg/kg of body weight per day, or any amount or range therein. Preferably, the range is from about 0.01 to about 5.0 mg/kg of body weight per day, or any amount or range therein, more preferably, from about 0.1 to about 1.0 mg/kg of body weight per day, or any amount or range therein, more preferably, from about 0.1 to about 0.5 mg/kg of body weight per day, or any amount or range therein. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

The following Examples are set forth to aid in the understanding of the invention, and are not intended and should not be construed to limit in any way the invention set forth in the claims which follow thereafter.

In the Examples which follow, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.

### Example 1: Compound #70

### N-(2,6-Difluorobenzyl)-3-((4-oxo-6-(1H-pyrazol-4-yl)quinazolin-3(4H)-yl)methyl)benzamide

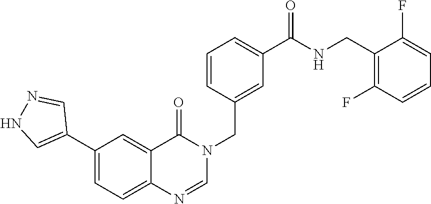 [SMILES: O=C(NCc1c(F)cccc1F)c1cccc(Cn2cnc3ccc(-c4cn[nH]c4)cc3c2=O)c1]

To a solution of 3-((6-bromo-4-oxoquinazolin-3(4H)-yl)methyl)-N-(2,6-difluorobenzyl)benzamide (40 mg, 0.083 mmol, 1 equiv) in DMF (1 ml) and water (1 ml) was successively added K₂CO₃ (34.413 mg, 0.249 mmol, 3 equiv), tert-butyl 4-(4,4,5,5-tetramethyl-1,3,2-dioxaborolan-2-yl)-1H-pyrazole-1-carboxylate (24.415 mg, 0.083 mmol, 1 equiv) and Pd(PPh₃)₄ (9.591 mg, 0.008 mmol, 0.1 equiv). The resulting mixture was degassed with N₂ and stirred at 100° C. overnight. The reaction mixture was concentrated under reduced pressure and the residue was purified by flash column chromatography on silica gel to yield tert-butyl 4-(3-(3-(2,6-difluorobenzylcarbamoyl)benzyl)-4-oxo-3,4-dihydroquinazolin-6-yl)-1H-pyrazole-1-carboxylate.

To solution of tert-butyl 4-(3-(3-(2,6-difluorobenzylcarbamoyl)benzyl)-4-oxo-3,4-dihydroquinazolin-6-yl)-1H-pyrazole-1-carboxylate (40 mg, 0.070 mmol, 1 equiv) in DCM (1 ml) was added TFA (0.1 ml, 0.982 mmol, 14 equiv). After stirring for 1 h, the reaction was quenched with saturated NaHCO₃ (aq), then extracted with DCM. The combined organic phase was dried over anhydrous Na₂SO₄, filtered and concentrated to yield N-(2,6-difluorobenzyl)-3-((4-oxo-6-(1H-pyrazol-4-yl)quinazolin-3(4H)-yl)methyl)benzamide as an off-white solid.

¹H NMR (400 MHz, METHANOL-d₄) δ 8.40 (brd, J=12.13 Hz, 2H), 8.08 (br d, J=8.59 Hz, 3H), 7.82 (br s, 1H), 7.72 (br d, J=5.56 Hz, 2H), 7.54-7.61 (m, 1H), 7.41-7.49 (m, 1H), 7.26-7.37 (m, 1H), 6.89-7.01 (m, 2H), 5.30 (s, 2H), 4.63 (br s, 2H). m/z (MH⁺): 472.20

### Example 2: Compound #2

### 3-(3-Methoxybenzyl)-6-(1H-pyrazol-4-yl)quinazolin-4(3H)-one

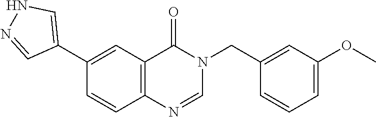 [SMILES: COc1cccc(Cn2cnc3ccc(-c4cn[nH]c4)cc3c2=O)c1]

A mixture of 6-bromo-3-(3-methoxybenzyl)quinazolin-4(3H)-one (500 mg, 1.448 mmol, 1.00 equiv), tert-butyl 4-(4,4,5,5-tetramethyl-1,3,2-dioxaborolan-2-yl)-1H-pyrazole-1-carboxylate (639 mg, 2.173 mmol, 1.50 equiv), potassium carbonate (400 mg, 2.897 mmol, 2.00 equiv) and tetrakis(triphenylphosphine)palladium(0) (167 mg, 0.145 mmol, 0.10 equiv) in 1,4-dioxane (8 mL) and water (2 mL) was stirred at 100° C. overnight. After filtration, the filtrate was diluted with water and extracted with EtOAc twice. The combined organic layer was washed with brine, dried over Na₂SO₄ and concentrated. The resulting residue was re-crystallized from diethyl ether/DCM in the ratio of 5:1 to yield 3-(3-methoxybenzyl)-6-(1H-pyrazol-4-yl)quinazolin-4(3H)-one as a light yellow solid.

¹H NMR (300 MHz, DMSO-d6) d 13.03 (s, 1H), 8.47 (s, 1H), 8.35 (s, 1H), 8.28 (d, J=2.1 Hz, 1H), 8.05 (td, J=7.7, 7.0, 2.7 Hz, 2H), 7.64 (d, J=8.5 Hz, 1H), 7.22 (t, J=7.9 Hz, 1H), 6.79-6.97 (m, 3H), 5.14 (s, 2H), 3.69 (s, 3H). m/z (MH⁺): 333.0.

### Example 3: Compound #29

### 3-(3-(Dimethylamino)-1-(3-methoxyphenyl)propyl)-6-(1H-pyrazol-4-yl)quinazolin-4(3H)-one

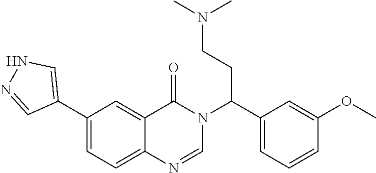 [SMILES: COc1cccc(C(CCN(C)C)n2cnc3ccc(-c4cn[nH]c4)cc3c2=O)c1]

A solution of 3-(3-methoxyphenyl)-3-(4-oxo-6-(1-(tetrahydro-2H-pyran-2-yl)-1H-pyrazol-4-yl)quinazolin-3(4H)-yl)propyl methanesulfonate (200 mg, 0.371 mmol, 1.00 equiv) and the solution of dimethylamine in THE (2.0 M, 3 mL, 6 mmol) was stirred at 80° C. overnight. The solvent was removed under reduced pressure and the residue was purified by preparative TLC (MeOH/DCM=10%) to yield 3-(3-(dimethylamino)-1-(3-methoxyphenyl)propyl)-6-(1-(tetrahydro-2H-pyran-2-yl)-1H-pyrazol-4-yl)quinazolin-4(3H)-one as a yellow solid.

To a solution of 3-(3-(dimethylamino)-1-(3-methoxyphenyl)propyl)-6-(1-(tetrahydro-2H-pyran-2-yl)-1H-pyrazol-4-yl)quinazolin-4(3H)-one (60 mg, 0.123 mmol, 1.00 equiv) in dichloromethane (1 mL) was added trifluoroacetic acid (0.5 mL) at 0° C. The solution was stirred for 3 h at room temperature. The solvent was removed under reduced pressure and the residue was purified by reversal column chromatography on C18 (40 g, MeCN/H₂O (0.05% CF₃COOH): 0>>>40%) to yield 3-(3-(dimethylamino)-1-(3-methoxyphenyl)propyl)-6-(1H-pyrazol-4-yl)quinazolin-4(3H)-one as an off-white solid.

¹H NMR (300 MHz, DMSO-d6) d 9.62 (s, 1H), 8.40 (s, 1H), 8.32 (d, J=2.1 Hz, 1H), 8.22 (s, 2H), 8.11 (dd, J=8.5, 2.1 Hz, 1H), 7.67 (d, J=8.5 Hz, 1H), 7.33 (t, J=7.9 Hz, 1H), 7.00-7.11 (m, 2H), 6.94 (dd, J=8.2, 2.4 Hz, 1H), 6.02 (t, J=7.7 Hz, 1H), 3.76 (s, 3H), 3.08-3.18 (m, 2H), 2.69-2.90 (m, 8H). ¹⁹F NMR (282 MHz, DMSO-d6) d −73.85-−74.20 (m). m/z (MH⁺): 404.1

### Example 4: Compound #30

### 3-(1-(3-Methoxyphenyl)-3-(methylamino)propyl)-6-(1H-pyrazol-4-yl)quinazolin-4(3H)-one

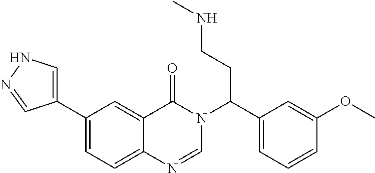 [SMILES: CNCCC(c1cccc(OC)c1)n1cnc2ccc(-c3cn[nH]c3)cc2c1=O]

A solution of 6-bromo-3-(3-(tert-butyldimethylsilyloxy)-1-(3-methoxyphenyl)propyl)quinazolin-4(3H)-one (1.5 g, 2.979 mmol, 1.00 equiv), 1-(tetrahydro-2H-pyran-2-yl)-4-(4,4,5,5-tetramethyl-1,3,2-dioxaborolan-2-yl)-1H-pyrazole (0.994 g, 3.575 mmol, 1.20 equiv), tetrakis(triphenylphosphine)palladium(0) (344 mg, 0.298 mmol, 0.10 equiv) and potassium carbonate (1.235 g, 8.937 mmol, 3.00 equiv) in DMF (20 mL) and water (2 mL) was stirred at 100° C. overnight under atmosphere of nitrogen. After filtration, the filtrate was diluted with water and extracted with EtOAc twice. The combined organic layer was washed with brine, dried over Na₂SO₄ and concentrated. The residue was purified by flash column chromatography on silica gel (80 g, EtOAc/PE: 0>>>60%) to yield 3-(3-(tert-butyldimethylsilyloxy)-1-(3-methoxyphenyl)propyl)-6-(1-(tetrahydro-2H-pyran-2-yl)-1H-pyrazol-4-yl)quinazolin-4(3H)-one as a brown oil.

A solution of 3-(3-(tert-butyldimethylsilyloxy)-1-(3-methoxyphenyl)propyl)-6-(1-(tetrahydro-2H-pyran-2-yl)-1H-pyrazol-4-yl)quinazolin-4(3H)-one (800 mg, 1.392 mmol, 1.00 equiv) and tetrabutylammonium fluoride (2M, 7 mL, 7 mmol, 5.00 equiv) in THE (15 mL) was stirred at room temperature overnight. After diluting with water, the solution was extracted with EtOAc twice. The combined organic layer was washed with brine, dried over Na₂SO₄ and concentrated. The residue was purified by flash column chromatography on silica gel (80 g, MeOH/DCM: 0>>>10%) to yield 3-(3-hydroxy-1-(3-methoxyphenyl)propyl)-6-(1-(tetrahydro-2H-pyran-2-yl)-1H-pyrazol-4-yl)cinnolin-4(3H)-one as a white solid.

To a solution of 3-(3-hydroxy-1-(3-methoxyphenyl)propyl)-6-(1-(tetrahydro-2H-pyran-2-yl)-1H-pyrazol-4-yl)cinnolin-4(3H)-one (300 mg, 0.651 mmol, 1.00 equiv) and triethylamine (99 mg, 0.977 mmol, 1.50 equiv) in DCM (10 mL) was added to methylsulfochloride (90 mg, 0.782 mmol, 1.20 equiv) at 0° C., portion-wise. The suspension was stirred at room temperature for 2 h. The suspension was poured into water and extracted with EtOAc twice. The combined organic layer was washed with brine, dried over Na₂SO₄ and concentrated. The residue was purified by flash column chromatography on silica gel (120 g, EtOAc/PE: 0>>>30%) to yield 3-(3-methoxyphenyl)-3-(4-oxo-6-(1-(tetrahydro-2H-pyran-2-yl)-1H-pyrazol-4-yl)quinazolin-3(4H)-yl)propyl methanesulfonate as a white solid.

A solution of 3-(3-methoxyphenyl)-3-(4-oxo-6-(1-(tetrahydro-2H-pyran-2-yl)-1H-pyrazol-4-yl)quinazolin-3(4H)-yl)propyl methanesulfonate (100 mg, 0.186 mmol, 1.00 equiv) and methylamine/MeOH (2 M, 3 mL) was stirred at 80° C. for 3 h. The solvent was removed, and the residue was purified by flash column chromatography on silica gel (40 g, EtOAc/PE: 0>>>30%) to yield 3-(1-(3-methoxyphenyl)-3-(methylamino)propyl)-6-(1-(tetrahydro-2H-pyran-2-yl)-1H-pyrazol-4-yl)quinazolin-4(3H)-one as a white solid.

To a solution of 3-(1-(3-methoxyphenyl)-3-(methylamino)propyl)-6-(1-(tetrahydro-2H-pyran-2-yl)-1H-pyrazol-4-yl)quinazolin-4(3H)-one (60 mg, 0.127 mmol, 1.00 equiv) in dichloromethane (1 mL) was added trifluoroacetic acid (0.5 mL) at 0° C. The solution was stirred for 3 h at room temperature. The solvent was removed, and the residue was purified by reversal column chromatography on C18 (40 g, MeCN/H₂O (0.05% CF₃COOH): 0>>>40%) to yield 3-(1-(3-methoxyphenyl)-3-(methylamino)propyl)-6-(1H-pyrazol-4-yl)quinazolin-4(3H)-one as an off-white solid.

¹H NMR (300 MHz, DMSO-d6) d 8.51 (s, 2H), 8.39 (s, 1H), 8.31 (d, J=2.1 Hz, 1H), 8.22 (s, 2H), 8.11 (dd, J=8.5, 2.1 Hz, 1H), 7.67 (d, J=8.5 Hz, 1H), 7.33 (t, J=8.1 Hz, 1H), 6.99-7.09 (m, 2H), 6.90-6.97 (m, 1H), 6.00 (t, J=7.8 Hz, 1H), 3.75 (s, 3H), 2.94 (dt, J=13.0, 6.4 Hz, 2H), 2.52-2.75 (m, 5H). ¹⁹F NMR (282 MHz, DMSO-d6) d −74.26; m/z (MH⁺): 390.0

### Example 5: Compound #32

### 3-(3-Hydroxy-1-(3-methoxyphenyl)propyl)-6-(1H-pyrazol-4-yl)quinazolin-4(3H)-one

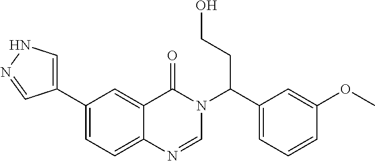 [SMILES: COc1cccc(C(CCO)n2cnc3ccc(-c4cn[nH]c4)cc3c2=O)c1]

To a solution of 3-(3-(tert-butyldimethylsilyloxy)-1-(3-methoxyphenyl)propyl)-6-(1-(tetrahydro-2H-pyran-2-yl)-1H-pyrazol-4-yl)quinazolin-4(3H)-one (100 mg, 0.174 mmol, 1.00 equiv) in dichloromethane (2 mL) was added trifluoroacetic acid (1 mL) at 0° C. The solution was stirred overnight at room temperature. The solvent was removed under reduced pressure and the residue was stirred with K₂CO₃ (120 mg, 0.870 mmol, 5.00 equiv) and MeOH (2 mL) at room temperature for 3 h. After filtration, the filtrate was concentrated under reduced pressure and the residue was purified by reversal column chromatography on C18 (40 g, MeCN/H₂O: 0>>>60%) to yield 3-(3-Hydroxy-1-(3-methoxyphenyl)propyl)-6-(1H-pyrazol-4-yl)quinazolin-4(3H)-one as a white solid.

¹H NMR (400 MHz, DMSO-d6) d 13.04 (s, 1H), 8.50 (s, 1H), 8.25-8.40 (m, 2H), 8.00-8.08 (m, 2H), 7.64 (d, J=8.5 Hz, 1H), 7.27 (t, J=8.1 Hz, 1H), 7.00-7.07 (m, 2H), 6.86 (ddd, J=8.3, 2.5, 1.0 Hz, 1H), 6.08 (t, J=8.0 Hz, 1H), 4.64 (t, J=4.8 Hz, 1H), 3.73 (s, 3H), 3.40 (q, J=5.7 Hz, 2H), 2.39-2.57 (m, 2H). m/z (MH⁺): 377.2

### Example 6: Compound #13

### N-(4-Fluorobenzyl)-3-((4-oxo-6-(1H-pyrazol-4-yl)quinazolin-3(4H)-yl)methyl)benzamide

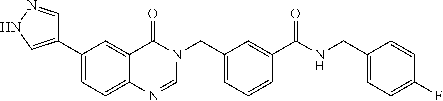 [SMILES: O=C(NCc1ccc(F)cc1)c1cccc(Cn2cnc3ccc(-c4cn[nH]c4)cc3c2=O)c1]

Under an inert atmosphere of nitrogen, a mixture of 3-((6-bromo-4-oxoquinazolin-3(4H)-yl)methyl)-N-(4-fluorobenzyl)benzamide (300 mg, 0.643 mmol, 1.00 equiv), tert-butyl 4-(4,4,5,5-tetramethyl-1,3,2-dioxaborolan-2-yl)-1H-pyrazole-1-carboxylate (284 mg, 0.965 mmol, 1.50 equiv), K₂CO₃ (267 mg, 1.932 mmol, 3.00 equiv) and Pd(PPh₃)₄ (37 mg, 0.032 mmol, 0.05 equiv) in DMF (15 mL) and H₂O (1.5 mL) was stirred at 110° C. for 3 h. The resulting mixture was then cooled and diluted with EtOAc (100 mL), washed with brine (4×30 mL), dried over Na₂SO₄, and concentrated. The residue was recrystallized with EtOAc to yield N-(4-fluorobenzyl)-3-((4-oxo-6-(1H-pyrazol-4-yl)quinazolin-3(4H)-yl)methyl)benzamide as white solid.

¹H NMR (400 MHz, METHANOL-d₄) δ 8.42 (s, 1H), 8.36 (d, J=2.02 Hz, 1H), 8.06 (dd, J=2.02, 8.59 Hz, 3H), 7.88 (s, 1H), 7.78 (d, J=8.08 Hz, 1H), 7.68 (d, J=8.59 Hz, 1H), 7.59 (d, J=7.58 Hz, 1H), 7.44-7.50 (m, 1H), 7.32 (dd, J=5.31, 8.34 Hz, 2H), 6.97-7.06 (m, 2H), 5.31 (s, 2H), 4.50 (s, 2H). m/z (MH⁺): 454.20

### Example 7: Compound #14

### (S)-3-(1-(3-Methoxyphenyl)ethyl)-6-(1H-pyrazol-4-yl)quinazolin-4(3H)-one

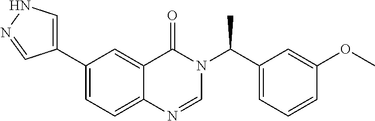 [SMILES: COc1cccc([C@H](C)n2cnc3ccc(-c4cn[nH]c4)cc3c2=O)c1]

Under an inert atmosphere of nitrogen, a mixture of (S)-6-bromo-3-(1-(3-methoxyphenyl)ethyl)quinazolin-4(3H)-one (120 mg, 0.334 mmol, 1.00 equiv), tert-butyl 4-(4,4,5,5-tetramethyl-1,3,2-dioxaborolan-2-yl)-1H-pyrazole-1-carboxylate (295 mg, 1.003 mmol, 3.00 equiv), K₂CO₃ (139 mg, 1.006 mmol, 3.00 equiv) and Pd(PPh₃)₄ (19 mg, 0.016 mmol, 0.05 equiv) in DMF (10 mL) with H₂O (1 mL) was stirred for 3 h at 100° C. EtOAc (100 mL) was added, and the resulting mixture was washed with water, brine, dried over Na₂SO₄, and concentrated. The resulting residue was applied onto a silica gel column (40 g, MeOH:DCM/0>>>5%>>>10%) to yield (S)-3-(1-(3-methoxyphenyl)ethyl)-6-(1H-pyrazol-4-yl)quinazolin-4(3H)-one as white solid.

¹H NMR (400 MHz, DMSO-d6) d 8.37 (s, 2H), 8.32 (d, J=2.1 Hz, 1H), 8.10 (dd, J=8.5, 2.2 Hz, 2H), 7.66 (d, J=8.5 Hz, 1H), 7.29 (t, J=7.9 Hz, 1H), 6.93-7.03 (m, 2H), 6.85-6.93 (m, 1H), 6.10 (q, J=7.2 Hz, 1H), 3.75 (s, 3H), 1.85 (d, J=7.2 Hz, 3H). m/z (MH⁺): 347.0

### Example 8: Compound #15

### 3-(1-(3-Ethoxyphenyl)-2-(methylsulfonyl)ethyl)-6-(1H-pyrazol-4-yl)quinazolin-4(3H)-one

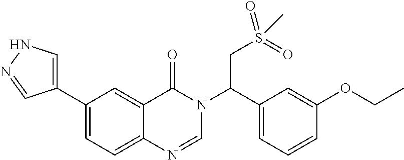 [SMILES: CCOc1cccc(C(CS(C)(=O)=O)n2cnc3ccc(-c4cn[nH]c4)cc3c2=O)c1]

Under an inert atmosphere of nitrogen, a mixture of 6-bromo-3-(1-(3-ethoxyphenyl)-2-(methylthio)ethyl)quinazolin-4(3H)-one (300 mg, 0.715 mmol, 1.00 equiv), 1-(tetrahydro-2H-pyran-2-yl)-4-(4,4,5,5-tetramethyl-1,3,2-dioxaborolan-2-yl)-1H-pyrazole (593 mg, 2.143 mmol, 3.00 equiv), K₂CO₃ (296 mg, 2.142 mmol, 3.00 equiv) and Pd(PPh₃)₄ (82.6 mg, 0.071 mmol, 0.10 equiv) in DMF (20 mL) with H₂O (2 mL) was stirred for 3 h at 100° C. EtOAc (100 mL) was added, the resulting mixture was washed with water, brine, dried over Na₂SO₄, and concentrated. The residue was applied onto a silica gel column (40 g, EA/PE: 0>>>50%>>>55%) to yield 3-(1-(3-ethoxyphenyl)-2-(methylthio)ethyl)-6-(1-(tetrahydro-2H-pyran-2-yl)-1H-pyrazol-4-yl)quinazolin-4(3H)-one as yellow solid.

To a mixture of 3-(1-(3-ethoxyphenyl)-2-(methylthio)ethyl)-6-(1-(tetrahydro-2H-pyran-2-yl)-1H-pyrazol-4-yl)quinazolin-4(3H)-one (100 mg, 0.204 mmol, 1.00 equiv) in DCM (15 mL) was added mCPBA (105 mg, 0.608 mmol, 3.00 equiv). The reaction was stirred for an overnight at room temperature. The reaction was then quenched with saturated solution of sodium bicarbonate (20 mL), extracted with DCM (3×50 mL), dried over Na₂SO₄, and concentrated. The residue was purified by Preparative TLC (EA/PE: 1:1) to yield 3-(1-(3-ethoxyphenyl)-2-(methylsulfonyl)ethyl)-6-(1-(tetrahydro-2H-pyran-2-yl)-1H-pyrazol-4-yl)quinazolin-4(3H)-one as yellow solid.

To a mixture of 3-(1-(3-ethoxyphenyl)-2-(methylsulfonyl)ethyl)-6-(1-(tetrahydro-2H-pyran-2-yl)-1H-pyrazol-4-yl)quinazolin-4(3H)-one (80 mg, 0.153 mmol, 1.00 equiv) in DCM (15 mL) was added TFA (69.8 mg, 0.612 mmol, 4.00 equiv). The reaction was stirred for 1 h, concentrated. The residue was applied onto a reverse C18 column (40 g, ACN/H₂O (0.05% TFA): 5%>>>35%>>>40%) to yield 3-(1-(3-ethoxyphenyl)-2-(methylsulfonyl)ethyl)-6-(1H-pyrazol-4-yl)quinazolin-4(3H)-one 2,2,2-trifluoroacetate as light yellow solid.

¹H NMR (400 MHz, DMSO-d6) d 8.57 (s, 1H), 8.31 (s, 1H), 8.22 (s, 2H), 8.10 (d, J=7.7 Hz, 1H), 7.86-7.95 (m, 1H), 7.51-7.75 (m, 2H), 7.31 (t, J=8.0 Hz, 1H), 7.09 (s, 1H), 7.04 (d, J=7.9 Hz, 1H), 6.92 (dd, J=8.2, 2.4 Hz, 1H), 6.45 (dd, J=10.7, 4.2 Hz, 1H), 4.61 (dd, J=14.8, 10.8 Hz, 1H), 4.32 (dd, J=14.8, 4.2 Hz, 1H), 4.03 (q, J=7.0 Hz, 2H), 3.08 (s, 3H), 1.31 (t, J=6.9 Hz, 3H); ¹⁹F NMR (376 MHz, DMSO-d6) d −74.11. m/z (MH⁺): 439.0

### Example 9: Compound #28

### 3-(3-(Oxazol-2-yl)benzyl)-6-(1H-pyrazol-4-yl)quinazolin-4(3H)-one

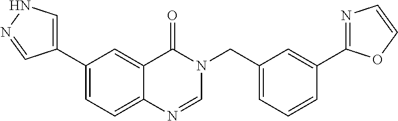 [SMILES: O=c1c2cc(-c3cn[nH]c3)ccc2ncn1Cc1cccc(-c2ncco2)c1]

Under an inert atmosphere of nitrogen, a mixture of 6-bromo-3-(3-(oxazol-2-yl)benzyl)quinazolin-4(3H)-one (170 mg, 0.445 mmol, 1.00 equiv), tert-butyl 4-(4,4,5,5-tetramethyl-1,3,2-dioxaborolan-2-yl)-1H-pyrazole-1-carboxylate (393 mg, 1.336 mmol, 3.00 equiv), Pd(PPh₃)₄ (26 mg, 0.022 mmol, 0.05 equiv), K₂CO₃ (185 mg, 1.339 mmol, 3.00 equiv) in DMF (10 mL) and H₂O (2 mL) was stirred for 4 h at 100° C. EtOAc (50 mL) was added, the resulting mixture was washed with water, brine, dried over Na₂SO₄, and concentrated. The resulting solid was re-crystalized with EtOAc to yield 3-(3-(oxazol-2-yl)benzyl)-6-(1H-pyrazol-4-yl)quinazolin-4(3H)-one as light pink solid.

¹H NMR (300 MHz, DMSO-d6) d 13.05 (brs, 1H), 8.60 (s, 1H), 8.30-8.40 (m, 2H), 8.22 (s, 1H), 8.11 (dd, J=8.5, 2.2 Hz, 2H), 8.02 (s, 1H), 7.91 (dt, J=6.8, 2.0 Hz, 1H), 7.70 (d, J=8.5 Hz, 1H), 7.49-7.61 (m, 2H), 7.37 (d, J=0.9 Hz, 1H), 5.30 (s, 2H). m/z (MH⁺): 370.1.

### Example 10: Compound #72

### N-(2,6-Difluorobenzyl)-3-fluoro-5-((4-oxo-6-(1H-pyrazol-4-yl)quinazolin-3(4H)-yl)methyl)benzamide

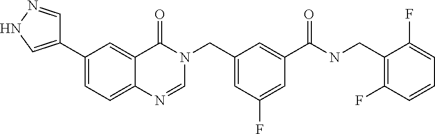 [SMILES: O=C(NCc1c(F)cccc1F)c1cc(F)cc(Cn2cnc3ccc(-c4cn[nH]c4)cc3c2=O)c1]

To A solution of tert-butyl 4-{3-[(3-{[(2,6-difluorophenyl)methyl]carbamoyl}-5-fluorophenyl)methyl]-4-oxo-3,4-dihydroquinazolin-6-yl}-1H-pyrazole-1-carboxylate (80 mg, 0.136 mmol, 1.00 equiv) in DCM (6 mL) was added TFA (775 mg, 6.797 mmol, 50.00 equiv) at room temperature. The reaction was stirred for 1 h at room temperature. The resulting mixture was quenched by the addition NaHCO₃ aq. (30 m), filtered, and solids collected to yield N-[(2,6-difluorophenyl)methyl]-3-fluoro-5-{[4-oxo-6-(1H-pyrazol-4-yl)-3,4-dihydroquinazolin-3-yl]methyl}benzamide as a white solid.

¹H NMR (DMSO-d6, 400 MHz, ppm) d 13.04 (s, 1H), 8.98 (t, J=5.2 Hz, 1H), 8.54 (s, 1H), 8.30-8.38 (m, 2H), 8.06-8.12 (m, 2H), 7.68-7.72 (m, 2H), 7.60 (dt, J=9.4, 2.3 Hz, 1H), 7.35-7.43 (m, 2H), 7.05 (t, J=7.9 Hz, 2H), 5.24 (s, 2H), 4.50 (d, J=5.1 Hz, 2H). m/z (MH⁺): 490.2.

### Example 11: Compound #1

### 3-(3-Methoxybenzyl)-4-oxo-6-(1H-pyrazol-4-yl)-3,4-dihydroquinazoline-5-carbonitrile

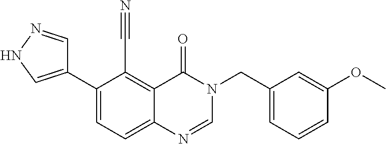 [SMILES: COc1cccc(Cn2cnc3ccc(-c4cn[nH]c4)c(C#N)c3c2=O)c1]

To a solution of 5-bromo-6-iodo-3-(3-methoxybenzyl)quinazolin-4(3H)-one (0.3 g, 0.637 mmol, 1 equiv) in DMF (10 mL) were added 1-(tetrahydro-2H-pyran-2-yl)-4-(4,4,5,5-tetramethyl-1,3,2-dioxaborolan-2-yl)-1H-pyrazole (0.354 g, 1.274 mmol, 2 equiv), H₂O (1 mL), K₂CO₃ (0.264 g, 1.910 mmol, 3 equiv), Pd(PPh₃)₄ (0.037 g, 0.032 mmol, 0.05 equiv). The resulting mixture was stirred for 5 h at 100° C. The reaction was quenched with H₂O. The resulting mixture was extracted with ethyl acetate. The organic layers were combined, dried over Na₂SO₄, filtered and concentrated. The residue was purified by silica gel chromatography (0-50% EtOAc/petroleum ether) to yield 5-bromo-3-(3-methoxybenzyl)-6-(1-(tetrahydro-2H-pyran-2-yl)-1H-pyrazol-4-yl)quinazolin-4(3H)-one as a yellow oil. LC/MS: mass calculated for C₂₄H₂₃BrN₄O₃: 495.37, measured: 495.3 [M+H]⁺.

To a solution of 5-bromo-3-(3-methoxybenzyl)-6-(1-(tetrahydro-2H-pyran-2-yl)-1H-pyrazol-4-yl)quinazolin-4(3H)-one (0.2 g, 0.404 mmol, 1 equiv) in DMF (8 mL), were added Zn(CN)₂ (0.071 g, 0.606 mmol, 1.5 equiv), Pd₂(dba)₃ (0.037 g, 0.040 mmol, 0.1 equiv.), dppf (0.045 g, 0.081 mmol, 0.2 equiv) and Zn (0.003 g, 0.040 mmol, 0.1 equiv). The resulting mixture was stirred at 80° C. overnight. The reaction was quenched with H₂O. The resulting mixture was extracted with ethyl acetate. The organic layers were combined, dried over Na₂SO₄, filtered and concentrated. The residue was purified by silica gel chromatography (0-100% EtOAc/petroleum ether) to yield 3-(3-methoxybenzyl)-4-oxo-6-(1-(tetrahydro-2H-pyran-2-yl)-1H-pyrazol-4-yl)-3,4-dihydroquinazoline-5-carbonitrile as a yellow solid.

To a solution of 3-(3-methoxybenzyl)-4-oxo-6-(1-(tetrahydro-2H-pyran-2-yl)-1H-pyrazol-4-yl)-3,4-dihydroquinazoline-5-carbonitrile (110 mg, 0.249 mmol, 1 equiv) in DCM (1 mL) was added TFA (1 mL). The resulting mixture was stirred for 2 h at 25° C. The resulting mixture was concentrated. The residue obtained was purified by C18 (0-40% MeCN/Water) to yield 3-(3-methoxybenzyl)-4-oxo-6-(1H-pyrazol-4-yl)-3,4-dihydroquinazoline-5-carbonitrile as a white solid.

LC/MS: mass calcd. for C₂₀H₁₅N₅O₂: 357.4, found: 358.1 [M+H]+; H NMR (300 MHz, DMSO-d6) d 8.68 (s, 1H), 8.28 (s, 2H), 8.15 (d, J=8.7 Hz, 1H), 7.95 (d, J=8.7 Hz, 1H), 7.28 (t, J=7.9 Hz, 1H), 6.87-6.99 (m, 3H), 5.19 (s, 2H), 3.75 (s, 3H).

### Example 12: Compound #4

### 5-Hydroxy-3-(3-methoxybenzyl)-6-(1H-pyrazol-4-yl)quinazolin-4(3H)-one

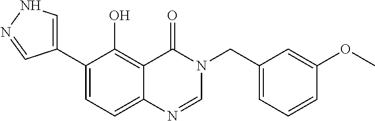 [SMILES: COc1cccc(Cn2cnc3ccc(-c4cn[nH]c4)c(O)c3c2=O)c1]

A solution of 6-bromo-5-hydroxy-3-(3-methoxybenzyl)quinazolin-4(3H)-one (100 mg, 0.277 mmol, 1.00 eq), tert-butyl 4-(4,4,5,5-tetramethyl-1,3,2-dioxaborolan-2-yl)-1H-pyrazole-1-carboxylate (162.9 mg, 0.554 mmol, 2.00 eq), potassium carbonate (114.8 mg, 0.831 mmol, 3.00 eq), Pd(PPh₃)₄ (32 mg, 0.028 mmol, 0.1 eq) in DMF/H₂O (3/0.3 mL) was stirred for 3 h at 90° C. under oil bath with an inert atmosphere of nitrogen. The reaction mixture was poured into water, extracted with ethyl acetate, washed with brine and concentrated. The residue was applied onto Prep-TLC MeOH/DCM (1:20) to yield 5-hydroxy-3-(3-methoxybenzyl)-6-(1H-pyrazol-4-yl)quinazolin-4(3H)-one as a white solid.

¹H NMR (300 MHz, DMSO-d6) d 12.96 (s, 1H), 12.48 (s, 1H), 8.48 (s, 1H), 8.24 (s, 1H), 8.09 (d, J=8.5 Hz, 1H), 7.30-7.11 (m, 2H), 6.99-6.80 (m, 3H), 5.15 (s, 2H), 3.28 (s, 2H). m/z (MH⁺): 349.2.

### Example 13: Compound #8

### 3-(3-Ethoxybenzyl)-5-fluoro-6-(1H-pyrazol-4-yl)quinazolin-4(3H)-one

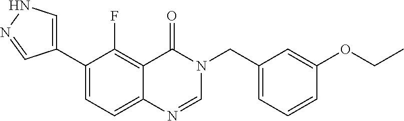 [SMILES: CCOc1cccc(Cn2cnc3ccc(-c4cn[nH]c4)c(F)c3c2=O)c1]

A solution of 6-bromo-3-(3-ethoxybenzyl)-5-fluoroquinazolin-4(3H)-one (110 mg, 0.292 mmol, 1.00 eq), tert-butyl 4-(4,4,5,5-tetramethyl-1,3,2-dioxaborolan-2-yl)-1H-pyrazole-1-carboxylate (171.560 mg, 0.583 mmol, 2.00 eq), Pd(PPh₃)₄ (33.698 mg, 0.029 mmol, 0.10 eq) and potassium carbonate (120.909 mg, 0.875 mmol, 3.00 eq) in MeOH/H₂O (5 mL/0.5 mL) was stirred for 3 h at 90° C. under oil bath with an inert atmosphere of nitrogen. The reaction mixture was quenched with water, extracted with ethyl acetate, washed with brine and concentrated. The residue was applied onto a Prep-TLC with MeOH/DCM (1:20) as solvent to yield 3-(3-ethoxybenzyl)-5-fluoro-6-(1H-pyrazol-4-yl)quinazolin-4(3H)-one as a white solid.

¹H NMR (300 MHz, DMSO-d6) d 13.17 (s, 1H), 8.50 (s, 1H), 8.25 (s, 1H), 8.15 (t, J=8.1 Hz, 1H), 8.02 (d, J=9.2 Hz, 1H), 7.48 (d, J=8.6 Hz, 1H), 7.21 (t, J=7.9 Hz, 1H), 6.92-6.76 (m, 3H), 5.09 (s, 2H), 3.96 (m, 2H), 1.55 (s, 1H), 1.25 (m, 5H). m/z (MH⁺): 365.2.

### Example 14: Compound #3

### 5-Fluoro-3-(3-methoxybenzyl)-6-(1H-pyrazol-4-yl)quinazolin-4(3H)-one

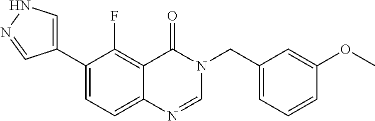 [SMILES: COc1cccc(Cn2cnc3ccc(-c4cn[nH]c4)c(F)c3c2=O)c1]

A solution of 6-bromo-5-fluoro-3-(3-methoxybenzyl)quinazolin-4(3H)-one (80 mg, 0.220 mmol, 1.00 eq), tert-butyl 4-(4,4,5,5-tetramethyl-1,3,2-dioxaborolan-2-yl)-1H-pyrazole-1-carboxylate (129.590 mg, 0.441 mmol, 2.00 eq), Pd(PPh₃)₄ (25.454 mg, 0.022 mmol, 0.10 eq) and potassium carbonate (91.330 mg, 0.661 mmol, 3.00 eq) in MeOH/H₂O (5 mL/0.5 mL) was stirred for 3 h at 90° C. with an inert atmosphere of nitrogen. The reaction mixture was quenched with water, extracted with ethyl acetate, washed with brine and concentrated. The residue was applied onto a Prep-TLC with MeOH/DCM (1:20) as solvent to yield 5-fluoro-3-(3-methoxybenzyl)-6-(1H-pyrazol-4-yl)quinazolin-4(3H)-one as a white solid.

¹H NMR (300 MHz, DMSO-d6) d 13.16 (s, 1H), 8.50 (s, 1H), 8.26 (s, 1H), 8.15 (t, J=8.1 Hz, 1H), 8.00 (s, 1H), 7.48 (d, J=8.6 Hz, 1H), 7.23 (t, J=7.8 Hz, 1H), 6.96-6.79 (m, 3H), 5.10 (s, 2H), 3.70 (s, 3H), 2.45 (s, 2H). m/z (MH⁺): 351.15.

### Example 15: Compound #17

### 3-(1-(3-(2-(Methylsulfonyl)ethoxy)phenyl)ethyl)-6-(1H-pyrazol-4-yl)quinazolin-4(3H)-one

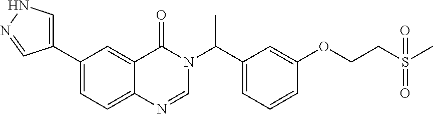 [SMILES: CC(c1cccc(OCCS(C)(=O)=O)c1)n1cnc2ccc(-c3cn[nH]c3)cc2c1=O]

Into a 25-mL round-bottom flask purged and maintained with an inert atmosphere of nitrogen, was placed a solution of 3-(1-(3-hydroxyphenyl)ethyl)-6-(1-(tetrahydro-2H-pyran-2-yl)-1H-pyrazol-4-yl)quinazolin-4(3H)-one (140 mg, 0.336 mmol, 1.00 eq), 2-chloroethyl methyl sulfide (185 mg, 1.673 mmol, 5.00 eq) and K₂CO₃ (139 mg, 1.006 mmol, 3.00 eq) in DMF (6 mL). The resulting mixture was stirred for 16 h at 100° C. The reaction mixture was quenched with water, extracted with ethyl acetate, washed with brine and concentrated. The residue was applied onto a silica gel with EA/PE (60%) to yield 3-(1-(3-(2-(methylthio)ethoxy)phenyl)ethyl)-6-(1-(tetrahydro-2H-pyran-2-yl)-1H-pyrazol-4-yl)quinazolin-4(3H)-one as a light brown oil.

Into a 25-mL round-bottom flask purged and maintained with an inert atmosphere of nitrogen, was placed a solution of 3-(1-(3-(2-(methylthio)ethoxy)phenyl)ethyl)-6-(1-(tetrahydro-2H-pyran-2-yl)-1H-pyrazol-4-yl)quinazolin-4(3H)-one (140 mg, 0.285 mmol, 1.00 eq) in DCM (6 mL). To the resulting mixture was then added 3-chloroperoxybenzoic acid (123 mg, 0.713 mmol, 2.50 eq). The resulting mixture was stirred for 4 h at room temperature. The reaction mixture was quenched with water, extracted with ethyl acetate, washed with brine and concentrated. The residue was applied onto a silica gel with EA/PE (60%) to yield 3-(1-(3-(2-(methylsulfonyl)ethoxy)phenyl)ethyl)-6-(1-(tetrahydro-2H-pyran-2-yl)-1H-pyrazol-4-yl)quinazolin-4(3H)-one as a light brown oil.

Trifluoroacetic acid (1 mL) was added to a solution of 3-(1-(3-(2-(methylsulfonyl)ethoxy)phenyl)ethyl)-6-(1-(tetrahydro-2H-pyran-2-yl)-1H-pyrazol-4-yl)quinazolin-4(3H)-one (130 mg, 0.249 mmol, 1.00 eq) in DCM (5 mL). The resulting mixture was stirred for 4 h at room temperature. The reaction mixture was quenched with NaHCO₃/H₂O, extracted with EA/MeOH (90%), washed with brine and concentrated. The residue was applied onto Prep-TLC with MeOH/DCM (1:10) to yield 3-(1-(3-(2-(Methylsulfonyl)ethoxy)phenyl)ethyl)-6-(1H-pyrazol-4-yl)quinazolin-4(3H)-one as a white solid.

¹H NMR (400 MHz, DMSO-d6) d 13.05 (s, 1H), 8.37 (s, 2H), 8.30 (d, J=2.1 Hz, 1H), 8.11-8.01 (m, 2H), 7.65 (d, J=8.5 Hz, 1H), 7.30 (t, J=8.0 Hz, 1H), 7.07-6.89 (m, 3H), 6.08 (q, J=7.2 Hz, 1H), 4.38-4.28 (m, 2H), 3.59 (t, J=5.6 Hz, 2H), 3.05 (s, 3H), 1.83 (d, J=7.2 Hz, 3H). m/z (MH⁺): 439.0

### Example 16: Compound #22

### 3-((1,1-Dioxidotetrahydro-2H-thiopyran-4-yl)(3-methoxyphenyl)methyl)-6-(1H-pyrazol-4-yl)quinazolin-4(3H)-one

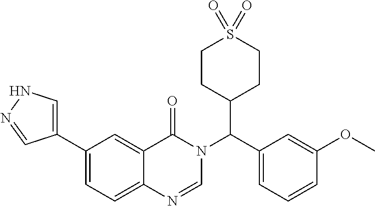 [SMILES: COc1cccc(C(C2CCS(=O)(=O)CC2)n2cnc3ccc(-c4cn[nH]c4)cc3c2=O)c1]

Into a 25-mL round-bottom flask purged and maintained with an inert atmosphere of nitrogen, was placed a solution of 6-bromo-3-((1,1-dioxidotetrahydro-2H-thiopyran-4-yl)(3-methoxyphenyl)methyl)quinazolin-4(3H)-one (100 mg, 0.209 mmol, 1.00 eq), tert-butyl 4-(4,4,5,5-tetramethyl-1,3,2-dioxaborolan-2-yl)-1H-pyrazole-1-carboxylate (123 mg, 0.418 mmol, 2.00 eq), potassium carbonate (87 mg, 0.629 mmol, 3.00 eq) and tetrakis(triphenylphosphine) Palladium(0) (24 mg, 0.021 mmol, 0.10 eq) in DMF/H₂O (5/0.5 mL). The resulting mixture was stirred for 16 h at 100° C. The reaction mixture was poured into water, extracted with EA/MeOH (90%), washed with brine and concentrated. The residue was applied onto Prep-TLC with MeOH/DCM (1:20) to yield 3-((1,1-dioxidotetrahydro-2H-thiopyran-4-yl)(3-methoxyphenyl)methyl)-6-(1H-pyrazol-4-yl)quinazolin-4(3H)-one as an off-white solid.

¹H NMR (300 MHz, DMSO-d6) d 13.02 (s, 1H), 8.49 (s, 1H), 8.34 (s, 1H), 8.26 (d, J=2.1 Hz, 1H), 8.04 (dd, J=8.7, 2.4 Hz, 2H), 7.60 (d, J=8.5 Hz, 1H), 7.29 (t, J=8.1 Hz, 1H), 7.15 (d, J=7.0 Hz, 2H), 6.88 (d, J=7.8 Hz, 1H), 5.64 (d, J=12.1 Hz, 1H), 3.72 (s, 3H), 3.08 (d, J=23.8 Hz, 5H), 1.80 (s, 2H), 1.70 (s, 2H). m/z (MH⁺): 465.0.

### Example 17: Compound #25

### 2-Methyl-3-(3-((1-methylazetidin-3-yl)oxy)benzyl)-6-(1H-pyrazol-4-yl)quinazolin-4(3H)-one

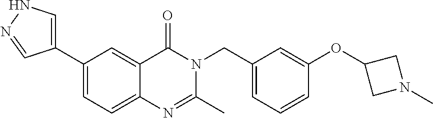 [SMILES: Cc1nc2ccc(-c3cn[nH]c3)cc2c(=O)n1Cc1cccc(OC2CN(C)C2)c1]

Into a 100-mL round-bottom flask purged and maintained with an inert atmosphere of nitrogen, was placed a solution of 6-bromo-3-(3-(tert-butyldimethylsilyloxy)benzyl)-2-methylquinazolin-4(3H)-one (750 mg, 1.632 mmol, 1.00 eq), 1-(tetrahydro-2H-pyran-2-yl)-4-(4,4,5,5-tetramethyl-1,3,2-dioxaborolan-2-yl)-1H-pyrazole (544 mg, 1.956 mmol, 1.20 eq), potassium carbonate (675 mg, 4.884 mmol, 3.00 eq) and tetrakis(triphenylphosphine) palladium(0) (189 mg, 0.164 mmol, 0.10 eq) in DME/H₂O (15/1.5 mL). The resulting mixture was stirred for 16 h at 85° C. The reaction mixture was poured into water, extracted with ethyl acetate, washed with brine and concentrated. The residue was applied onto a silica gel column with EA/PE (35%) to yield 3-(3-(tert-butyldimethylsilyloxy)benzyl)-2-methyl-6-(1-(tetrahydro-2H-pyran-2-yl)-1H-pyrazol-4-yl)quinazolin-4(3H)-one as a light brown oil.

Into a 100-mL round-bottom flask purged and maintained with an inert atmosphere of nitrogen, was placed a solution of 3-(3-(tert-butyldimethylsilyloxy)benzyl)-2-methyl-6-(1-(tetrahydro-2H-pyran-2-yl)-1H-pyrazol-4-yl)quinazolin-4(3H)-one (300 mg, 0.565 mmol, 1.00 eq) in THE (8 mL). To the resulting mixture was then added tetrabutylammonium fluoride (1.13 mL, 1.13 mmol, 2.00 eq, 1M in THF). The resulting mixture was stirred for 1.5 h at room temperature. The reaction mixture was poured into water, extracted with ethyl acetate, washed with brine and concentrated. The residue was applied onto a silica gel column with EA/PE (50%) to yield 3-(3-hydroxybenzyl)-2-methyl-6-(1-(tetrahydro-2H-pyran-2-yl)-1H-pyrazol-4-yl)quinazolin-4(3H)-one as a yellow solid.

Into a 50-mL round-bottom flask purged and maintained with an inert atmosphere of nitrogen, was placed a solution of 3-(3-hydroxybenzyl)-2-methyl-6-(1-(tetrahydro-2H-pyran-2-yl)-1H-pyrazol-4-yl)quinazolin-4(3H)-one (160 mg, 0.384 mmol, 1.00 eq), tert-butyl 3-(methylsulfonyloxy)azetidine-1-carboxylate (193 mg, 0.768 mmol, 2.00 eq) and cesium carbonate (376 mg, 1.154 mmol, 3.00 eq) in DMF (8 mL). The resulting mixture was stirred for 16 h at 100° C. The reaction mixture was poured into water, extracted with ethyl acetate, washed with brine and concentrated. The residue was applied onto a silica gel column with EA/PE (85%) to yield tert-butyl 3-(3-((2-methyl-4-oxo-6-(1-(tetrahydro-2H-pyran-2-yl)-1H-pyrazol-4-yl)quinazolin-3(4H)-yl)methyl)phenoxy)azetidine-1-carboxylate as a brown oil.

Into a 25-mL round-bottom flask was placed a solution of tert-butyl 3-(3-((2-methyl-4-oxo-6-(1-(tetrahydro-2H-pyran-2-yl)-1H-pyrazol-4-yl)quinazolin-3(4H)-yl)methyl)phenoxy)azetidine-1-carboxylate (150 mg, 0.262 mmol, 1.00 eq) in TFA/DCM (2/4 mL). The resulting mixture was stirred for 8 h at room temperature. The reaction mixture was added into water dropwise, extracted with DCM/MeOH (90%) and concentrated to yield 3-(3-(azetidin-3-yloxy)benzyl)-2-methyl-6-(1H-pyrazol-4-yl)quinazolin-4(3H)-one as a brown oil.

Into a 10-mL round-bottom flask purged and maintained with an inert atmosphere of nitrogen, was placed a solution of 3-(3-(azetidin-3-yloxy)benzyl)-2-methyl-6-(1H-pyrazol-4-yl)quinazolin-4(3H)-one (100 mg, 0.258 mmol, 1.00 eq) in MeOH (6 mL). To the resulting mixture was then added formaldehyde (2 mL, 37% in H₂) dropwise. The resulting mixture was stirred for 30 min at room temperature. Sodium cyanoborohydride (81 mg, 1.289 mmol, 5.00 eq) was then added. The resulting mixture was stirred for 8 h at room temperature.

The reaction was poured into water, extracted with EA/MeOH (90%), washed with brine and concentrated. The residue was applied onto a C18 reverse column with CH₃CN/H₂O (0.05% NH₄HCO₃) (80%) to yield 2-Methyl-3-(3-((1-methylazetidin-3-yl)oxy)benzyl)-6-(1H-pyrazol-4-yl)quinazolin-4(3H)-one as an off-white solid.

¹H NMR (400 MHz, DMSO-d6) d 13.07 (s, 1H), 8.51-8.23 (m, 2H), 8.10 (dd, J=8.5, 2.2 Hz, 2H), 7.63 (d, J=8.4 Hz, 1H), 7.27 (t, J=7.9 Hz, 1H), 6.83-6.71 (m, 2H), 6.66 (d, J=2.2 Hz, 1H), 5.46-5.27 (m, 2H), 4.73 (t, J=5.6 Hz, 1H), 3.75 (dd, J=7.9, 5.9 Hz, 2H), 3.01 (t, J=6.4 Hz, 2H), 2.48 (s, 3H), 2.31 (s, 3H). m/z (MH⁺): 402.1

### Example 18: Compound #69

### 3-benzyl-7-fluoro-6-(1H-pyrazol-4-yl)quinazolin-4(3H)-one

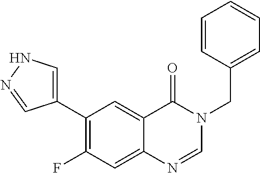 [SMILES: O=c1c2cc(-c3cn[nH]c3)c(F)cc2ncn1Cc1ccccc1]

A mixture of 3-benzyl-6-bromo-7-fluoroquinazolin-4(3H)-one (119.4 mg, 0.62 mmol), tert-butyl 4-(4,4,5,5-tetramethyl-1,3,2-dioxaborolan-2-yl)-1H-pyrazole-1-carboxylate (82 mg, 0.25 mmol), 2M aqueous potassium carbonate (0.25 ml, 0.49 mmol) in 1,4-dioxine (2 ml) was degassed with nitrogen, then treated with Pd(PPh₃)₄ (14.2 mg, 0.012 mmol). The reaction mixture was heated to 120° C. for 50 mins under microwave irradiation. The resulting mixture was cooled to room temperature, poured into aqueous NH₄Cl solution, extracted with EtOAc three times. The combined organic layer was dried, concentrated, and the residue was purified by flash column chromatography on silica gel (12 g, EtOAc/heptane: 0>>>40%>>>90%) to yield 3-benzyl-7-fluoro-6-(1H-pyrazol-4-yl)quinazolin-4(3H)-one as a white solid.

¹H NMR (400 MHz, CHLOROFORM-d) δ 8.55 (d, J=8.08 Hz, 1H), 8.10 (d, J=1.52 Hz, 3H), 7.42-7.73 (m, 2H), 7.32-7.40 (m, 5H), 5.21 (s, 2H). m/z (MH⁺): 321.10

### Example 19: Compound #67

### 3-(3-Fluoro-4-methoxybenzyl)-6-(1H-pyrazol-4-yl)quinazolin-4(3H)-one

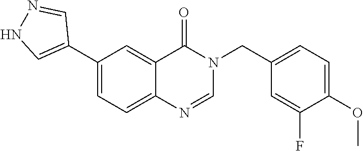 [SMILES: COc1ccc(Cn2cnc3ccc(-c4cn[nH]c4)cc3c2=O)cc1F]

A mixture of 6-bromoquinazolin-4-ol, 4-(bromomethyl)-1-fluoro-2-methoxybenzene and K₂CO₃ in acetonitrile (4 ml) was kept stirring at 60° C. for 3 h. The solid was filtered off and the filtrate was concentrated. The residue was purified by flash column chromatography on silica gel (12 g, EtOAc/heptane) to yield 6-bromo-3-(3-fluoro-4-methoxybenzyl)quinazolin-4(3H)-one as a white solid. ¹H NMR (CHLOROFORM-d) Shift: 8.46 (d, J=2.5 Hz, 1H), 8.10 (s, 1H), 7.84 (dd, J=8.8, 2.3 Hz, 1H), 7.59 (d, J=8.6 Hz, 1H), 6.96-7.11 (m, 2H), 6.80-6.93 (m, 1H), 5.14 (s, 2H), 3.88 (s, 3H). m/z (MH⁺): 363.0.

A mixture of 4-(4,4,5,5-tetramethyl-1,3,2-dioxaborolan-2-yl)-1H-pyrazole (75.6 mg, 0.39 mmol), 6-bromo-3-(3-fluoro-4-methoxybenzyl)quinazolin-4(3H)-one (56.6 mg, 0.16 mmol), 2M aqueous potassium carbonate (0.16 ml, 0.32 mmol) in 1,4-dioxine (2 ml) was degassed with nitrogen, then treated with Pd(PPh₃)₄ (9.0 mg, 0.008 mmol). The reaction mixture was heated to 120° C. for 50 mins under microwave irradiation. The resulting mixture was cooled to room temperature, poured into aqueous NH₄Cl solution, extracted with EtOAc three times. The combined organic layer was dried, concentrated, and the residue was purified by flash column chromatography on silica gel (12 g, EtOAc/heptane) to yield 3-(3-Fluoro-4-methoxybenzyl)-6-(1H-pyrazol-4-yl)quinazolin-4(3H)-one as a white solid.

¹H NMR (400 MHz, METHANOL-d₄) δ 8.37-8.43 (m, 2H), 8.12-8.25 (m, 1H), 8.08 (dd, J=2.02, 8.59 Hz, 2H), 7.69 (d, J=8.08 Hz, 1H), 7.14-7.27 (m, 2H), 7.01-7.12 (m, 1H), 5.18 (s, 2H), 3.84 (s, 3H). m/z (MH⁺): 351.0

### Example 20: Compound #52

### 6-(2-Aminopyrimidin-5-yl)-3-(3-methoxybenzyl)quinazolin-4(3H)-one

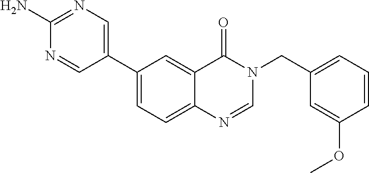 [SMILES: COc1cccc(Cn2cnc3ccc(-c4cnc(N)nc4)cc3c2=O)c1]

A mixture of 5-(4,4,5,5-tetramethyl-1,3,2-dioxaborolan-2-yl)pyrimidin-2-amine (80.1 mg, 0.36 mmol), 6-bromo-3-(3-methoxybenzyl)quinazolin-4(3H)-one (50 mg, 0.15 mmol), 2M aqueous potassium carbonate (0.15 ml, 0.29 mmol) in 1,4-dioxine (2 ml) was degassed with nitrogen, then treated with Pd(PPh₃)₄ (8.4 mg, 0.007 mmol). The reaction mixture was heated to 120° C. to 65 mins under microwave irradiation. The resulting mixture was concentrated, diluted with water and the precipitate was filtered off, washed with EtOAc and MeOH to yield 6-(2-aminopyrimidin-5-yl)-3-(3-methoxybenzyl)quinazolin-4(3H)-one as a white solid.

¹H NMR (400 MHz, DMSO-d₆) δ 8.69 (s, 2H), 8.57 (s, 1H), 8.28-8.33 (m, 1H), 8.07-8.16 (m, 1H), 7.71-7.79 (m, 1H), 7.27 (t, J=7.83 Hz, 1H), 6.85-6.99 (m, 5H), 5.19 (s, 2H), 3.73 (s, 3H). m/z (MH⁺): 360.1

The following compounds were similarly prepared, following the procedure described in the Examples and Schemes herein, and selecting and substituting suitable reactants, as would be readily recognized by those skilled in the art.

### Example 21: Compound #83

### 3-Benzyl-6-pyrimidin-5-yl-quinazolin-4-one

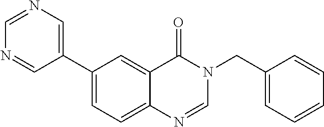 [SMILES: O=c1c2cc(-c3cncnc3)ccc2ncn1Cc1ccccc1]

¹H NMR (400 MHz, CHLOROFORM-d) δ 9.26 (s, 1H), 9.06 (s, 2H), 8.57 (d, J=2.02 Hz, 1H), 8.19 (s, 1H), 7.97 (d, J=2.02 Hz, 1H), 7.87 (d, J=8.59 Hz, 1H), 7.30-7.42 (m, 5H), 5.25 (s, 2H). m/z (MH⁺): 315.1

### Example 22: Compound #82

### 3-Benzyl-6-(6-fluoro-3-pyridyl)quinazolin-4-one

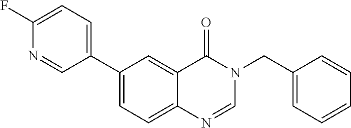 [SMILES: O=c1c2cc(-c3ccc(F)nc3)ccc2ncn1Cc1ccccc1]

¹H NMR (400 MHz, CHLOROFORM-d) δ 8.50 (dd, J=2.53, 8.08 Hz, 2H), 8.17 (s, 1H), 8.07 (br d, J=2.53 Hz, 1H), 7.93 (dd, J=2.02, 8.59 Hz, 1H), 7.81 (d, J=8.08 Hz, 1H), 7.30-7.41 (m, 5H), 7.05 (dd, J=3.03, 8.59 Hz, 1H), 5.23 (s, 2H). m/z (MH⁺): 332.1

### Example 23: Compound #79

### 3-Benzyl-6-(1H-pyrazol-4-yl)quinazolin-4-one

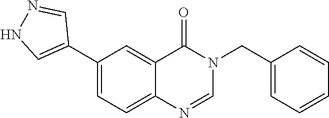 [SMILES: O=c1c2cc(-c3cn[nH]c3)ccc2ncn1Cc1ccccc1]

¹H NMR (400 MHz, METHANOL-d₄) δ 8.58 (s, 1H), 8.40 (d, J=2.02 Hz, 1H), 8.16 (br s, 2H), 8.08 (dd, J=2.27, 8.34 Hz, 1H), 7.69 (d, J=8.59 Hz, 1H), 7.29-7.46 (m, 5H), 5.27 (s, 2H). m/z (MH⁺): 303.0

### Example 24: Compound #81

### 3-Benzyl-6-(4-pyridyl)quinazolin-4-one

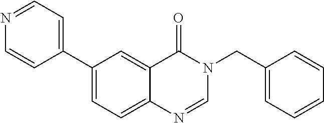 [SMILES: O=c1c2cc(-c3ccncc3)ccc2ncn1Cc1ccccc1]

¹H NMR (400 MHz, CHLOROFORM-d) δ 8.69-8.74 (m, 2H), 8.60-8.65 (m, 1H), 8.16 (s, 1H), 8.04 (dd, J=2.02, 8.59 Hz, 1H), 7.83 (d, J=8.59 Hz, 1H), 7.59-7.64 (m, 2H), 7.32-7.40 (m, 5H), 5.24 (s, 2H). m/z (MH⁺): 314.1

### Example 25: Compound #80

### 3-Benzyl-6-(1H-pyrazol-5-yl)quinazolin-4-one

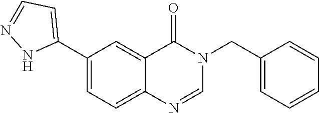 [SMILES: O=c1c2cc(-c3ccn[nH]3)ccc2ncn1Cc1ccccc1]

¹H NMR (400 MHz, METHANOL-d₄) δ 8.59-8.65 (m, 1H), 8.51-8.57 (m, 1H), 8.23-8.30 (m, 1H), 7.74 (br s, 2H), 7.29-7.47 (m, 5H), 6.82 (d, J=1.52 Hz, 1H), 5.24-5.30 (m, 2H). m/z (MH⁺): 303.0

### Example 26: Compound #78

### 3-[(2,6-Difluorophenyl)methyl]-6-(1H-pyrazol-4-yl)quinazolin-4-one

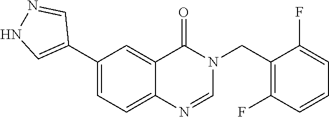 [SMILES: O=c1c2cc(-c3cn[nH]c3)ccc2ncn1Cc1c(F)cccc1F]

¹H NMR (400 MHz, METHANOL-d₄) δ 8.39-8.44 (m, 1H), 8.27-8.31 (m, 1H), 8.09-8.21 (m, 1H), 8.02-8.07 (m, 1H), 7.96-8.01 (m, 1H), 7.63-7.74 (m, 1H), 7.30-7.45 (m, 1H), 7.00 (s, 2H), 5.31 (s, 2H). m/z (MH⁺): 339.0

### Example 27: Compound #77

### 3-[(2,6-Difluorophenyl)methyl]-6-[3-(trifluoromethyl)-1H-pyrazol-4-yl]quinazolin-4-one

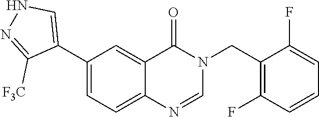 [SMILES: O=c1c2cc(-c3c[nH]nc3C(F)(F)F)ccc2ncn1Cc1c(F)cccc1F]

¹H NMR (400 MHz, METHANOL-d₄) δ 8.48 (s, 1H), 8.24 (d, J=2.02 Hz, 1H), 8.05 (s, 1H), 7.90 (dd, J=2.02, 8.59 Hz, 1H), 7.74 (d, J=8.08 Hz, 1H), 7.35-7.45 (m, 1H), 6.97-7.08 (m, 2H), 5.28-5.37 (m, 2H). m/z (MH⁺): 407.10

### Example 28: Compound C3

### N-[(2,6-Difluorophenyl)methyl]-4-[[4-oxo-6-(1H-pyrazol-4-yl)quinazolin-3-yl]methyl]benzamide

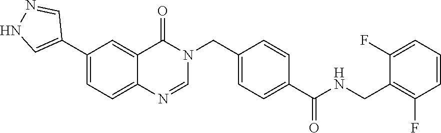 [SMILES: O=C(NCc1c(F)cccc1F)c1ccc(Cn2cnc3ccc(-c4cn[nH]c4)cc3c2=O)cc1]

¹H NMR (400 MHz, METHANOL-d₄) δ 8.40 (s, 1H), 8.37 (d, J=2.02 Hz, 1H), 8.07 (dd, J=2.27, 8.34 Hz, 3H), 7.78 (d, J=8.08 Hz, 2H), 7.69 (d, J=8.08 Hz, 1H), 7.45 (d, J=8.08 Hz, 2H), 7.27-7.37 (m, 1H), 6.96 (t, J=7.83 Hz, 2H), 5.30 (s, 2H), 4.59-4.65 (m, 2H). m/z (MH⁺): 472.05

### Example 29: Compound C2

### N-[(2,4-Difluorophenyl)methyl]-4-[[4-oxo-6-(1H-pyrazol-4-yl)quinazolin-3-yl]methyl]benzamide

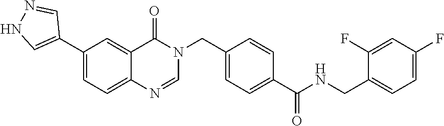 [SMILES: O=C(NCc1ccc(F)cc1F)c1ccc(Cn2cnc3ccc(-c4cn[nH]c4)cc3c2=O)cc1]

A mixture of 4-((6-bromo-4-oxoquinazolin-3(4H)-yl)methyl)benzoic acid (70.2 mg, 0.2 mmol), EDCl (49 mg, 0.25 mmol), HOBt (26 mg, 0.2 mmol), (2,4-difluorophenyl)methanamine (28 mg, 0.2 mmol) and DIEA (0.9 ml) in THE (6 mL) was stirred at room temperature for 16 h. The resulting mixture was washed with 2N HCl twice and the organic layer was separated, dried and filtered. The filtrate was concentrated under reduced pressure and the resulting residue was washed with MeOH three times, then dried in vacuo to yield 4-((6-bromo-4-oxoquinazolin-3(4H)-yl)methyl)-N-(2,4-difluorobenzyl)benzamide as a white solid.

A mixture of tert-butyl 4-(4,4,5,5-tetramethyl-1,3,2-dioxaborolan-2-yl)-1H-pyrazole-1-carboxylate (34.3 mg, 0.18 mmol), 4-((6-bromo-4-oxoquinazolin-3(4H)-yl)methyl)-N-(2,4-difluorobenzyl)benzamide (34.2 mg, 0.07 mmol), 2M aqueous potassium carbonate (0.08 ml, 0.14 mmol) in 1,4-dioxine was degassed with nitrogen, then treated with Pd(PPh₃)₄ (4.1 mg, 0.004 mmol). The reaction mixture was heated to 120° C. for 50 mins under microwave irradiation. The resulting mixture was cooled to room temperature, poured into aqueous NH₄Cl solution, extracted with EtOAc three times. The combined organic layer was dried, concentrated, and the residue was purified by flash column chromatography on silica gel (12 g, EtOAc/heptane: 0>>>40%>>>90%) to yield N-[(2,4-difluorophenyl)methyl]-4-[[4-oxo-6-(1H-pyrazol-4-yl)quinazolin-3-yl]methyl]benzamide as a white solid.

¹H NMR (MeOD) Shift: 8.35-8.45 (m, 2H), 8.16 (br. s., 1H), 8.08 (dd, J=8.3, 2.3 Hz, 2H), 7.83 (d, J=8.6 Hz, 2H), 7.70 (d, J=8.6 Hz, 1H), 7.48 (d, J=8.6 Hz, 2H), 7.34-7.45 (m, 1H), 6.84-7.02 (m, 2H), 5.32 (s, 2H), 4.56 (s, 2H). m/z (MH)⁺: 472.05.

The following compounds were similarly prepared, following the procedure described in the Examples and Schemes herein, and selecting and substituting suitable reactants, as would be readily recognized by those skilled in the art.

### Example 30: Compound #68

### 3-Benzyl-8-fluoro-6-(1H-pyrazol-4-yl)quinazolin-4-one

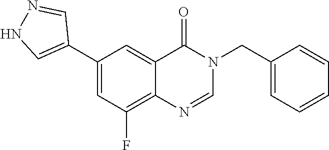 [SMILES: O=c1c2cc(-c3cn[nH]c3)cc(F)c2ncn1Cc1ccccc1]

¹H NMR (400 MHz, CHLOROFORM-d) δ 8.23 (s, 1H), 8.13 (s, 1H), 7.97 (s, 2H), 7.64 (dd, J=1.77, 10.86 Hz, 1H), 7.31-7.42 (m, 5H), 5.23 (s, 2H). m/z (MH⁺): 321.0

### Example 31: Compound #66

### 6-(1H-Pyrazol-4-yl)-3-[[3-(trifluoromethoxy)phenyl]methyl]quinazolin-4-one

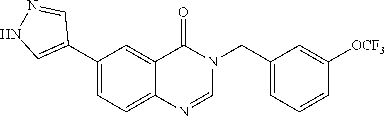 [SMILES: O=c1c2cc(-c3cn[nH]c3)ccc2ncn1Cc1cccc(OC(F)(F)F)c1]

¹H NMR (400 MHz, METHANOL-d₄) δ 8.41-8.48 (m, 2H), 7.97-8.25 (m, 3H), 7.72 (d, J=8.59 Hz, 1H), 7.44-7.50 (m, 1H), 7.35-7.42 (m, 2H), 7.19-7.28 (m, 1H), 5.30 (s, 2H). m/z (MH⁺): 387.15

### Example 32: Compound #64

### 3-[[3-(Difluoromethoxy)phenyl]methyl]-6-(1H-pyrazol-4-yl)quinazolin-4-one

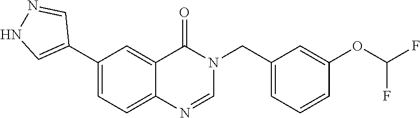 [SMILES: O=c1c2cc(-c3cn[nH]c3)ccc2ncn1Cc1cccc(OC(F)F)c1]

¹H NMR (400 MHz, METHANOL-d₄) δ 8.36-8.44 (m, 2H), 8.00-8.24 (m, 3H), 7.65-7.79 (m, 1H), 7.39 (s, 1H), 7.22 (s, 2H), 7.05-7.14 (m, 1H), 6.82 (s, 1H), 5.27 (s, 2H). m/z (MH⁺): 369.15

### Example 33: Compound #65

### 3-[[3-(4-Fluorophenoxy)phenyl]methyl]-6-(1H-pyrazol-4-yl)quinazolin-4-one

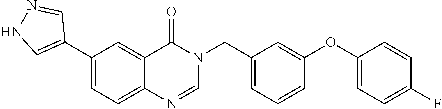 [SMILES: O=c1c2cc(-c3cn[nH]c3)ccc2ncn1Cc1cccc(Oc2ccc(F)cc2)c1]

¹H NMR (400 MHz, METHANOL-d₄) δ 8.34-8.45 (m, 2H), 7.97-8.22 (m, 3H), 7.67-7.74 (m, 1H), 7.28-7.37 (m, 1H), 7.02-7.16 (m, 3H), 6.95-7.01 (m, 3H), 6.88 (dd, J=2.53, 8.08 Hz, 1H), 5.19-5.31 (m, 2H). m/z (MH⁺): 413.10

### Example 34: Compound #63

### 3-[(3-Benzyloxyphenyl)methyl]-6-(1H-pyrazol-4-yl)quinazolin-4-one

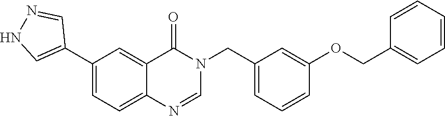 [SMILES: O=c1c2cc(-c3cn[nH]c3)ccc2ncn1Cc1cccc(OCc2ccccc2)c1]

¹H NMR (400 MHz, CHLOROFORM-d) δ 10.75-11.29 (m, 1H), 8.41-8.51 (m, 1H), 8.06 (s, 1H), 7.99 (s, 2H), 7.92 (dd, J=2.02, 8.59 Hz, 1H), 7.73 (d, J=8.59 Hz, 1H), 7.27-7.41 (m, 6H), 6.89-6.98 (m, 3H), 5.19 (s, 2H), 5.03 (s, 2H). m/z (MH⁺): 409.20

### Example 35: Compound #62

### 3-[(4-Fluoro-3-methoxy-phenyl)methyl]-6-(1H-pyrazol-4-yl)quinazolin-4-one

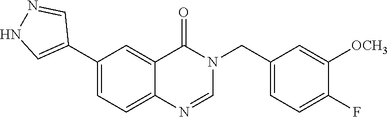 [SMILES: COc1cc(Cn2cnc3ccc(-c4cn[nH]c4)cc3c2=O)ccc1F]

¹H NMR (400 MHz, MeOH) δ 8.37-8.47 (m, 2H), 7.96-8.24 (m, 3H), 7.71 (d, J=8.08 Hz, 1H), 7.20-7.26 (m, 1H), 7.03-7.10 (m, 1H), 6.96 (tdd, J=2.21, 4.11, 6.13 Hz, 1H), 5.23 (s, 2H), 3.86 (s, 3H). m/z (MH⁺): 351.15

### Example 36: Compound #61

### N-[(4-Fluorophenyl)methyl]-3-[1-[4-oxo-6-(1H-pyrazol-4-yl)quinazolin-3-yl]ethyl]benzamide

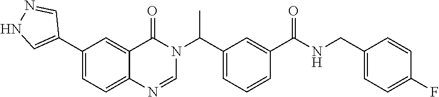 [SMILES: CC(c1cccc(C(=O)NCc2ccc(F)cc2)c1)n1cnc2ccc(-c3cn[nH]c3)cc2c1=O]

¹H NMR (300 MHz, DMSO-d6) d 13.06 (s, 1H), 9.09 (t, J=6.0 Hz, 1H), 8.38 (d, J=3.6 Hz, 2H), 8.31 (d, J=2.1 Hz, 1H), 8.01-8.14 (m, 2H), 7.92 (s, 1H), 7.83 (d, J=7.7 Hz, 1H), 7.66 (d, J=8.5 Hz, 1H), 7.58 (d, J=7.7 Hz, 1H), 7.48 (t, J=7.7 Hz, 1H), 7.29-7.39 (m, 2H), 7.07-7.18 (m, 2H), 6.13 (t, J=7.2 Hz, 1H), 4.44 (d, J=6.0 Hz, 2H), 1.89 (d, J=7.2 Hz, 3H). m/z (MH⁺): 468.3

### Example 37: Compound #60

### N-(4-Fluorophenyl)-3-[[4-oxo-6-(1H-pyrazol-4-yl)quinazolin-3-yl]methyl]benzamide

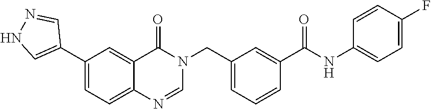 [SMILES: O=C(Nc1ccc(F)cc1)c1cccc(Cn2cnc3ccc(-c4cn[nH]c4)cc3c2=O)c1]

¹H NMR (300 MHz, DMSO-d6) d 13.02 (s, 1H), 10.29 (s, 1H), 8.54 (s, 1H), 8.34 (s, 1H), 8.27 (d, J=2.1 Hz, 1H), 7.99-8.11 (m, 2H), 7.91 (t, J=1.7 Hz, 1H), 7.84 (dt, J=7.7, 1.5 Hz, 1H), 7.62-7.77 (m, 3H), 7.56 (dt, J=7.7, 1.5 Hz, 1H), 7.47 (t, J=7.6 Hz, 1H), 7.09-7.21 (m, 2H), 5.26 (s, 2H). m/z (MH⁺): 440.0.

### Example 38: Compound C1

### N-[(4-Fluorophenyl)methyl]-3-[4-oxo-6-(1H-pyrazol-4-yl)quinazolin-3-yl]benzamide

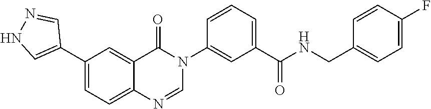 [SMILES: O=C(NCc1ccc(F)cc1)c1cccc(-n2cnc3ccc(-c4cn[nH]c4)cc3c2=O)c1]

Into a 100-mL round-bottom flask purged and maintained with an inert atmosphere of nitrogen, was placed a solution of methyl 3-(1-(2-amino-5-bromobenzamido)ethyl)benzoate (1.0 g, 4.629 mmol, 1.00 equiv), methyl orthoformate (2.5 g, 23.558 mmol, 5.00 equiv), acetic acid (0.5 mL) in toluene (25 mL). The resulting mixture was stirred for 3 h at 120° C. The reaction mixture was then cooled to 40° C. and methyl 3-aminobenzoate (700 mg, 4.631 mmol, 1.00 equiv) was added. The mixture was stirred for 16 h at 120° C. The reaction mixture was quenched with brine, extracted with ethyl acetate, washed with brine and concentrated. The residue was applied onto a silica gel with EA/PE (40%) to yield methyl 3-(6-bromo-4-oxoquinazolin-3(4H)-yl)benzoate as an off-white solid.

Into a 50-mL round-bottom flask, was placed a solution of methyl 3-(6-bromo-4-oxoquinazolin-3(4H)-yl)benzoate (200 mg, 0.557 mmol, 1.00 equiv) in THE (1.5 mL). To the resulting mixture was then added\methanol (0.7 mL). A solution of lithium hydroxide hydrate (70.2 mg, 1.673 mmol, 3.00 equiv) in H₂O (0.7 mL) was added. The resulting mixture was stirred for 6 h at room temperature. THE was removed through rotavapor and the mixture was adjusted to pH 7 with 1 M HCl (aq) and then concentrated to yield 3-(6-bromo-4-oxoquinazolin-3(4H)-yl)benzoic acid as a off-white solid.

Into a 25-mL round-bottom flask purged and maintained with an inert atmosphere of nitrogen, was placed a solution of 3-(6-bromo-4-oxoquinazolin-3(4H)-yl)benzoic acid (165 mg, 0.478 mmol, 1.00 equiv), 4-fluorobenzylamine (78 mg, 0.623 mmol, 1.20 equiv), HATU (200 mg, 0.526 mmol, 1.10 equiv) and triethylamine (97 mg, 0.959 mmol, 4.00 equiv) in DMF (6 mL). The resulting mixture was stirred for 16 h at room temperature. The reaction was then quenched with water, extracted with ethyl acetate, washed with brine and concentrated. The residue was applied onto a silica gel column with EA/PE (45%) to yield 3-(6-bromo-4-oxoquinazolin-3(4H)-yl)-N-(4-fluorobenzyl)benzamide as an off-white solid.

Into a 50-mL round-bottom flask purged and maintained with an inert atmosphere of nitrogen, was placed a solution of 3-(6-bromo-4-oxoquinazolin-3(4H)-yl)-N-(4-fluorobenzyl)benzamide (170 mg, 0.376 mmol, 1.00 equiv), tert-butyl 4-(4,4,5,5-tetramethyl-1,3,2-dioxaborolan-2-yl)-1H-pyrazole-1-carboxylate (222 mg, 0.755 mmol, 2.00 equiv) and potassium carbonate (156 mg, 1.129 mmol, 3.00 equiv) and tetrakis(triphenylphosphine)palladium(0) (44 mg, 0.038 mmol, 0.10 equiv) in DMF/H₂O (5/0.5 mL). The resulting mixture was stirred for 16 h at 100° C. The reaction mixture was diluted with EA, washed with water and brine and concentrated. The residue was applied onto a silica gel column with EA/PE (25%) to yield tert-butyl 4-(3-((2-(4-fluorobenzylcarbamoyl)pyridin-4-yl)methyl)-4-oxo-3,4-dihydroquinazolin-6-yl)-1H-pyrazole-1-carboxylat as a light yellow solid.

Into a 25-mL round-bottom flask, was placed a solution of tert-butyl 4-(3-((2-(4-fluorobenzylcarbamoyl)pyridin-4-yl)methyl)-4-oxo-3,4-dihydroquinazolin-6-yl)-1H-pyrazole-1-carboxylate (100 mg, 0.185 mmol, 1.00 equiv) in DCM (5 mL). To the resulting mixture was then added trifluoroacetic acid (0.5 mL). The mixture was stirred for 2 h at room temperature. DCM was removed through rotavapor and NaHCO₃ (aq) was added. The filtrate cake isolated and washed with water and DCM to yield N-[(4-fluorophenyl)methyl]-3-[4-oxo-6-(1H-pyrazol-4-yl)quinazolin-3-yl]benzamide as a white solid.

¹H NMR (300 MHz, DMSO-d6) d 13.06 (s, 1H), 9.14 (t, J=6.0 Hz, 1H), 8.38 (s, 1H), 8.32 (d, J=1.9 Hz, 2H), 8.12 (dd, J=8.4, 2.2 Hz, 1H), 7.95-8.08 (m, 3H), 7.61-7.80 m, 3H), 7.29-7.41 (m, 2H), 7.06-7.19 m, 2H), 4.44 (d, J=5.9 Hz, 2H). m/z (MH⁺): 440.1.

### Example 39: Compound C5

### N-[(4-Fluorophenyl)methyl]-4-[[4-oxo-6-(1H-pyrazol-4-yl)quinazolin-3-yl]methyl]pyridine-2-carboxamide

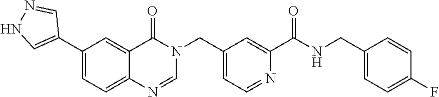 [SMILES: O=C(NCc1ccc(F)cc1)c1cc(Cn2cnc3ccc(-c4cn[nH]c4)cc3c2=O)ccn1]

Into a 50-mL round-bottom flask purged and maintained with an inert atmosphere of nitrogen, was placed a solution of 6-bromoquinazolin-4(3H)-one (518 mg, 2.302 mmol, 1.00 equiv), methyl 4-(bromomethyl)picolinate (556 mg, 2.417 mmol, 1.05 equiv), potassium carbonate (317 mg, 2.294 mmol, 3.00 equiv) in acetone (15 mL). The resulting mixture was stirred for 16 h at room temperature. The reaction mixture was concentrated. The residue was applied onto a silica gel column with EA (45%) to yield 4-((6-bromo-4-oxoquinazolin-3(4H)-yl)methyl)picolinate as a white solid.

Into a 50-mL round-bottom flask, was placed a solution of methyl 4-((6-bromo-4-oxoquinazolin-3(4H)-yl)methyl)picolinate (530 mg, 1.416 mmol, 1.00 equiv) in THE (8 mL). To the resulting mixture was added methanol (4 mL). To the resulting mixture was then added a solution of lithium hydroxide hydrate (16.9 mg, 0.403 mmol, 3.00 equiv) in H₂O (4 mL). The resulting mixture was stirred for 16 h at room temperature. THE was rotavaped and the mixture was adjusted to pH 7 with 1 M HCl (aq) and concentrated to yield 4-((6-bromo-4-oxoquinazolin-3(4H)-yl)methyl)picolinic acid as an off-white solid.

Into a 50-mL round-bottom flask purged and maintained with an inert atmosphere of nitrogen, was placed a solution of 4-((6-bromo-4-oxoquinazolin-3(4H)-yl)methyl)picolinic acid (300 mg, 0.833 mmol, 1.00 equiv), 4-fluorobenzylamine (136 mg, 1.087 mmol, 1.30 equiv), 1-hydroxybenzotrizole (169 mg, 1.251 mmol, 1.50 equiv), N-(3-dimethylaminopropyl)-N′-ethylcarbodiimide hydrochloride (240 mg, 1.252 mmol, 1.50 equiv) and triethylamine (253 mg, 2.500 mmol, 3.00 equiv) in DMF (8 mL). The resulting solution was stirred for 16 h at room temperature. The reaction was quenched with H₂O, extracted with ethyl acetate, washed with brine and concentrated. The residue was applied onto a silica gel column with MeOH/DCM (8%) to yield 4-((6-bromo-4-oxoquinazolin-3(4H)-yl)methyl)-N-(4-fluorobenzyl)picolinamide as a light yellow solid.

Into a 50-mL round-bottom flask purged and maintained with an inert atmosphere of nitrogen, was placed a solution of 4-((6-bromo-4-oxoquinazolin-3(4H)-yl)methyl)-N-(4-fluorobenzyl)picolinamide (240 mg, 0.514 mmol, 1.00 equiv), tert-butyl 4-(4,4,5,5-tetramethyl-1,3,2-dioxaborolan-2-yl)-1H-pyrazole-1-carboxylate (303 mg, 1.030 mmol, 2.00 equiv) and potassium carbonate (213 mg, 1.541 mmol, 3.00 equiv) and tetrakis(triphenylphosphine)palladium(0) (59 mg, 0.051 mmol, 0.10 equiv) in DMF/H₂O (6/0.6 mL). The resulting mixture was stirred for 16 h at 100° C. The reaction mixture was then diluted with ethyl acetate, washed with water and brine and concentrated. The residue was applied onto a silica gel column with EA/PE (25%) to yield tert-butyl 4-(3-((2-(4-fluorobenzylcarbamoyl)pyridin-4-yl)methyl)-4-oxo-3,4-dihydroquinazolin-6-yl)-1H-pyrazole-1-carboxylate as an off-white solid.

Into a 25-mL round-bottom flask, was placed a solution of tert-butyl 4-(3-((2-(4-fluorobenzylcarbamoyl)pyridin-4-yl)methyl)-4-oxo-3,4-dihydroquinazolin-6-yl)-1H-pyrazole-1-carboxylate (100 mg, 0.180 mmol, 1.00 equiv) in DCM (5 mL). To the resulting mixture was then added trifluoroacetic acid (0.5 mL). The resulting mixture was stirred for 2 h at room temperature. DCM was removed under reduced pressure and NaHCO₃ (aq) was added. The filtrate cake was isolated and washed with water and DCM to yield N-[(4-fluorophenyl)methyl]-4-[[4-oxo-6-(1H-pyrazol-4-yl)quinazolin-3-yl]methyl]pyridine-2-carboxamide as a white solid.

¹H NMR (300 MHz, DMSO-d6) d 13.03 (s, 1H), 9.36 (t, J=6.3 Hz, 1H), 8.54-8.61 (m, 1H), 8.52 (s, 1H), 8.35 (s, 1H), 8.26 (d, J=2.1 Hz, 1H), 8.09 (dd, J=8.5, 2.1 Hz, 1H), 8.02 (s, 1H), 7.90 (d, J=1.7 Hz, 1H), 7.68 (d, J=8.5 Hz, 1H), 7.51 (dd, J=5.1, 1.8 Hz, 1H), 7.23-7.35 (m, 2H), 7.00-7.14 (m, 2H), 5.29 (s, 2H), 4.40 (d, J=6.2 Hz, 2H). m/z (MH⁺): 455.1.

The following compounds were similarly prepared, following the procedure described in the Examples and Schemes herein, and selecting and substituting suitable reactants, as would be readily recognized by those skilled in the art.

### Example 40: Compound #57

### 3-[[5-Fluoro-4-oxo-6-(1H-pyrazol-4-yl)quinazolin-3-yl]methyl]-N-[(4-fluorophenyl)methyl]benzamide

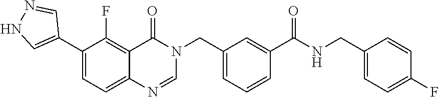 [SMILES: O=C(NCc1ccc(F)cc1)c1cccc(Cn2cnc3ccc(-c4cn[nH]c4)c(F)c3c2=O)c1]

¹H NMR (300 MHz, DMSO-d6) d 13.12 (s, 1H), 9.04 (t, J=6.0 Hz, 1H), 8.54 (s, 1H), 7.98-8.28 (m, 3H), 7.71-7.90 (m, 2H), 7.36-7.57 (m, 3H), 7.22-7.36 (m, 2H), 7.04-7.16 (m, 2H), 5.18 (s, 2H), 4.40 (d, J=5.8 Hz, 2H). m/z (MH⁺): 472.20

### Example 41: Compound #55

### 3-[[2-(3-Aminopyrrolidin-1-yl)-4-oxo-6-(1H-pyrazol-4-yl)quinazolin-3-yl]methyl]-N-[(4-fluorophenyl)methyl]benzamide

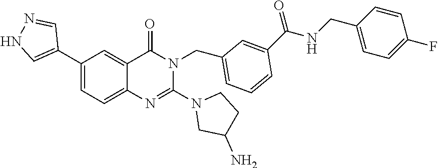 [SMILES: NC1CCN(c2nc3ccc(-c4cn[nH]c4)cc3c(=O)n2Cc2cccc(C(=O)NCc3ccc(F)cc3)c2)C1]

¹H NMR (300 MHz, DMSO-d6) d 9.02 (t, J=6.0 Hz, 1H), 8.12 (d, J=4.7 Hz, 3H), 7.89-8.06 (m, 4H), 7.68-7.78 (m, 2H), 7.39 (t, J=7.9 Hz, 2H), 7.23-7.35 (m, 3H), 7.03-7.16 (m, 2H), 5.30 (d, J=2.2 Hz, 2H), 4.39 (d, J=5.9 Hz, 2H), 3.80 (s, 1H), 3.70 (dd, J=11.7, 5.7 Hz, 1H), 3.43 (dd, J=11.2, 3.9 Hz, 2H), 3.26 (q, J=9.0, 8.4 Hz, 1H), 2.12 (dt, J=13.2, 6.5 Hz, 1H), 1.83 (d, J=12.3 Hz, 1H). m/z (MH⁺): 538.2

### Example 42: Compound #53

### 3-[(3-Methoxyphenyl)methyl]-6-(1H-pyrrolo[2,3-b]pyridin-4-yl)quinazolin-4-one

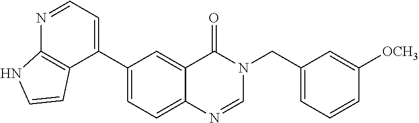 [SMILES: COc1cccc(Cn2cnc3ccc(-c4ccnc5[nH]ccc45)cc3c2=O)c1]

A mixture of 4-(4,4,5,5-tetramethyl-1,3,2-dioxaborolan-2-yl)-1H-pyrrolo[2,3-b]pyridine (314.3 mg, 1.29 mmol), 6-bromo-3-(3-methoxybenzyl)quinazolin-4(3H)-one (177.8 mg, 0.52 mmol), 2M aqueous potassium carbonate (0.52 ml, 1.03 mmol) in 1,4-dioxine (2 ml) was degassed with nitrogen, then treated with Pd(PPh₃)₄ (29.8 mg, 0.03 mmol). The reaction mixture was heated to 120° C. for 50 mins under microwave irradiation. It was cooled to room temperature, poured into aqueous NH₄Cl solution, extracted with EtOAc three times. The combined organic layer was dried, concentrated, and the residue was purified by flash column chromatography on silica gel (12 g, EtOAc/heptane: 0>>>40%>>>90%) to yield a white solid (165 mg, 83.7%).

¹H NMR (400 MHz, DMSO-d₆) δ 11.80-12.02 (m, 1H), 8.65 (s, 1H), 8.51 (d, J=2.02 Hz, 1H), 8.33 (d, J=5.05 Hz, 1H), 8.25 (dd, J=2.02, 8.59 Hz, 1H), 7.88 (d, J=8.59 Hz, 1H), 7.57-7.67 (m, 1H), 7.23-7.39 (m, 2H), 6.99 (s, 1H), 6.84-6.95 (m, 2H), 6.64 (dd, J=1.52, 3.54 Hz, 1H), 5.21 (s, 2H), 3.74 (s, 3H). m/z (MH⁺): 383.1

The following compound was similarly prepared, following the procedure described in the Examples and Schemes herein, and selecting and substituting suitable reactants, as would be readily recognized by those skilled in the art.

### Example 43: Compound #54

### 3-[(4-Fluoro-3-methoxy-phenyl)methyl]-6-(1H-pyrrolo[2,3-b]pyridin-4-yl)quinazolin-4-one

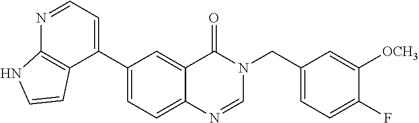 [SMILES: COc1cc(Cn2cnc3ccc(-c4ccnc5[nH]ccc45)cc3c2=O)ccc1F]

¹H NMR (400 MHz, DMSO-d₆) δ 11.85-12.02 (m, 1H), 8.67 (s, 1H), 8.52 (d, J=2.02 Hz, 1H), 8.33 (d, J=5.05 Hz, 1H), 8.25 (dd, J=2.02, 8.59 Hz, 1H), 7.88 (d, J=8.59 Hz, 1H), 7.61 (t, J=2.78 Hz, 1H), 7.27-7.36 (m, 2H), 7.19 (dd, J=8.34, 11.37 Hz, 1H), 6.95 (ddd, J=1.77, 4.17, 8.21 Hz, 1H), 6.59-6.71 (m, 1H), 5.20 (s, 2H), 3.84 (s, 3H). m/z (MH⁺): 401.1.

### Example 44: Compound #39

### 3-[(1R)-1-(3-Methoxyphenyl)ethyl]-6-(1H-pyrazol-4-yl)quinazolin-4-one

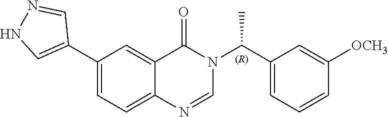

A mixture of 4-(4,4,5,5-tetramethyl-1,3,2-dioxaborolan-2-yl)-1H-pyrazole (78.6 mg, 0.41 mmol), (R)-6-bromo-3-(1-(3-methoxyphenyl)ethyl)quinazolin-4(3H)-one (58.2 mg, 0.16 mmol), 2M aqueous potassium carbonate (0.16 ml, 0.32 mmol) in 1,4-dioxine (2 ml) was degassed with nitrogen, then treated with Pd(PPh₃)₄ (9.4 mg, 0.008 mmol). The reaction mixture was heated to 120° C. for 65 mins under microwave irradiation. The resulting mixture was cooled to room temperature, poured into aqueous NH₄Cl solution, then extracted with EtOAc three times. The combined organic layer was dried, concentrated, and the residue was purified by flash column chromatography on silica gel (12 g, EtOAc/heptane: 0>>>60%>>>90%) to yield 3-[(1R)-1-(3-methoxyphenyl)ethyl]-6-(1H-pyrazol-4-yl)quinazolin-4-one as a white solid.

¹H NMR (400 MHz, CHLOROFORM-d) δ 8.48 (d, J=2.02 Hz, 1H), 8.01 (s, 2H), 7.87-7.97 (m, 2H), 7.70 (d, J=8.59 Hz, 1H), 7.29-7.36 (m, 1H), 6.96-7.02 (m, 1H), 6.90-6.95 (m, 1H), 6.79-6.89 (m, 1H), 6.31-6.45 (m, 1H), 3.79 (s, 3H), 1.84 (d, J=7.58 Hz, 3H). m/z (MH⁺): 347.1

The following compounds were similarly prepared, following the procedure described in the Examples and Schemes herein, and selecting and substituting suitable reactants, as would be readily recognized by those skilled in the art.

### Example 45: Compound #50

### 3-[(3-Chlorophenyl)methyl]-6-(1H-pyrazol-4-yl)quinazolin-4-one

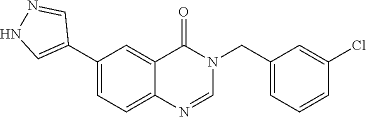 [SMILES: O=c1c2cc(-c3cn[nH]c3)ccc2ncn1Cc1cccc(Cl)c1]

¹H NMR (400 MHz, CHLOROFORM-d) δ 8.45 (d, J=2.02 Hz, 1H), 8.09 (s, 1H), 7.99 (s, 2H), 7.93 (d, J=2.02 Hz, 1H), 7.74 (d, J=8.08 Hz, 1H), 7.36 (s, 1H), 7.27-7.33 (m, 3H), 5.19 (s, 2H). m/z (MH⁺): 337.0

### Example 46: Compound #49

### 3-[(3-Chlorophenyl)methyl]-6-(3-pyridyl)quinazolin-4-one

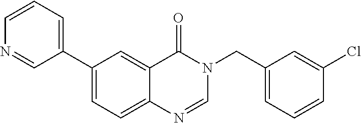 [SMILES: O=c1c2cc(-c3cccnc3)ccc2ncn1Cc1cccc(Cl)c1]

¹H NMR (400 MHz, CHLOROFORM-d) δ 8.95 (d, J=2.53 Hz, 1H), 8.65 (dd, J=1.26, 4.80 Hz, 1H), 8.55 (d, J=2.02 Hz, 1H), 8.14 (s, 1H), 7.96-8.05 (m, 2H), 7.82-7.88 (m, 1H), 7.43 (dd, J=4.80, 7.83 Hz, 1H), 7.37 (s, 1H), 7.27-7.33 (m, 3H), 5.21 (s, 2H). m/z (MH⁺): 348.0

### Example 47: Compound #48

### 3-[(3-Methoxyphenyl)methyl]-6-(2H-tetrazol-5-yl)quinazolin-4-one

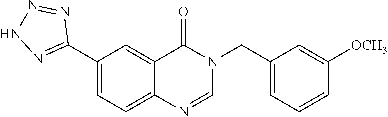 [SMILES: COc1cccc(Cn2cnc3ccc(-c4nn[nH]n4)cc3c2=O)c1]

¹H NMR (300 MHz, DMSO-d6) d 8.83 (d, J=2.0 Hz, 1H), 8.65 (s, 1H), 8.46 (dd, J=8.6, 2.1 Hz, 1H), 7.88 (d, J=8.5 Hz, 1H), 7.27 (t, J=7.9 Hz, 1H), 7.03-6.83 (m, 3H), 5.21 (s, 2H), 3.74 (s, 3H). m/z (MH⁺): 334.9

### Example 48: Compound #46

### 3-[(3-Methoxyphenyl)methyl]-6-(6-oxo-1H-pyridazin-4-yl)quinazolin-4-one

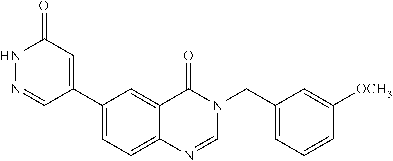 [SMILES: COc1cccc(Cn2cnc3ccc(-c4cn[nH]c(=O)c4)cc3c2=O)c1]

¹H NMR (300 MHz, DMSO-d6) d 13.18 (s, 1H), 8.66 (s, 1H), 8.48 (d, J=2.2 Hz, 1H), 8.38 (d, J=2.2 Hz, 1H), 8.24 (dd, J=8.5, 2.2 Hz, 1H), 7.81 (d, J=8.5 Hz, 1H), 7.33-7.19 (m, 2H), 7.00-6.95 (m, 1H), 6.95-6.83 (m, 2H), 5.19 (s, 2H), 3.73 (s, 3H). m/z (MH⁺): 361.2

### Example 49: Compound #45

### 6-(2-Aminopyrimidin-4-yl)-3-[(3-methoxyphenyl)methyl]quinazolin-4-one

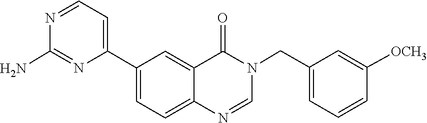 [SMILES: COc1cccc(Cn2cnc3ccc(-c4ccnc(N)n4)cc3c2=O)c1]

¹H NMR (300 MHz, DMSO-d6) d 8.89 (d, J=2.1 Hz, 1H), 8.62 (s, 1H), 8.48 (dd, J=8.6, 2.2 Hz, 1H), 8.36 (d, J=5.2 Hz, 1H), 7.79 (d, J=8.6 Hz, 1H), 7.31-7.21 (m, 2H), 7.01-6.96 (m, 1H), 6.96-6.83 (m, 2H), 6.78 (s, 2H), 5.19 (s, 2H), 3.73 (s, 3H). m/z (MH⁺): 360.2

### Example 50: Compound #44

### 3-[(3-Methoxyphenyl)methyl]-6-(2H-triazol-4-yl)quinazolin-4-one

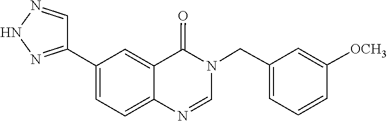 [SMILES: COc1cccc(Cn2cnc3ccc(-c4cn[nH]n4)cc3c2=O)c1]

¹H NMR (300 MHz, DMSO-d6) d 15.25 (s, 1H), 8.69-8.43 (m, 3H), 8.29 (dd, J=8.5, 2.1 Hz, 1H), 7.74 (d, J=8.5 Hz, 1H), 7.23 (t, J=7.9 Hz, 1H), 7.00-6.79 (m, 3H), 5.15 (s, 2H), 3.69 (s, 3H). m/z (MH⁺): 334.0

### Example 51: Compound #43

### 3-[(3-Methoxyphenyl)methyl]-6-(1H-pyrrolo[2,3-b]pyridin-3-yl)quinazolin-4-one

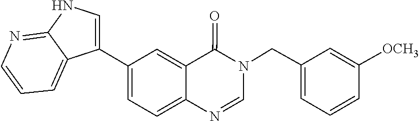 [SMILES: COc1cccc(Cn2cnc3ccc(-c4c[nH]c5ncccc45)cc3c2=O)c1]

¹H NMR (300 MHz, DMSO-d6) d 12.10 (s, 1H), 8.52 (s, 1H), 8.39 (d, J=2.1 Hz, 1H), 8.35-8.24 (m, 2H), 8.19 (dd, J=8.4, 2.2 Hz, 1H), 8.06 (d, J=2.7 Hz, 1H), 7.72 (d, J=8.5 Hz, 1H), 7.29-7.16 (m, 2H), 6.99-6.79 (m, 3H), 5.48 (brs, 1H), 5.16 (s, 2H), 3.70 (s, 3H); ¹⁹F NMR (282 MHz, DMSO-d6) d −74.78 (s, 3F). m/z (MH⁺): 383.1

### Example 52: Compound #42

### 3-[2-Hydroxy-1-(3-methoxyphenyl)ethyl]-6-(1H-pyrazol-4-yl)quinazolin-4-one

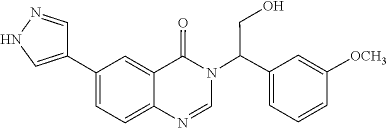 [SMILES: COc1cccc(C(CO)n2cnc3ccc(-c4cn[nH]c4)cc3c2=O)c1]

¹H NMR (300 MHz, DMSO-d6) d 13.02 (s, 1H), 8.48-8.18 (m, 3H), 8.15-7.94 (m, 2H), 7.63 (d, J=8.5 Hz, 1H), 7.25 (t, J=7.9 Hz, 1H), 7.04-6.78 (m, 3H), 6.02-5.83 (m, 1H), 5.27 (t, J=5.2 Hz, 1H), 4.37-4.20 (m, 1H), 4.05 (dt, J=11.4, 5.4 Hz, 1H), 3.70 (s, 3H). m/z (MH⁺): 363.0

### Example 53: Compound 41

### 3-[(3-Acetylphenyl)methyl]-6-(1H-pyrazol-4-yl)quinazolin-4-one

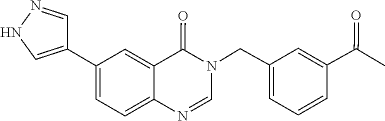 [SMILES: CC(=O)c1cccc(Cn2cnc3ccc(-c4cn[nH]c4)cc3c2=O)c1]

¹H NMR (400 MHz, CHLOROFORM-d) δ 10.20-10.50 (m, 1H), 8.44 (d, J=2.02 Hz, 1H), 8.08-8.19 (m, 1H), 7.99 (s, 3H), 7.88-7.96 (m, 2H), 7.74 (d, J=8.59 Hz, 1H), 7.60 (d, J=8.08 Hz, 1H), 7.45-7.53 (m, 1H), 5.27 (s, 2H), 2.61 (s, 3H). m/z (MH⁺): 345.1

### Example 54: Compound #40

### 3-[(5-Fluoro-2-methoxy-phenyl)methyl]-6-(1H-pyrazol-4-yl)quinazolin-4-one

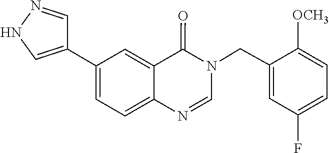 [SMILES: COc1ccc(F)cc1Cn1cnc2ccc(-c3cn[nH]c3)cc2c1=O]

¹H NMR (400 MHz, METHANOL-d₄) δ 8.71 (s, 1H), 8.33 (d, J=1.52 Hz, 1H), 8.14 (s, 2H), 8.01-8.09 (m, 1H), 7.66 (d, J=8.59 Hz, 1H), 7.20 (dd, J=3.03, 8.59 Hz, 1H), 6.92-7.09 (m, 2H), 5.13-5.20 (m, 2H), 3.86 (s, 3H). m/z (MH⁺): 351.0

### Example 55: Compound #38

### 3-[(3-Methoxyphenyl)methyl]-6-(5-oxo-1H-1,2,4-triazol-4-yl)quinazolin-4-one

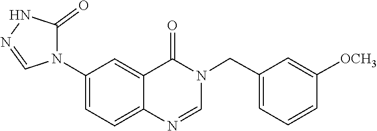 [SMILES: COc1cccc(Cn2cnc3ccc(-n4cn[nH]c4=O)cc3c2=O)c1]

¹H NMR (400 MHz, DMSO-d6) d 12.08 (s, 1H), 8.79-8.47 (m, 3H), 8.15 (d, J=8.6 Hz, 1H), 7.82 (d, J=8.8 Hz, 1H), 7.66-7.14 (m, 1H), 7.14-6.72 (m, 3H), 5.18 (s, 2H), 3.72 (s, 3H). m/z (MH⁺): 349.95

### Example 56: Compound #37

### 3-[(3-Methoxyphenyl)methyl]-6-(3-methyl-1H-pyrazol-4-yl)quinazolin-4-one

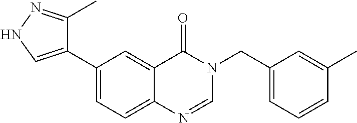 [SMILES: Cc1cccc(Cn2cnc3ccc(-c4c[nH]nc4C)cc3c2=O)c1]

¹H NMR (400 MHz, METHANOL-d₄) δ 8.56 (s, 1H), 8.29 (d, J=2.02 Hz, 1H), 8.05 (s, 1H), 7.97 (dd, J=2.02, 8.59 Hz, 1H), 7.71-7.80 (m, 2H), 7.26 (t, J=8.08 Hz, 1H), 6.93-7.01 (m, 2H), 6.86 (dd, J=2.02, 8.08 Hz, 1H), 5.24 (s, 2H), 3.76 (s, 3H), 2.52 (s, 3H). m/z (MH⁺): 347.10

### Example 57: Compound C4

### 3-(1,3-Benzodioxol-4-ylmethyl)-6-(1H-pyrazol-4-yl)quinazolin-4-one

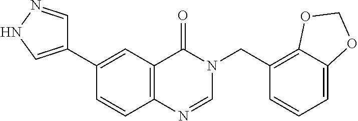 [SMILES: O=c1c2cc(-c3cn[nH]c3)ccc2ncn1Cc1cccc2c1OCO2]

To a suspension of 6-bromoquinazolin-4-ol (261.3 mg, 1.13 mmol) and K₂CO₃ (311.3 mg, 2.25 mmol) in acetonitrile (8 mL) was added 4-(bromomethyl)benzo[D][1,3]dioxole (254.9 mg, 1.13 mmol) and the resulting mixture was stirred at 70° C. for 4 h. The resulting mixture was diluted with 1N HCl and the top organic layer was separated. The organic layer was concentrated and the solid was washed with DCM three times, dried in vacuo to yield 3-(benzo[d][1,3]dioxol-4-ylmethyl)-6-bromoquinazolin-4(3H)-one as a residue, which was used directly for the next step reaction.

A mixture of 4-(4,4,5,5-tetramethyl-1,3,2-dioxaborolan-2-yl)-1H-pyrazole (126.4 mg, 0.65 mmol), 3-(benzo[d][1,3]dioxol-4-ylmethyl)-6-bromoquinazolin-4(3H)-one (117 mg, 0.33 mmol), 2M aqueous potassium carbonate (0.33 ml, 0.65 mmol) in 1,4-dioxine (2 ml) was degassed with nitrogen, then treated with Pd(PPh₃)₄ (18.8 mg, 0.016 mmol). The reaction mixture was heated to 130° C. for 65 mins under microwave irradiation. The resulting mixture was cooled to room temperature, poured into aqueous NH₄Cl solution, then extracted with EtOAc three times. The combined organic layer was dried, concentrated, and the residue was purified by Gilson HPLC to yield two pure fractions, both of which were combined and the solvent removed under reduced pressure to yield 3-(1,3-benzodioxol-4-ylmethyl)-6-(1H-pyrazol-4-yl)quinazolin-4-one as a white solid.

¹H NMR (DMSO-d6) Shift: 8.43 (s, 1H), 8.29 (s, 1H), 8.22 (br, 2H), 8.10 (dd, J=8.6, 2.0 Hz, 1H), 7.68 (d, J=8.6 Hz, 1H), 6.84-6.90 (m, 1H), 6.73-6.84 (m, 2H), 6.03 (s, 2H), 5.17 (s, 2H). m/z (MH)⁺: 347.10.

The following compounds were similarly prepared, following the procedure described in the Examples and Schemes herein, and selecting and substituting suitable reactants, as would be readily recognized by those skilled in the art.

### Example 58: Compound #34

### 3-[[4-Oxo-6-(1H-pyrazol-4-yl)quinazolin-3-yl]methyl]benzenesulfonamide

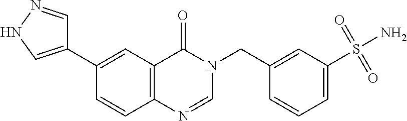 [SMILES: NS(=O)(=O)c1cccc(Cn2cnc3ccc(-c4cn[nH]c4)cc3c2=O)c1]

¹H NMR (300 MHz, DMSO-d6) d 13.02 (s, 1H), 8.53 (s, 1H), 8.43-8.23 (m, 2H), 8.15-7.95 (m, 2H), 7.78 (t, J=1.6 Hz, 1H), 7.72 (dt, J=7.5, 1.7 Hz, 1H), 7.66 (d, J=8.5 Hz, 1H), 7.62-7.47 (m, 2H), 7.32 (s, 2H), 5.25 (s, 2H). m/z (MH⁺): 382.0

### Example 59: Compound #35

### 3-[1-(3-Methoxyphenyl)propyl]-6-(1H-pyrazol-4-yl)quinazolin-4-one

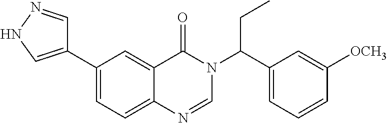 [SMILES: CCC(c1cccc(OC)c1)n1cnc2ccc(-c3cn[nH]c3)cc2c1=O]

¹H NMR (400 MHz, METHANOL-d₄) δ 8.38-8.44 (m, 2H), 8.15 (s, 2H), 8.03-8.09 (m, 1H), 7.65-7.71 (m, 1H), 7.25-7.36 (m, 1H), 7.03 (d, J=2.02 Hz, 2H), 6.83-6.94 (m, 1H), 5.99 (s, 1H), 3.74-3.82 (m, 3H), 2.21-2.51 (m, 2H), 1.00 (t, J=7.33 Hz, 3H). m/z (MH⁺): 361.10

### Example 60: Compound #33

### N-[3-[1-[4-Oxo-6-(1H-pyrazol-4-yl)quinazolin-3-yl]ethyl]phenyl]methanesulfonamide

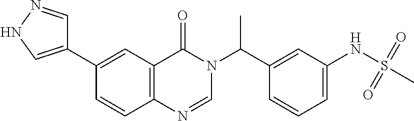 [SMILES: CC(c1cccc(NS(C)(=O)=O)c1)n1cnc2ccc(-c3cn[nH]c3)cc2c1=O]

¹H NMR (300 MHz, DMSO-d6) d 13.02 (s, 1H), 9.70 (s, 1H), 8.37-8.24 (m, 3H), 8.11-7.98 (m, 2H), 7.64 (d, J=8.5 Hz, 1H), 7.31 (dd, J=9.1, 6.8 Hz, 1H), 7.12 (d, J=7.4 Hz, 3H), 6.04 (q, J=7.3 Hz, 1H), 2.93 (s, 3H), 2.46 (q, J=3.0, 2.0 Hz, 2H), 1.80 (d, J=7.2 Hz, 3H). m/z (MH⁺): 409.95

### Example 61: Compound #31

### 3-[1-(3-Hydroxyphenyl)ethyl]-6-(1H-pyrazol-4-yl)quinazolin-4-one

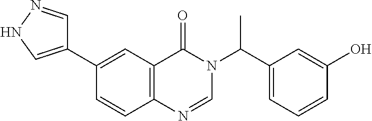 [SMILES: CC(c1cccc(O)c1)n1cnc2ccc(-c3cn[nH]c3)cc2c1=O]

¹H NMR (300 MHz, DMSO-d6) d 13.06 (s, 1H), 9.45 (s, 1H), 8.38 (s, 1H), 8.35-8.25 (m, 2H), 8.14-8.02 (m, 2H), 7.66 (d, J=8.5 Hz, 1H), 7.16 (t, J=7.8 Hz, 1H), 6.82 (d, J=7.8 Hz, 1H), 6.75-6.65 (m, 2H), 1.81 (d, J=7.2 Hz, 3H). m/z (MH⁺): 332.9

### Example 62: Compound #27

### 2-[3-[[4-Oxo-6-(1H-pyrazol-4-yl)quinazolin-3-yl]methyl]phenoxy]acetic acid

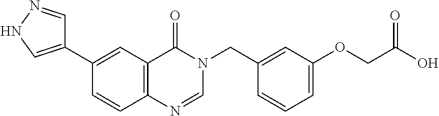 [SMILES: O=C(O)COc1cccc(Cn2cnc3ccc(-c4cn[nH]c4)cc3c2=O)c1]

¹H NMR (400 MHz, METHANOL-d₄) δ 8.49-8.58 (m, 1H), 8.40-8.46 (m, 1H), 8.14-8.27 (m, 2H), 8.06-8.14 (m, 1H), 7.72 (d, J=8.08 Hz, 1H), 7.24-7.35 (m, 1H), 6.99-7.06 (m, 2H), 6.89 (dd, J=2.02, 8.08 Hz, 1H), 5.26 (s, 2H), 4.66 (s, 2H). m/z (MH⁺): 377.1

### Example 63: Compound #26

### 2,2-Dideuterio-2-[3-[(1R)-1-[4-oxo-6-(1H-pyrazol-4-yl)quinazolin-3-yl]ethyl]phenoxy]acetic Acid

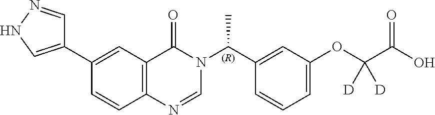

¹H NMR (400 MHz, METHANOL-d₄) δ 8.42-8.47 (m, 1H), 8.35-8.40 (m, 1H), 8.14-8.22 (m, 2H), 8.07-8.12 (m, 1H), 7.67-7.73 (m, 1H), 7.33 (s, 1H), 7.01-7.10 (m, 2H), 6.91 (dd, J=2.53, 8.08 Hz, 1H), 6.24 (q, J=7.41 Hz, 1H), 1.83-1.92 (m, 3H). m/z (MH⁺): 393.15.

### Example 64: Compound #24

### N-(1-Methylazetidin-3-yl)-3-[1-[4-oxo-6-(1H-pyrazol-4-yl)quinazolin-3-yl]ethyl]benzamide

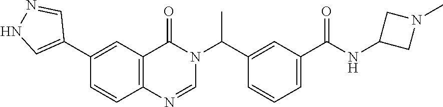 [SMILES: CC(c1cccc(C(=O)NC2CN(C)C2)c1)n1cnc2ccc(-c3cn[nH]c3)cc2c1=O]

¹H NMR (300 MHz, DMSO-d6) d 13.12 (d, J=18.6 Hz, 1H), 8.91 (d, J=6.9 Hz, 1H), 8.46-8.27 (m, 3H), 8.09 (dd, J=8.5, 2.2 Hz, 2H), 7.88 (d, J=2.0 Hz, 1H), 7.79 (d, J=7.6 Hz, 1H), 7.66 (d, J=8.5 Hz, 1H), 7.57 (d, J=7.7 Hz, 1H), 7.47 (t, J=7.7 Hz, 1H), 6.14 (q, J=7.2 Hz, 1H), 4.45 (p, J=7.1 Hz, 1H), 3.68 (t, J=7.4 Hz, 2H), 3.26-3.06 (m, 2H), 2.35 (s, 3H), 1.89 (d, J=7.3 Hz, 3H). m/z (MH⁺): 429.1

### Example 65: Compound #23

### N-Cyclopropyl-2,2-dideuterio-2-[3-[(1R)-1-[4-oxo-6-(1H-pyrazol-4-yl)quinazolin-3-yl]ethyl]phenoxy]acetamide

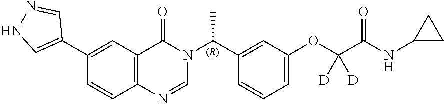

¹H NMR (400 MHz, MeOH) δ 8.43-8.48 (m, 1H), 8.34 (s, 1H), 8.16-8.28 (m, 2H), 8.08-8.14 (m, 1H), 7.70 (d, J=8.59 Hz, 1H), 7.31-7.41 (m, 1H), 7.09 (d, J=8.59 Hz, 1H), 7.04 (s, 1H), 6.94 (dd, J=2.27, 8.34 Hz, 1H), 6.24 (d, J=7.07 Hz, 1H), 2.60-2.71 (m, 1H), 1.89 (d, J=7.07 Hz, 3H), 0.69 (dd, J=2.02, 7.58 Hz, 2H), 0.46-0.54 (m, 2H).

### Example 66: Compound #20

### 3-[(3-Methoxyphenyl)methyl]-2-methyl-6-(1H-pyrazol-4-yl)quinazolin-4-one

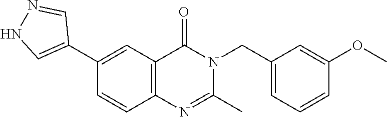 [SMILES: COc1cccc(Cn2c(C)nc3ccc(-c4cn[nH]c4)cc3c2=O)c1]

¹H NMR (400 MHz, METHANOL-d₄) δ 8.43-8.53 (m, 1H), 8.12-8.25 (m, 3H), 7.66-7.73 (m, 1H), 7.26-7.38 (m, 1H), 6.79-6.96 (m, 3H), 5.49 (s, 2H), 3.78 (s, 3H), 2.70 (s, 3H). m/z (MH⁺): 347.10

### Example 67: Compound #21

### 3-[(3-Methoxyphenyl)methyl]-6-(1H-pyrazol-4-yl)-2-(trifluoromethyl)quinazolin-4-one

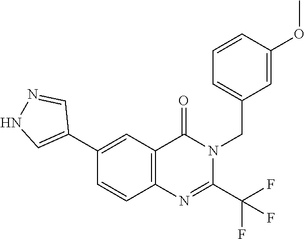 [SMILES: COc1cccc(Cn2c(C(F)(F)F)nc3ccc(-c4cn[nH]c4)cc3c2=O)c1]

¹H NMR (400 MHz, ACETONITRILE-d₃) δ 8.36-8.44 (m, 1H), 8.09 (brd, J=8.08 Hz, 1H), 8.00-8.35 (m, 2H), 7.77-7.84 (m, 1H), 7.19 (t, J=8.08 Hz, 1H), 6.78-6.85 (m, 1H), 6.66-6.76 (m, 2H), 5.33 (s, 2H), 3.70 (s, 3H). m/z (MH⁺): 401.1

### Example 68: Compound #19

### 6-(3-Amino-1H-pyrazol-4-yl)-3-[1-(3-methoxyphenyl)ethyl]quinazolin-4-one

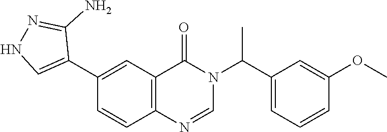 [SMILES: COc1cccc(C(C)n2cnc3ccc(-c4c[nH]nc4N)cc3c2=O)c1]

¹H NMR (400 MHz, DMSO-d6) d 8.65 (s, 1H), 8.36 (brs, 1H), 8.13 (d, J=8.4 Hz, 1H), 8.00 (brs, 1H), 7.85 (d, J=8.7 Hz, 1H), 7.28 (t, J=8.0 Hz, 1H), 7.08-7.17 (m, 2H), 6.81-6.91 (m, 1H), 6.48 (q, J=6.5 Hz, 1H), 5.25-5.55 (m, 1H), 4.80 (brs, 1H), 3.76 (s, 3H), 1.71 (d, J=6.5 Hz, 3H). m/z (MH⁺): 362.0

### Example 69: Compound C6

### 6-[2-(2-Methoxyethylamino)pyrimidin-4-yl]-3-[(3-methoxyphenyl)methyl]quinazolin-4-one

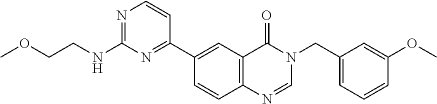

¹H NMR (400 MHz, DMSO-d6) d 9.10 (d, J=2.0 Hz, 1H), 8.71 (dd, J=8.6, 2.1 Hz, 1H), 8.59 (s, 1H), 8.17 (s, 1H), 7.75 (d, J=8.5 Hz, 1H), 7.59 (s, 1H), 7.25 (t, J=7.9 Hz, 1H), 6.99-6.82 (m, 3H), 6.48 (d, J=6.0 Hz, 1H), 5.18 (s, 2H), 3.72 (s, 3H), 3.62 (s, 2H), 3.53 (t, J=5.2 Hz, 2H), 3.29 (s, 3H). m/z (MH⁺): 418.0.

### Example 70: Compound #10

### 6-(2-Aminopyrimidin-4-yl)-3-[(3-methoxyphenyl)methyl]-2-methyl-quinazolin-4-one

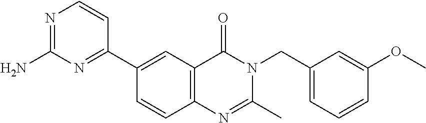 [SMILES: COc1cccc(Cn2c(C)nc3ccc(-c4ccnc(N)n4)cc3c2=O)c1]

¹H NMR (400 MHz, METHANOL-d₄) δ 9.07-9.18 (m, 1H), 8.61-8.68 (m, 1H), 8.33-8.42 (m, 1H), 7.75-7.83 (m, 1H), 7.57-7.65 (m, 1H), 7.20-7.35 (m, 1H), 6.84-6.92 (m, 1H), 6.80 (s, 2H), 5.42-5.49 (m, 2H), 3.76 (s, 3H), 2.60 (s, 3H). m/z (MH⁺): 374.10

### Example 71: Compound #7

### 5-Chloro-3-[(3-methoxyphenyl)methyl]-6-(1H-pyrazol-4-yl)quinazolin-4-one

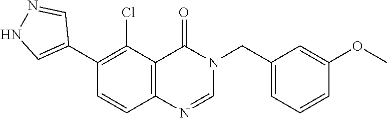 [SMILES: COc1cccc(Cn2cnc3ccc(-c4cn[nH]c4)c(Cl)c3c2=O)c1]

¹H NMR (400 MHz, METHANOL-d₄) δ 8.31-8.51 (m, 1H), 8.01-8.12 (m, 2H), 7.86-7.95 (m, 1H), 7.56-7.69 (m, 1H), 7.15-7.35 (m, 1H), 6.97 (br s, 2H), 6.84-6.90 (m, 1H), 5.19 (s, 2H), 3.78 (s, 3H), 3.35 (s, 3H). m/z (MH⁺): 367.0

### Example 72: Compound #5

### 5-Methoxy-3-[(3-methoxyphenyl)methyl]-6-(1H-pyrazol-4-yl)quinazolin-4-one

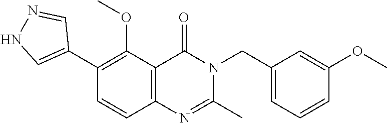 [SMILES: COc1cccc(Cn2c(C)nc3ccc(-c4cn[nH]c4)c(OC)c3c2=O)c1]

¹H NMR (400 MHz, METHANOL-d₄) δ 8.56 (s, 1H), 8.28 (br s, 1H), 8.09 (d, J=8.59 Hz, 1H), 7.48 (d, J=8.59 Hz, 1H), 7.26 (t, J=7.83 Hz, 1H), 6.94-7.06 (m, 2H), 6.86 (dd, J=1.77, 8.34 Hz, 1H), 5.21 (s, 2H), 3.76 (d, J=3.54 Hz, 6H). m/z (MH⁺): 363.3

### Example 73: Compound #11

### Methyl 2-(3-methoxyphenyl)-2-[4-oxo-6-(1H-pyrazol-4-yl)quinazolin-3-yl]acetate

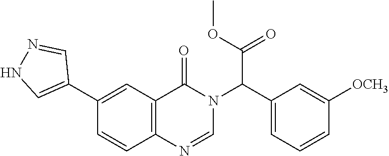 [SMILES: COC(=O)C(c1cccc(OC)c1)n1cnc2ccc(-c3cn[nH]c3)cc2c1=O]

To a mixture of 6-bromoisoindolin-1-one (237 mg, 1.12 mmol) and K₂CO₃ (308.9 mg, 2.34 mmol) in acetonitrile (5 ml) was added methyl 2-bromo-2-(3-methoxyphenyl)acetate (434.4 mg, 1.68 mmol) and the reaction mixture was stirred at room temperature for 16 h. The solid was filtered off and the filtrate was concentrated. The residue was purified by flash column chromatography on silica gel (4 g, EtOAc/heptane: 0>>>20%) to yield methyl 2-(6-bromo-4-oxoquinazolin-3(4H)-yl)-2-(3-methoxyphenyl)acetate as a light orange syrup (327 mg, 75%).

A 5 ml microwave vial was charged with methyl 2-(6-bromo-4-oxoquinazolin-3(4H)-yl)-2-(3-methoxyphenyl)acetate (306.2 mg, 0.76 mmol), tert-butyl 4-(4,4,5,5-tetramethyl-1,3,2-dioxaborolan-2-yl)-1H-pyrazole-1-carboxylate (223.4 mg, 0.76 mmol), Pd(PPh₃)₄ (43.9 mg, 0.038 mmol), 1,4-dioxane (2 ml) and K₂CO₃ (0.76 ml), the vial was capped and the mixture was heated under microwave irradiation at 130° C. for 65 mins. The solvent was removed under reduced pressure and the residue was re-dissolved in MeOH/acetonitrile (2.5 ml). The resulting residue was subjected to Gilson HPLC purification to yield methyl 2-(3-methoxyphenyl)-2-[4-oxo-6-(1H-pyrazol-4-yl)quinazolin-3-yl]acetate as a white solid.

¹H NMR (400 MHz, METHANOL-d₄) δ 8.36-8.50 (m, 1H), 8.11 (s, 3H), 7.89 (s, 1H), 7.61-7.73 (m, 1H), 7.35-7.48 (m, 1H), 7.03 (s, 3H), 6.59 (s, 1H), 3.86 (s, 3H), 3.81 (s, 3H). m/z (MH⁺): 391.2.

### Example 74: Compound #12

### (3-Methoxyphenyl)-2-[4-oxo-6-(1H-pyrazol-4-yl)quinazolin-3-yl]acetic acid

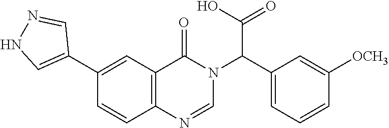 [SMILES: COc1cccc(C(C(=O)O)n2cnc3ccc(-c4cn[nH]c4)cc3c2=O)c1]

To a solution of methyl 2-(3-methoxyphenyl)-2-(4-oxo-6-(1H-pyrazol-4-yl)quinazolin-3(4H)-yl)acetate (65 mg, 0.17 mmol) in THF/MeOH was added 1N NaOH (0.83 mL) and the mixture was stirred at room temperature for 2 h. The solvent was removed under reduced pressure and the residue was acidified with 1N HCl and the precipitate was washed with MeOH, then dried in vacuo to yield (3-methoxyphenyl)-2-[4-oxo-6-(1H-pyrazol-4-yl)quinazolin-3-yl]acetic acid as a white solid.

¹H NMR (400 MHz, METHANOL-d₄) δ 8.46 (s, 1H), 8.11 (br s, 2H), 8.06 (br d, J=8.59 Hz, 1H), 7.98 (s, 1H), 7.64 (d, J=8.59 Hz, 1H), 7.34 (t, J=7.83 Hz, 1H), 7.03-7.09 (m, 2H), 6.94 (br d, J=9.09 Hz, 1H), 6.66 (s, 1H), 3.80 (s, 3H). m/z (MH⁺): 377.3

The following compounds were similarly prepared, following the procedure described in the Examples and Schemes herein, and selecting and substituting suitable reactants, as would be readily recognized by those skilled in the art.

### Example 75: Compound #9

### 8-Fluoro-3-[(3-methoxyphenyl)methyl]-6-(1H-pyrazol-4-yl)quinazolin-4-one

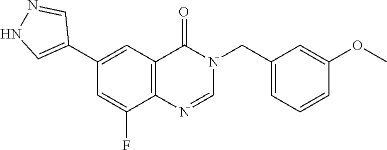 [SMILES: COc1cccc(Cn2cnc3c(F)cc(-c4cn[nH]c4)cc3c2=O)c1]

¹H NMR (400 MHz, CHLOROFORM-d) δ 8.19-8.26 (m, 1H), 8.11 (s, 1H), 7.94-8.07 (m, 2H), 7.61-7.67 (m, 1H), 7.29 (s, 1H), 6.79-6.98 (m, 3H), 5.20 (s, 2H), 3.79 (s, 3H). m/z (MH⁺): 351.3

### Example 76: Compound #6

### 7-Methoxy-3-[(3-methoxyphenyl)methyl]-6-(1H-pyrazol-4-yl)quinazolin-4-one

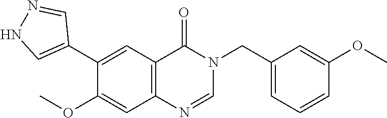 [SMILES: COc1cccc(Cn2cnc3cc(OC)c(-c4cn[nH]c4)cc3c2=O)c1]

¹H NMR (400 MHz, METHANOL-d₄) δ 8.48-8.54 (m, 1H), 8.36-8.41 (m, 1H), 8.18 (s, 2H), 7.23-7.30 (m, 1H), 7.18 (s, 1H), 6.93-7.01 (m, 2H), 6.87 (dd, J=2.02, 8.08 Hz, 1H), 5.21 (s, 2H), 4.05 (s, 3H), 3.77 (s, 3H). m/z (MH⁺): 363.3.

### Biological Example 1

### hGRK2 LANCE Ultra In Vitro Assay

G-protein coupled receptor kinases (GR Kinases) desensitize activated G-protein coupled receptors (GPCRs), by phosphorylation of cytoplasmic loops or carboxyl-terminal tails of GPCRs. GRK2 is one of the 6 different GR kinases and is implicated in heart failure and diabetes.

The purpose of the LANCE Ultra assay (http://www.perkinelmer.com/ResourcesRsources/ApplicationSupportKnowledgease/LANCE/lance.xhtml) is used to test inhibitors against GRK2 in its inactive state. This assay is sensitive and requires as low as 10 nM enzyme, in a total volume of 10 μL. In addition, the ATP concentration can be varied over a broad range, without interfering with the assay or changing the assay condition. This property makes it easy to characterize very potent ATP-competitive inhibitors by increasing ATP concentrations. Testing inhibitors routinely at both high and low ATP concentrations also enables identification of potential non-ATP competitive inhibitors.

Paroxetine was used as the reference compound in this assay. The IC₅₀ value determined by the LANCE Ultra assay was 8.3 μM, which is in good agreement with the literature value (THAL, D. M., et al. “Paroxetine is a direct inhibitor of G protein-coupled receptor kinase 2 and increases myocardial contractility”, ACS Chemical Biology, 2012, pp 1830, Vol. 7).

This assay measures IC₅₀ values of test compounds (inhibitors) by monitoring GRK2 enzymatic activity at varying inhibitor concentrations.

Test compounds were dissolved in DMSO at 1 mM and were 3-fold serial diluted. The compound DMSO solutions were then added (100 nL) into a plate well using an acoustic dispenser. To each well was then added 20 nM GRK2 (5 μL) in assay buffer (20 mM HEPES, pH 7.5, 10 mM MgCl₂, 0.001% Tween-20®). The plate was sealed and centrifuged at 1000 rpm for 1 min.

The plate and wells containing a mixture of GRK2 and test compound were incubated at ambient temperature for 30 min (prior to initializing the enzymatic reaction).

Enzyme reactions were initiated by the addition of 4.9 μL Substrates/Eu-Ab mix to each well. For assays at low ATP concentration (1×Kₘ value), the Substrate/Eu-Ab mix contains 60 μM ATP, 400 nM ULight-peptide (LANCE® Ultra ULight™-DNA Topoisomerase 2-alpha (Thr1342) Peptide), and 8 nM Eu-Ab (LANCE® Ultra Europium-anti-phospho-DNA Topoisomerase 2-alpha (Thr1342)) in the assay buffer. For assays at high ATP concentration (20×Kₘ value), the Substrate/Eu-Ab mix contains 1.2 mM ATP, 400 nM ULight-peptide, and 8 nM Eu-Ab in the assay buffer. Final concentrations of reagents in the assays were as follows: 20 mM HEPES, pH 7.5; 10 mM MgCl₂; 0.001% Tween-20® (w/v); 30 or 600 μM ATP; 200 nM ULight-peptide; 4 nM Eu-Ab; 10 nM GRK2; and 1% DMSO.

The plates were sealed and centrifuged at 1000 rpm for 1 min. For reactions at low ATP concentration (30 μM), reaction mixtures were incubated at ambient temperature for 120 min. For reactions at high ATP concentration (600 μM), reaction mixtures were incubated at ambient temperature for 60 min.

The enzyme reactions were quenched by addition of 10 μL of 12 mM EDTA in 1× LANCE detection solution to each well. The plates were then incubated at ambient temperature for 30 min. Time-resolved fluorescence signal of reactions were read on an EnVision or PHERAstar plate reader with the following parameters: Excitation wavelength=337 nm; emission wavelength (donor)=620 nm; emission wavelength (acceptor)=665 nm.

To calculate IC₅₀ values, compounds were serially diluted 3-fold and tested in 11-point dose responses. The raw HTRF data were converted to % active as follows:

% active=(sample−NC)/(PC−NC)*100

where NC is the mean of negative control (reactions without GRK2), and PC is the mean of positive control (reactions with GRK2 but without inhibitor). IC₅₀ values were determined from a 4-parameter fit, using the following equation:

Y=Bottom+(Top−Bottom)/(1+10^{((Log IC50-X)*Hill slope)}),

where X=log₁₀ of the compound concentration.

### Biological Example 2

### GRK2 Transcreener Assay

Test compounds were dissolved in 100% DMSO and then added into a 384-well Corning 3676 plate using an acoustic dispenser. Positive and negative control wells received an equal volume of DMSO. The final DMSO concentration in the assay is 1%.

15 μM ATP (6.5 uL) in assay buffer (10 mM HEPES, pH 7.5, 2 mM DTT, 5 mM MgCl₂, 0.005% Brij™-35) was added to each well, followed by the addition of 1.5 μL of 1 mM peptide substrate (amino acid sequence: MEFTEAESNMNDLVSEYQ). The plate was placed in centrifuge equipped with a spin-bucket rotor and spun for 1 min at 1000 rpm.

GRK2 enzymatic reactions were initiated with the addition of 2 μL/well of 50 nM GRK2 in assay buffer. Plates were centrifuged for 1 min at 1000 rpm. For negative control wells, the order of reagent addition was reversed: 10 μL/well ADP detection mix (see below) was added first, followed by the addition of 2 μL/well of the GRK2 solution. Reaction mixtures were incubated at ambient temperature for 2 hours. Final concentrations of reagents in the assays were as follows:
10 mM HEPES, pH 7.5
2 mM DTT
5 mM MgCl₂
0.005% Brij™-35 (w/v)
10 uM ATP
150 uM MEFTEAESNMNDLVSEYQ peptide
10 nM GRK2
1% DMSO

Following incubation, the reactions were quenched with 10 μL/well of the Transcreener ADP detection mix. The detection mix contains 4 nM Alexa633 tracer, 11.8 μg/mL anti-ADP antibody and 1× “stop & detect” buffer (BellBrook Labs, catalog number 3010-10K). The plates were then centrifuged for 1 min at 1000 rpm.

Fluorescence polarization values of the reaction mixtures were read on a Safire II plate reader after a 60 min incubation at ambient temperature. Excitation wavelength=590 nm; emission wavelength=650 nm.

To calculate IC₅₀ values, compounds were serially diluted 2-fold and tested in 11-point dose responses. The fluorescence polarization data were converted to % activity as follows:

% activity=(sample−NC)/(PC−NC)*100

where NC is the mean of negative control (ADP detection mix added prior to GRK2 addition), and PC is the mean of positive control (GRK2 reaction without inhibitor). IC₅₀ values are determined from a 4-parameter fit, using the following equation:

Y=Bottom+(Top−Bottom)/(1+10^{((Log IC50-X)*Hill slope)}),

where X=log₁₀ of the compound concentration.

Representative compounds of the present invention were tested according to the procedure described in Biological Example 1 and Biological Example 2, above, with results as listed in Table 2 below. Results are reported as the IC₅₀ value. Variability for the functional assay was typically within 2-fold.

TABLE 2
GRK2 Biological Activity, Compounds of Formula (I)

| | GRK2 Lancer | |
|---|---|---|
| ID No. | IC₅₀ (μM) | GRK2 IC₅₀ (μM) |
| 1 | 0.005 | |
| 2 | 0.006 | ~0.01; 0.011 |
| 3 | 0.007 | |
| 4 | 0.009 | |
| 5 | 0.009 | |
| 6 | 0.016 | |
| 7 | 0.016 | |
| 8 | 0.029 | |
| 9 | 0.093 | |
| 10 | 0.371 | |
| 11 | 1.41 | |
| 12 | 2.55 | |
| 13 | | <0.01 |
| 14 | | 2.11 |
| 15 | | 5.59 |
| 17 | | 1.31 |
| 19 | | >50 |
| 20 | | 0.014 |
| 21 | | 0.425 |
| 22 | | 4.11 |
| 23 | | 0.035 |
| 24 | | 0.096 |
| 25 | | 2.34 |
| 26 | | 0.143 |
| 27 | | 0.088 |
| 28 | | 0.446 |
| 29 | | 0.054 |
| 30 | | 0.012 |
| 31 | | 0.008 |
| 32 | | 0.157 |
| 33 | | 0.033 |
| 34 | | 0.030 |
| 35 | | 0.122 |
| 37 | | 0.066 |
| 38 | | 0.129 |
| 39 | | 0.009; 0.017 |
| 40 | | 1.72 |
| 41 | | 0.065 |
| 42 | | 0.025 |
| 43 | | 0.757 |
| 44 | | 0.100 |
| 45 | | 0.271 |
| 46 | | 1.46 |
| 48 | | >50 |
| 49 | | >50 |
| 50 | | 0.075 |
| 52 | | >19 |
| 53 | | 2.45 |
| 54 | | 12.29 |
| 55 | | 0.014 |
| 57 | | <0.01 |
| 60 | | 0.009 |
| 61 | | 0.007 |
| 62 | | 0.018 |
| 63 | | 0.065 |
| 64 | | 0.206 |
| 65 | | 0.078 |
| 66 | | 22.18 |
| 67 | | 2.75 |
| 68 | | 0.472 |
| 69 | | 0.278 |
| 70 | | <0.01; <0.01 |
| 72 | | <0.01 |
| 77 | | >20 |
| 78 | | 0.801 |
| 79 | | 0.104; 0.087 |
| 80 | | >100 |
| 81 | | ~25 |
| 82 | | >100 |
| 83 | | >100 |
| 84 | | 4.125 |
| C1 | | 1.63 |
| C2 | | 4.344 |
| C3 | | ~20 |
| C4 | | 0.093 |
| C5 | | 0.212 |
| C6 | | >50 |

### Biological Example 3—Prophetic Example

### GLP-1 Mediated Beta-Arrestin Recruitment Assay

PathHunter® eXpress GLP1R CHO-K1 β-Arrestin cells are plated at 6000/well in a 384-well PDL white and opaque plate in F12 medium with 10% FBS, 0.3 mg/ml hygromycin, and 0.8 mg/ml G418. The plate is maintained in a humidified incubator at 37° C. and 5% CO₂ for 2 days before the experiment. On the day of the experiment, the cells are washed once with the Assay Buffer (HBSS with calcium and magnesium, 20 mM HEPES, and 0.1% fatty-acid free BSA). Test compound or vehicle (DMSO) is added to the cells at the indicated concentrations, 10 min prior to the addition of GLP-1. The final DMSO concentration is 0.1%. After 90 min incubation at 37° C., the detection reagent is added the cells, followed by 60 min incubation at the room temperature. The plate is read on MicroBeta LumiJet (PerkinElmer, Waltham, Mass.).

### Formulation Example 1

### Solid, Oral Dosage Form—Prophetic Example

As a specific embodiment of an oral composition, 100 mg of the Compound #3 (prepared as in Example 14) is formulated with sufficient finely divided lactose to provide a total amount of 580 to 590 mg to fill a size O hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

Throughout this application, various publications are cited. The disclosure of these publications is hereby incorporated by reference into this application to describe more fully the state of the art to which this invention pertains.

1. A compound of formula (I) 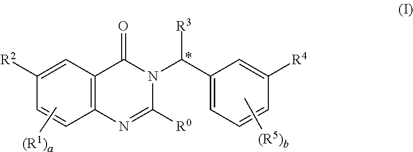
wherein
R⁰ is selected from the group consisting of hydrogen, C₁₋₄alkyl, fluorinated C₁₋₂alkyl, C₁₋₄alkoxy, fluorinated C₁₋₂alkoxy and 4 to 7 membered, nitrogen containing, saturated heterocyclyl;
wherein the 4 to 7 membered, nitrogen containing, saturated heterocyclyl is optionally substituted with one or more substituents independently selected from the group consisting of hydroxy and NR^{X}R^{Y}; wherein R^{X} and R^{Y} are each independently selected from the group consisting of hydrogen and C₁₋₄alkyl;
a is an integer from 0 to 3;
each R¹ is independently selected from the group consisting of halogen, hydroxy, C₁₋₄alkyl, fluorinated C₁₋₂alkyl, C₁₋₄alkoxy, fluorinated C₁₋₂alkoxy and cyano;
R² is selected from the group consisting of 5 to 6 membered heteroaryl and 1H-pyrrolo[2,3-b]pyridin-3-yl; wherein the 5 to 6 membered heteroaryl is optionally substituted with one or more substituents independently selected from the group consisting of halogen, C₁₋₄alkyl, fluorinated C₁₋₂alkyl, oxo and NR^{A}R^{B}; wherein R^{A} and R^{B} are each independently selected from the group consisting of hydrogen and C₁₋₂alkyl;
R³ is selected from the group consisting of hydrogen, —C₁₋₄alkyl, —C₁₋₄alkoxy, —(C₁₋₂alkyl)-OH, —(C₁₋₂alkyl)-NR^{C}R^{D}, —(C₁₋₂alkyl)-SO₂—(C₁₋₂alkyl), —CO₂H, —C(O)O—(C₁₋₂alkyl) and tetrahydropyran-4-yl-1,1-dioxide; wherein R^{C} and R^{D} are each independently selected from the group consisting of hydrogen and C₁₋₂alkyl;
R⁴ is selected from the group consisting of hydrogen, halogen, hydroxy, C₁₋₄alkyl, fluorinated C₁₋₂alkyl, C₁₋₄alkoxy, fluorinated C₁₋₂alkoxy, —(C₁₋₂alkyl)-CO₂H, —(C₁₋₂alkyl)-C(O)O—(C₁₋₄alkyl), —O—C₂₋₄alkynyl, —O—(C₁₋₂alkyl)-C(O)OH, —O—(C₁₋₂alkyl)-C(O)O—(C₁₋₂alkyl), —O—(C₁₋₂alkyl)-O—(C₃₋₅cycloalkyl), —O—(C₁₋₂alkyl)-C(O)-morpholine, —O—(C₁₋₂alkyl)-C(O)—NR^{E}R^{F}, —O—(C₁₋₂alkyl)-C(O)—NH—(C₃₋₅cycloalkyl), —O—(C₁₋₂alkyl)-SO₂—(C₁₋₂alkyl), —O—(C₃₋₆cycloalkyl), —O-phenyl, —O— benzyl, —O-azetidin-3-yl, —O-(1-methyl-azetidin-3-yl), —O-pyrrolidin-3-yl, —O-(1-methyl-pyrrolidin-3-yl), —O-piperidin-4-yl, —O-(1-methyl-piperidin-4-yl), —C(O)—(C₁₋₂alkyl), —C(O)—NR^{E}R^{F}, —C(O)—NH—(C₂₋₄alkynyl), —C(O)—NH—(C₂alkyl)-CO₂H, —C(O)—NH—(C₂alkyl)-C(O)O—(C₁₋₂alkyl), —C(O)—NH-(phenyl), —C(O)—NH-(benzyl), —C(O)—NH—(C₃₋₈cycloalkyl), —C(O)—NH-(pyridinyl), —C(O)—NH—(CH₂CH₂-morpholin-4-yl), —C(O)—NH-(azetidin-3-yl), —C(O)—NH-(1-methyl-azetidin-3-yl), —C(O)—NH-pyrrolidin-3-yl, —C(O)—NH-(1-methyl-pyrrolidin-3-yl), —C(O)—NH-piperidin-4-yl, —C(O)—NH-(1-methyl-piperidin-4-yl), —NH—SO₂—(C₁₋₂alkyl), —S—(C₁₋₄alkyl), —SO—(C₁₋₄alkyl), —SO₂—(C₁₋₄alkyl), —SO₂—NR^{E}R^{F}, and oxazol-2-yl;
wherein the phenyl or benzyl, whether alone or as part of a substituent group, is optionally substituted with one to two substituents independently selected from the group consisting of halogen, C₁₋₄alkyl and C₁₋₄alkoxy;
and wherein R^{E} and R^{F} are each independently selected form the group consisting of hydrogen and C₁₋₄alkyl;
b is an integer from 0 to 4;
each R⁵ is independently selected from the group consisting of halogen, C₁₋₄alkyl and C₁₋₄alkoxy;
provided than when R⁰ is hydrogen or methyl, a is an integer from 0 to 1, R¹, when present, is 8-methyl, R³ is hydrogen, R⁴ is methoxy, and b is 0, then R² is other than pyrazol-4-yl or imidazol-1-yl;
or an isotopologue or pharmaceutically acceptable salt thereof.

2. The compound of claim 1, wherein
R⁰ is selected from the group consisting of hydrogen, C₁₋₄alkyl, fluorinated C₁₋₂alkyl, C₁₋₄alkoxy, fluorinated C₁₋₂alkoxy and 5 to 6 membered, nitrogen containing, saturated heterocyclyl;
wherein the 5 to 6 membered, nitrogen containing, saturated heterocyclyl is optionally substituted with one to two substituents independently selected from the group consisting of hydroxy and NR^{X}R^{Y}; wherein R^{X} and R^{Y} are each independently selected from the group consisting of hydrogen and C₁₋₂alkyl;
a is an integer from 0 to 2;
each R¹ is independently selected from the group consisting of halogen, hydroxy, C₁₋₂alkyl, fluorinated C₁₋₂alkyl, C₁₋₂alkoxy, fluorinated C₁₋₂alkoxy and cyano;
R² is selected from the group consisting of 5 to 6 membered heteroaryl and 1H-pyrrolo[2,3-b]pyridin-3-yl; wherein the 5 to 6 membered heteroaryl is optionally substituted with one to two substituents independently selected from the group consisting of halogen, C₁₋₄alkyl, fluorinated C₁₋₂alkyl, oxo and NR^{A}R^{B}; wherein R^{A} and R^{B} are each independently selected from the group consisting of hydrogen and C₁₋₂alkyl;
R³ is selected from the group consisting of hydrogen, —C₁₋₄alkyl, —C₁₋₂alkoxy, —(C₁₋₂alkyl)-OH, —(C₁₋₂alkyl)-NR^{C}R^{D}, —(C₁₋₂alkyl)-SO₂—(C₁₋₂alkyl), —CO₂H, —C(O)O—(C₁₋₂alkyl) and tetrahydropyran-4-yl-1,1-dioxide; wherein R^{C} and R^{D} are each independently selected from the group consisting of hydrogen and C₁₋₂alkyl;
R⁴ is selected from the group consisting of hydrogen, halogen, hydroxy, C₁₋₄alkoxy, fluorinated C₁₋₂alkoxy, —O—(C₁₋₂alkyl)-CO₂H, —O—(C₁₋₂alkyl)-C(O)O—(C₁₋₄alkyl), —O—(C₁₋₂alkyl)-C(O)-morpholine, —O—(C₁₋₂alkyl)-C(O)—NR^{E}R^{F}, —O—(C₁₋₂alkyl)-C(O)—NH—(C₃₋₅cycloalkyl), —O—(C₁₋₂alkyl)-SO₂—(C₁₋₂alkyl), —O—(C₃-6cycloalkyl), —O-phenyl, —O-benzyl, —O-azetidin-3-yl, —O-(1-methyl-azetidin-3-yl), —O-pyrrolidin-3-yl, —O-(1-methyl-pyrrolidin-3-yl), —O-piperidin-4-yl, —O-(1-methyl-piperidin-4-yl), —C(O)—(C₁₋₂alkyl), —C(O)—NR ERF, —C(O)—NH-(phenyl), —C(O)—NH-(benzyl), —C(O)—NH—(C₃₋₈cycloalkyl), —C(O)—NH-(pyridinyl), —C(O)—NH—(CH₂CH₂-morpholin-4-yl), —C(O)—NH-(azetidin-3-yl), —C(O)—NH-(1-methyl-azetidin-3-yl), —C(O)—NH-pyrrolidin-3-yl, —C(O)—NH-(1-methyl-pyrrolidin-3-yl), —C(O)—NH-piperidin-4-yl, —C(O)—NH-(1-methyl-piperidin-4-yl), —NH—SO₂—(C₁₋₂alkyl), —S—(C₁-4alkyl), —SO—(C₁₋₄alkyl), —SO₂—(C₁₋₄alkyl), —SO₂—NR^{E}R^{F} and oxazol-2-yl;
wherein the phenyl or benzyl, whether alone or as part of a substituent group, is optionally substituted with one to two substituents independently selected from the group consisting of halogen, C₁₋₂alkyl and C₁₋₂alkoxy; and wherein R^{E} and R^{F} are each independently selected form the group consisting of hydrogen and C₁₋₂alkyl;
b is an integer from 0 to 2;
each R⁵ is independently selected from the group consisting of halogen, C₁₋₂alkyl and C₁₋₄alkoxy;
provided than when R⁰ is hydrogen or methyl, a is an integer from 0 to 1, R¹, when present, is 8-methyl, R³ is hydrogen, R⁴ is methoxy, and b is 0, then R² is other than pyrazol-4-yl or imidazol-1-yl;
or an isotopologue or pharmaceutically acceptable salt thereof.

3. The compound of claim 2, wherein
R⁰ is selected from the group consisting of hydrogen, C₁₋₂alkyl, fluorinated C₁₋₂alkyl and pyrrolidin-1-yl; wherein the pyrrolidin-1-yl is optionally substituted with NR^{X}R^{Y}; wherein R^{X} and R^{Y} are each independently selected from the group consisting of hydrogen and C₁₋₂alkyl;
a is an integer from 0 to 1;
R¹ is selected from the group consisting of halogen, hydroxy, C₁₋₂alkoxy, fluorinated C₁₋₂alkoxy and cyano;
R² is selected from the group consisting of pyrazolyl, pyrimidinyl, pyridinyl, pyridazinyl, triazolyl, tetrazolyl and 1H-pyrrolo[2,3-b]pyridin-3-yl; wherein the pyrazolyl, pyrimidinyl, pyridinyl, pyridazinyl, triazolyl or tetrazolyl is optionally substituted with a substituent selected from the group consisting of halogen, C₁₋₂alkyl, fluorinated C₁₋₂alkyl, oxo and NR^{A}R^{B}; wherein R^{A} and R^{B} are each independently selected from the group consisting of hydrogen and C₁₋₂alkyl;
R³ is selected from the group consisting of hydrogen, —C₁₋₂alkyl, —(C₁₋₂alkyl)-OH, —(C₁₋₂alkyl)-NR^{C}R^{D}, —(C₁₋₂alkyl)-SO₂—(C₁₋₂alkyl), —CO₂H, —C(O)O—(C₁₋₂alkyl) and tetrahydropyran-4-yl-1,1-dioxide; wherein R^{C} and R^{D} are each independently selected from the group consisting of hydrogen and C₁₋₂alkyl;
R⁴ is selected from the group consisting of hydrogen, halogen, hydroxy, C₁₋₂alkoxy, fluorinated C₁₋₂alkoxy, —O—(C₁₋₂alkyl)-C(O)OH, —O—(C₁₋₂alkyl)-C(O)O—(C₁₋₂alkyl), —O—(C₁₋₂alkyl)-C(O)—NH—(C₃₋₅cycloalkyl), —O—(C₁₋₂alkyl)-SO₂—(C₁₋₂alkyl), —O-phenyl, —O-benzyl, —O-azetidin-3-yl, —O-(1-methyl-azetidin-3-yl), —C(O)—(C₁₋₂alkyl), —C(O)—NH-(phenyl), —C(O)—NH-(benzyl), —C(O)—NH-(azetidin-3-yl), —C(O)—NH-(1-methyl-azetidin-3-yl), —NH—SO₂—(C₁₋₂alkyl), and oxazol-2-yl; wherein the phenyl or benzyl, whether alone or as part of a substituent group, is optionally substituted with halogen;
b is an integer from 0 to 2;
each R⁵ is independently selected from the group consisting of halogen and C₁₋₂alkoxy;
provided than when R⁰ is hydrogen or methyl, a is 0, R³ is hydrogen, R⁴ is methoxy, and b is 0, then R² is other than pyrazol-4-yl;
or an isotopologue or pharmaceutically acceptable salt thereof.

4. The compound of claim 3, wherein
R⁰ is selected from the group consisting of hydrogen, methyl, trifluoromethyl and 3-amino-pyrrolidin-1-yl;
a is an integer from 0 to 1;
R¹ is selected from the group consisting of 5-hydroxy, 5-chloro, 5-fluoro, 5-methoxy, 5-cyano, 7-fluoro, 7-methoxy and 8-fluoro;
R² is selected from the group consisting of pyrazol-4-yl, 3-methyl-pyrazol-4-yl, 3-amino-pyrazol-4-yl, 3-trifluoromethyl-pyrazol-4-yl, pyrimidin-5-yl, 2-amino-pyrimidin-4-yl, pyridin-3-yl, pyridin-4-yl, 6-fluoro-pyridin-3-yl, 1,2,5-triazol-3-yl, 1,2,4-triazol-3-yl-4-one, 1,2,3-5-tetrazol-4-yl, pyridazin-5-yl-3-one and 1H-pyrrolo[2,3-b]pyridin-3-yl;
R³ is selected from the group consisting of hydrogen, methyl, S-methyl, R-methyl, hydroxymethyl, ethyl, 2-hydroxy-ethyl, —(CH₂CH₂)—NH(CH₃), —(CH₂CH₂)—N(CH₃)₂, —C(O)OH, —C(O)—OCH₃, —CH₂—SO₂—CH₃ and tetrahydro-thiopyran-4-yl 1,1-dioxide;
R⁴ is selected from the group consisting of hydrogen, chloro, fluoro, hydroxy, methoxy, ethoxy, difluoromethoxy, trifluoromethoxy, —O—CH₂—C(O)OH, —O—CD₂-C(O)OH, —O—CD₂-C(O)—NH(cyclopropyl), —O—CH₂CH₂—SO₂—CH₃, —O-(4-fluorophenyl), —O-benzyl, —O-(1-methyl-azetidin-3-yl), —C(O)—CH₃, —C(O)—NH-(4-fluorobenzyl), —C(O)—NH-(2,6-difluorobenzyl), —C(O)—NH-(1-methyl-azetidin-3-yl), —NH—SO₂—CH₃, —SO₂—NH₂, and oxazol-2-yl;
b is an integer from 0 to 2;
(R⁵)_{b} is selected from the group consisting of selected from the group consisting of 4-fluoro, 4-methoxy, 5-fluoro, 6-fluoro and 6-methoxy and 2,6-difluoro;
provided than when R⁰ is hydrogen or methyl, a is 0, R³ is hydrogen, R⁴ is methoxy, and b is 0, then R² is other than pyrazol-4-yl;
or a pharmaceutically acceptable salt thereof.

5. The compound of claim 4, wherein
R⁰ is selected from the group consisting of hydrogen, methyl, trifluoromethyl and 3-amino-pyrrolidin-1-yl;
a is an integer from 0 to 1;
R¹ is selected from the group consisting of 5-hydroxy, 5-chloro, 5-fluoro, 5-methoxy, 5-cyano, 7-fluoro, 7-methoxy and 8-fluoro;
R² is selected from the group consisting of pyrazol-4-yl, 3-methyl-pyrazol-4-yl, 2-amino-pyrazol-4-yl, 1,2,5-triazol-3-yl, 1,2,4-triazol-3-yl-4-one and 1H-pyrrolo[2,3-b]pyridin-3-yl;
R³ is selected from the group consisting of hydrogen, methyl, R-methyl, ethyl, —CH₂OH, —CH₂CH₂—NH(CH₃) and —CH₂CH₂—N(CH₃)₂;
R⁴ is selected from the group consisting of hydrogen, chloro, hydroxy, methoxy, ethoxy, difluoromethoxy, —O—CH₂—C(O)OH, —O—CD₂-C(O)OH, —O—CD₂-C(O)—NH(cyclopropyl), —O-(4-fluorophenyl), —O-benzyl, —C(O)—CH₃, —C(O)—NH-(4-fluorobenzyl), —C(O)—NH-(2,6-difluorobenzyl), —C(O)—NH-(1-methyl-azetidin-3-yl), —NH—SO₂—CH₃, —SO₂—NH₂ and oxazol-2-yl;
b is an integer from 0 to 2;
(R⁵)_{b} is selected from the group consisting of selected from the group consisting of 4-fluoro, 5-fluoro and 2,6-difluoro;
provided than when R⁰ is hydrogen or methyl, a is 0, R³ is hydrogen, R⁴ is methoxy, and b is 0, then R² is other than pyrazol-4-yl;
or a pharmaceutically acceptable salt thereof.

6. The compound of claim 3, wherein
R⁰ is selected from the group consisting of hydrogen, methyl and 3-amino-pyrrolidin-1-yl;
a is an integer from 0 to 1;
R¹ is selected from the group consisting of 5-hydroxy, 5-chloro, 5-fluoro, 5-methoxy, 5-cyano, 7-methoxy and 8-fluoro;
R² is selected from the group consisting of pyrazol-4-yl, 3-methyl-pyrazol-4-yl and 1,2,5-triazol-3-yl;
R³ is selected from the group consisting of hydrogen, methyl, R-methyl, —CH₂OH, —CH₂CH₂—NH(CH₃) and —CH₂CH₂—N(CH₃)₂;
R⁴ is selected from the group consisting of hydrogen, chloro, hydroxy, methoxy, ethoxy, —O—CH₂—C(O)OH, —O—CD₂-C(O)—NH(cyclopropyl), —O-(4-fluorophenyl), —O-benzyl, —C(O)—CH₃, —C(O)—NH-(4-fluorobenzyl), —C(O)—NH-(2,6-difluorobenzyl), —C(O)—NH-(1-methyl-azetidin-3-yl), —NH—SO₂—CH₃ and —SO₂—NH₂;
b is an integer from 0 to 2;
(R⁵)_{b} is selected from the group consisting of selected from the group consisting of 4-fluoro, 5-fluoro and 2,6-difluoro;
provided than when R⁰ is hydrogen or methyl, a is 0, is 8-methyl; R³ is hydrogen, R⁴ is methoxy, and b is 0, then R² is other than pyrazol-4-yl;
or a pharmaceutically acceptable salt thereof.

7. The compound of claim 3, wherein
R⁰ is selected from the group consisting of hydrogen, methyl and 3-amino-pyrrolidin-1-yl;
a is an integer from 0 to 1;
R¹ is selected from the group consisting of 5-hydroxy, 5-chloro, 5-fluoro, 5-methoxy, 5-cyano, and 7-methoxy;
R² is pyrazol-4-yl;
R³ is selected from the group consisting of hydrogen, methyl, R-methyl, —CH₂OH and —CH₂CH₂—NH(CH₃);
R⁴ is selected from the group consisting of chloro, hydroxy, methoxy, ethoxy, —O—CD₂-C(O)—NH(cyclopropyl), —C(O)—NH-(4-fluorobenzyl), —C(O)—NH-(2,6-difluorobenzyl), —NH—SO₂—CH₃ and —SO₂—NH₂;
b is an integer from 0 to 2;
(R⁵)_{b} is selected from the group consisting of selected from the group consisting of 4-fluoro, 5-fluoro and 2,6-difluoro;
provided than when R⁰ is hydrogen or methyl, a is 0, R³ is hydrogen, R⁴ is methoxy, and b is 0, then R² is other than pyrazol-4-yl;
or a pharmaceutically acceptable salt thereof.

8. The compound of claim 3, wherein
R⁰ is hydrogen;
a is an integer from 0 to 1;
R¹ is selected from the group consisting of 5-hydroxy, 5-fluoro, 5-methoxy and 5-cyano;
R² is pyrazol-4-yl;
R³ is selected from the group consisting of hydrogen, methyl and R-methyl;
R⁴ is selected from the group consisting of hydroxy, methoxy, —C(O)—NH-(4-fluorobenzyl) and —C(O)—NH-(2,6-difluorobenzyl);
b is an integer from 0 to 2;
(R⁵)_{b} is selected from the group consisting of selected from the group consisting of 5-fluoro and 2,6-difluoro;
provided than when R⁰ is hydrogen or methyl, a is 0, R³ is hydrogen, R⁴ is methoxy, b is 0, then R² is other than pyrazol-4-yl;
or a pharmaceutically acceptable salt thereof.

9. The compound of claim 3, wherein
3-[(3-methoxyphenyl)methyl]-4-oxo-6-(1H-pyrazol-4-yl)quinazoline-5-carbonitrile;
5-fluoro-3-[(3-methoxyphenyl)methyl]-6-(1H-pyrazol-4-yl)quinazolin-4-one;
3-[(1R)-1-(3-methoxyphenyl)ethyl]-6-(1H-pyrazol-4-yl)quinazolin-4-one;
5-methoxy-3-[(3-methoxyphenyl)methyl]-6-(1H-pyrazol-4-yl)quinazolin-4-one;
3-[3-(dimethylamino)-1-(3-methoxyphenyl)propyl]-6-(1H-pyrazol-4-yl)quinazolin-4-one;
N-(4-fluorophenyl)-3-[[4-oxo-6-(1H-pyrazol-4-yl)quinazolin-3-yl]methyl]benzamide;
N-[(2,6-difluorophenyl)methyl]-3-[[4-oxo-6-(1H-pyrazol-4-yl)quinazolin-3-yl]methyl]benzamide;
N-[(4-fluorophenyl)methyl]-3-[[4-oxo-6-(1H-pyrazol-4-yl)quinazolin-3-yl]methyl]benzamide;
or a pharmaceutically acceptable salt thereof.

10. A compound selected from the group consisting of
N-(4-fluorobenzyl)-3-(4-oxo-6-(1H-pyrazol-4-yl)quinazolin-3(4H)-yl)benzamide;
N-(2,4-difluorobenzyl)-4-((4-oxo-6-(1H-pyrrol-3-yl)quinazolin-3(4H)-yl)methyl)benzamide;
N-(2,6-difluorobenzyl)-4-((4-oxo-6-(1H-pyrrol-3-yl)quinazolin-3(4H)-yl)methyl)benzamide;
3-(benzo[d][1,3]dioxol-4-ylmethyl)-6-(1H-pyrazol-4-yl)quinazolin-4(3H)-one;
N-(4-fluorobenzyl)-4-((4-oxo-6-(1H-pyrazol-4-yl)quinazolin-3(4H)-yl)methyl)picolinamide;
3-(3-methoxybenzyl)-6-(2-((2-methoxyethyl)amino)pyrimidin-4-yl)quinazolin-4(3H)-one;
and isomers and pharmaceutically acceptable salts thereof.

11. A pharmaceutical composition comprising a pharmaceutically acceptable carrier and the compound of claim 1.

12-13. (canceled)

14. A method of treating a disorder mediated by GRK2 activity, comprising administering to a subject in need thereof a therapeutically effective amount of the compound of claim 1.

15. The method of claim 14, wherein the disorder mediated by GRK2 activity is selected from the group consisting of obesity, excess weight, impaired glucose tolerance (IGT), impaired fasting glucose (IFT), gestational diabetes, Type II diabetes mellitus, Syndrome X (also known as Metabolic Syndrome), nephropathy, neuropathy, retinopathy, cardiac failure, cardiac hypertrophy, cardiac fibrosis, hypertension, angina, atherosclerosis, heart disease, heart attack, ischemia, stroke, nerve damage or poor blood flow in the feet, sepsis-associated encephalopathy (SAE), non-alcoholic steatohepatitis (NASH), non-alcoholic fatty liver disease (NAFLD), end-stage kidney disease, chronic kidney disease, acute renal failure, nephrotic syndrome, renal hyperfiltrative injury, hyperfiltrative diabetic nephropathy, renal hyperfiltration, glomerular hyperfiltration, renal allograft hyperfiltration, compensatory hyperfiltration, hyperfiltrative chronic kidney disease, hyperfiltrative acute renal failure and a measured GFR equal or greater than 125 mL/min/1.73 m².

16. The method of claim 14, wherein the disorder mediated by GRK2 activity is selected from the group consisting of obesity, excess weight, impaired glucose tolerance (IGT), impaired fasting glucose (IFT), gestational diabetes, Type II diabetes mellitus, Syndrome X (also known as Metabolic Syndrome), diabetic nephropathy, diabetic neuropathy, diabetic retinopathy, cardiac failure, cardiac hypertrophy, hypertension, angina, atherosclerosis, non-alcoholic steatohepatitis (NASH), non-alcoholic fatty liver disease (NAFLD), end-stage kidney disease, chronic kidney disease, acute renal failure, and a measured GFR equal or greater than 125 mL/min/1.73 m²

17. The method of claim 14, wherein the disorder mediated by GRK2 activity is selected from the group consisting of obesity, excess weight, impaired glucose tolerance (IGT), impaired fasting glucose (IFT), gestational diabetes, Type II diabetes mellitus, Syndrome X (also known as Metabolic Syndrome), diabetic nephropathy, diabetic neuropathy, diabetic retinopathy, non-alcoholic steatohepatitis (NASH), non-alcoholic fatty liver disease (NAFLD), end-stage kidney disease, chronic kidney disease, acute renal failure, and a measured GFR equal or greater than 125 mL/min/1.73 m².

18-24. (canceled)

25. A compound of formula (D) 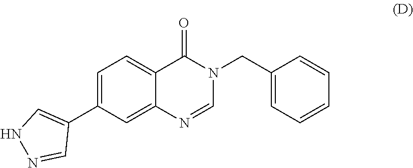 [SMILES: O=c1c2ccc(-c3cn[nH]c3)cc2ncn1Cc1ccccc1]
or isomer or pharmaceutically acceptable salt thereof.

26. (canceled)
